(19) United States
(12) Patent Application Publication
(10) Pub. No.: US 2014/0330298 A1
(43) Pub. Date: Nov. 6, 2014

(54) CLAMP ARM FEATURES FOR ULTRASONIC SURGICAL INSTRUMENT

(71) Applicant: Ethicon Endo-Surgery, Inc., Cincinnati, OH (US)

(72) Inventors: Susan G. Arshonsky, Cincinnati, OH (US); Jonathan T. Batross, Cincinnati, OH (US); William D. Dannaher, Cincinnati, OH (US); Craig T. Davis, Cincinnati, OH (US); Kristen G. Denzinger, Cincinnati, OH (US); Carl J. Draginoff, Jr., Mason, OH (US); Craig N. Faller, Milford, OH (US); Kevin D. Felder, Cincinnati, OH (US); Thomas C. Gallmeyer, Cincinnati, OH (US); Jacob S. Gee, Cincinnati, OH (US); Kevin L. Houser, Springboro, OH (US); Michael R. Lamping, Cincinnati, OH (US); Amy L. Marcotte, Mason, OH (US); Jeffrey D. Messerly, Cincinnati, OH (US); Rudolph H. Nobis, Mason, OH (US); John B. Schulte, West Chester, OH (US); Richard C. Smith, Milford, OH (US); Richard W. Timm, Cincinnati, OH (US)

(21) Appl. No.: 14/257,245

(22) Filed: Apr. 21, 2014

Related U.S. Application Data
(60) Provisional application No. 61/819,050, filed on May 3, 2013.
(60) Provisional application No. 61/879,700, filed on Sep. 19, 2013.

(51) Int. Cl. A61B 17/32

(52) U.S. Cl. CPC A61B 17/320092; A61B 2017/00115
USPC 606/169

(57) ABSTRACT

A surgical apparatus comprises a body, an ultrasonic transducer, a shaft, an acoustic waveguide, an articulation section, and an end effector. The ultrasonic transducer is operable to convert electrical power into ultrasonic vibrations. The shaft couples the end effector and the body together. The acoustic waveguide is coupled with the transducer. The end effector comprises a clamp arm and an ultrasonic blade in acoustic communication with the ultrasonic transducer. The clamp arm is configured to pivot toward and away from the ultrasonic blade. The shaft assembly comprises an inner tube configured to longitudinally translate to thereby pivot the clamp arm toward and away from the ultrasonic blade. A distal portion of the inner tube is operable to flex to thereby accommodate pivoting of the clamp arm.

### PRIORITY

This application claims priority to U.S. Provisional Patent App. No. 61/819,050, entitled “Clamp Arm,” filed May 3, 2013, the disclosure of which is incorporated by reference herein.

This application also claims priority to U.S. Provisional Patent App. No. 61/879,700, entitled “Ultrasonic Surgical Instrument,” filed Sep. 19, 2013, the disclosure of which is incorporated by reference herein.

### BACKGROUND

A variety of surgical instruments include an end effector having a blade element that vibrates at ultrasonic frequencies to cut and/or seal tissue (e.g., by denaturing proteins in tissue cells). These instruments include piezoelectric elements that convert electrical power into ultrasonic vibrations, which are communicated along an acoustic waveguide to the blade element. The precision of cutting and coagulation may be controlled by the surgeon's technique and adjusting the power level, blade edge, tissue traction and blade pressure.

Examples of ultrasonic surgical instruments include the HARMONIC ACE®

Ultrasonic Shears, the HARMONIC WAVE® Ultrasonic Shears, the HARMONIC FOCUS® Ultrasonic Shears, and the HARMONIC SYNERGY® Ultrasonic Blades, all by Ethicon Endo-Surgery, Inc. of Cincinnati, Ohio. Further examples of such devices and related concepts are disclosed in U.S. Pat. No. 5,322,055, entitled “Clamp Coagulator/Cutting System for Ultrasonic Surgical Instruments,” issued Jun. 21, 1994, the disclosure of which is incorporated by reference herein; U.S. Pat. No. 5,873,873, entitled “Ultrasonic Clamp Coagulator Apparatus Having Improved Clamp Mechanism,” issued Feb. 23, 1999, the disclosure of which is incorporated by reference herein; U.S. Pat. No. 5,980,510, entitled “Ultrasonic Clamp Coagulator Apparatus Having Improved Clamp Arm Pivot Mount,” filed Oct. 10, 1997, the disclosure of which is incorporated by reference herein; U.S. Pat. No. 6,325,811, entitled “Blades with Functional Balance Asymmetries for use with Ultrasonic Surgical Instruments,” issued Dec. 4, 2001, the disclosure of which is incorporated by reference herein; U.S. Pat. No. 6,773,444, entitled “Blades with Functional Balance Asymmetries for Use with Ultrasonic Surgical Instruments,” issued Aug. 10, 2004, the disclosure of which is incorporated by reference herein; and U.S. Pat. No. 6,783,524, entitled “Robotic Surgical Tool with Ultrasound Cauterizing and Cutting Instrument,” issued Aug. 31, 2004, the disclosure of which is incorporated by reference herein.

Still further examples of ultrasonic surgical instruments are disclosed in U.S. Pub.

No. 2006/0079874, entitled “Tissue Pad for Use with an Ultrasonic Surgical Instrument,” published Apr. 13, 2006, the disclosure of which is incorporated by reference herein; U.S. Pub. No. 2007/0191713, entitled “Ultrasonic Device for Cutting and Coagulating,” published Aug. 16, 2007, the disclosure of which is incorporated by reference herein; U.S. Pub. No. 2007/0282333, entitled “Ultrasonic Waveguide and Blade,” published Dec. 6, 2007, the disclosure of which is incorporated by reference herein; U.S. Pub. No. 2008/0200940, entitled “Ultrasonic Device for Cutting and Coagulating,” published Aug. 21, 2008, the disclosure of which is incorporated by reference herein; U.S. Pub. No. 2009/0105750, entitled “Ergonomic Surgical Instruments,” published Apr. 23, 2009, the disclosure of which is incorporated by reference herein; U.S. Pub. No. 2010/0069940, entitled “Ultrasonic Device for Fingertip Control,” published Mar. 18, 2010, the disclosure of which is incorporated by reference herein; and U.S. Pub. No. 2011/0015660, entitled “Rotating Transducer Mount for Ultrasonic Surgical Instruments,” published Jan. 20, 2011, the disclosure of which is incorporated by reference herein; and U.S. Pub. No. 2012/0029546, entitled “Ultrasonic Surgical Instrument Blades,” published Feb. 2, 2012, the disclosure of which is incorporated by reference herein.

Some of ultrasonic surgical instruments may include a cordless transducer such as that disclosed in U.S. Pub. No. 2012/0112687, entitled “Recharge System for Medical Devices,” published May 10, 2012, the disclosure of which is incorporated by reference herein; U.S. Pub. No. 2012/0116265, entitled “Surgical Instrument with Charging Devices,” published May 10, 2012, the disclosure of which is incorporated by reference herein; and/or U.S. Pat. App. No. 61/410,603, filed Nov. 5, 2010, entitled “Energy-Based Surgical Instruments,” the disclosure of which is incorporated by reference herein.

Additionally, some ultrasonic surgical instruments may include an articulating shaft section. Examples of such ultrasonic surgical instruments are disclosed in U.S. patent application Ser. No. 13/538,588, filed .29, 2012, entitled “Surgical Instruments with Articulating Shafts,” the disclosure of which is incorporated by reference herein; U.S. patent application Ser. No. 13/657,553, filed Oct. 22, 2012, entitled “Flexible Harmonic Waveguides/Blades for Surgical Instruments,” the disclosure of which is incorporated by reference herein; and U.S. patent application Ser. No. 14/028,717, filed Sep. 17, 2013, entitled “Articulation Features for Ultrasonic Surgical Instrument,” the disclosure of which is incorporated by reference herein.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a side elevational view of an exemplary surgical instrument;

FIG. 2 depicts a perspective view of an end effector and a shaft assembly of the instrument of FIG. 1;

FIG. 3 depicts a side elevational view of a clamp arm of the end effector of FIG. 2;

FIG. 4 depicts a perspective view of the clamp arm of FIG. 3;

FIG. 5 depicts a perspective view of the distal end of an inner tube of the shaft assembly of FIG. 2;

FIG. 6 depicts a side elevational view of the distal end of the inner tube of FIG. 5;

FIG. 7 depicts a top view of the distal end of the inner tube of FIG. 5;

FIG. 8 depicts a bottom view of the distal end of the inner tube of FIG. 5;

FIG. 9 depicts a perspective view of the distal end of an outer sheath of the shaft assembly of FIG. 2;

FIG. 10 depicts a side elevational view of the distal end of the outer sheath of FIG. 9;

FIG. 11 depicts a bottom view of the distal end of the outer sheath of FIG. 9;

FIG. 12A depicts a side elevational view of the end effector and shaft assembly of FIG. 2 with the clamp arm of FIG. 3 in a first rotational position and with the inner tube of FIG. 5 in a first longitudinal position;

FIG. 12B depicts a side elevational view of the end effector and shaft assembly of FIG. 2 with the clamp arm of FIG. 3 moved into a second rotational position by movement of the inner tube of FIG. 5 into a second longitudinal position, with the inner tube driven into a first flexed position;

FIG. 12C depicts a side elevational view of the end effector and shaft assembly of FIG. 2 with the clamp arm of FIG. 3 moved into a third rotational position by movement of the inner tube of FIG. 5 into a third longitudinal position, with the inner tube driven into a second flexed position;

FIG. 13 depicts a perspective view of the end effector and shaft assembly of FIG. 2, with the clamp arm of FIG. 3 in the first rotational position of FIG. 12A;

FIG. 14 depicts a perspective view of the distal end of an exemplary alternative outer sheath configured for use with the instrument of FIG. 1;

FIG. 15A depicts a side elevational view of the end effector and shaft assembly of FIG. 2 and the outer sheath of FIG. 14, with the clamp arm of FIG. 3 in a first rotational position and with an exemplary alternative inner tube in a first longitudinal position;

FIG. 15B depicts a side elevational view of the end effector and shaft assembly of

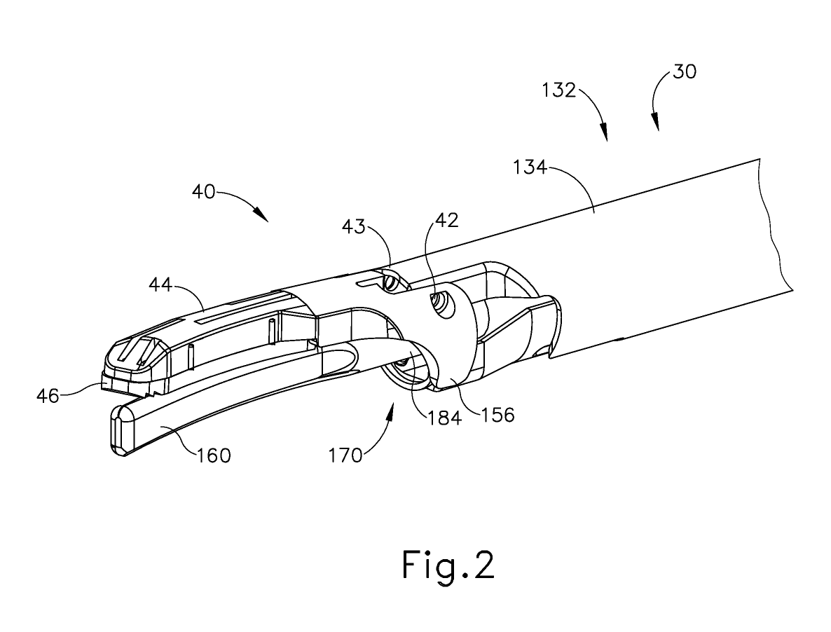

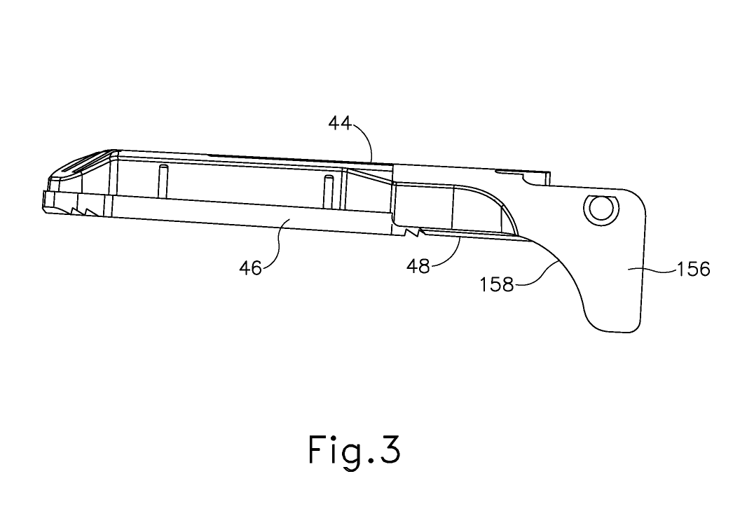

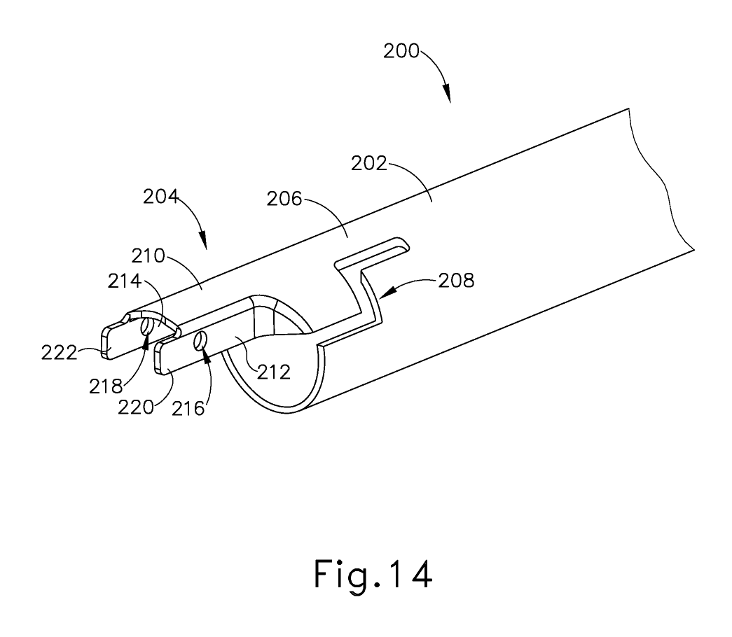

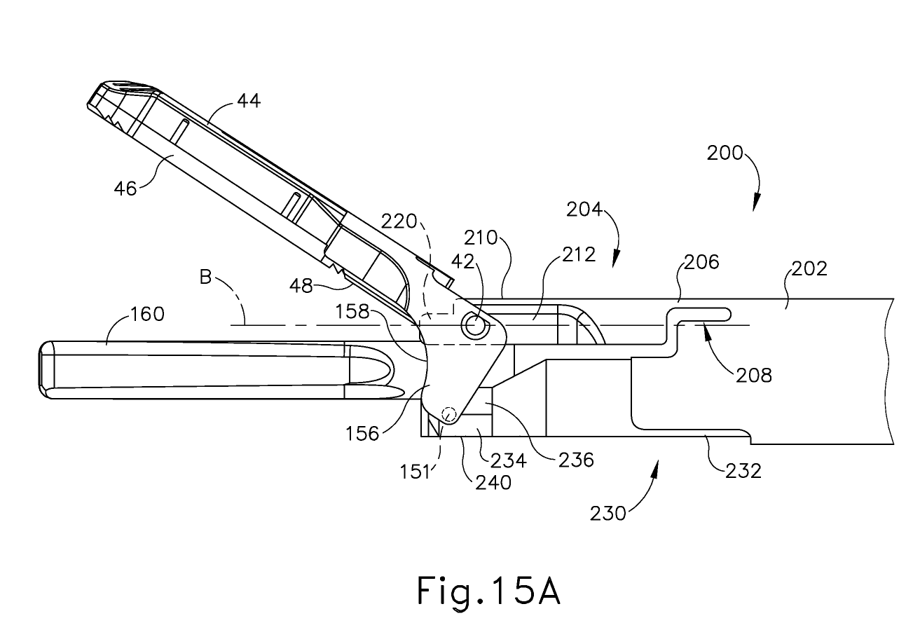

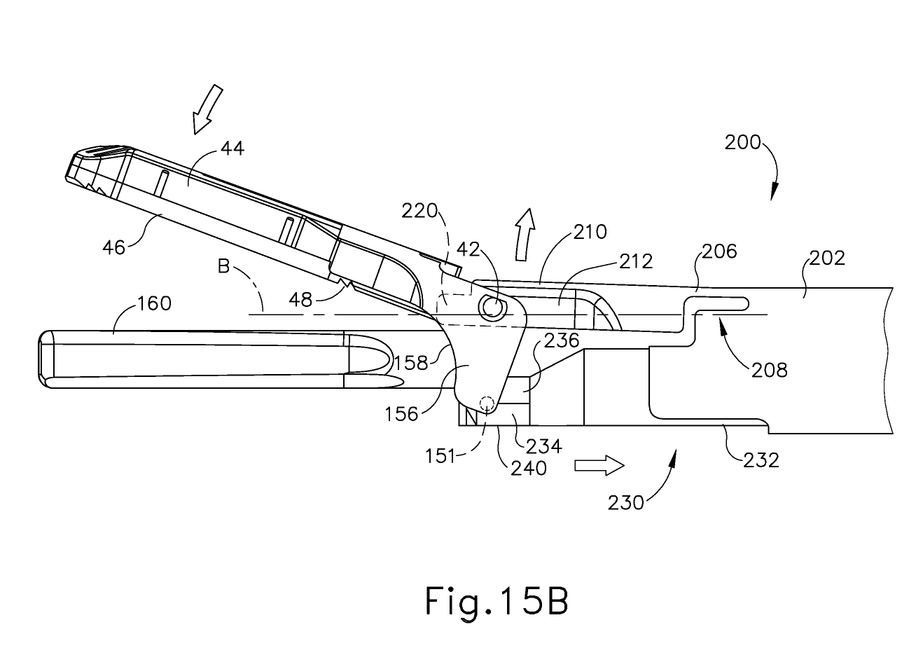

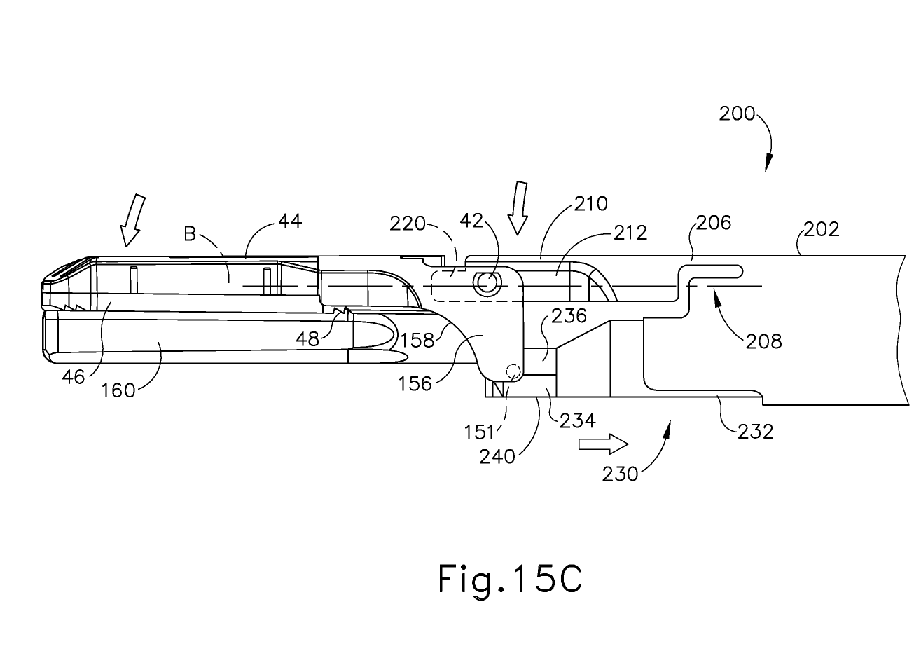

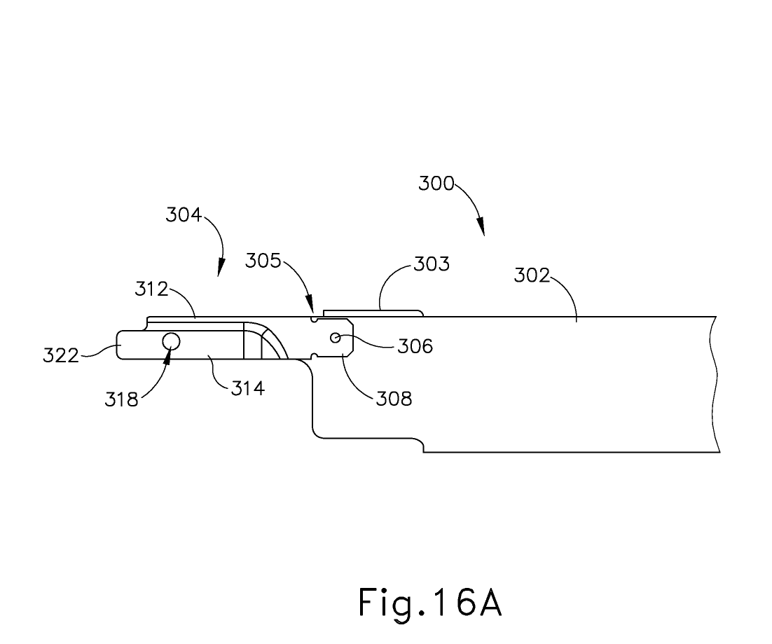

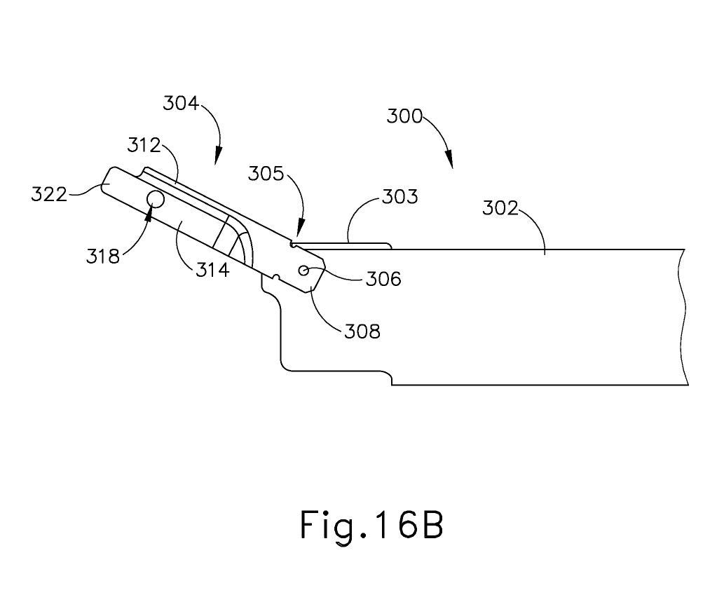

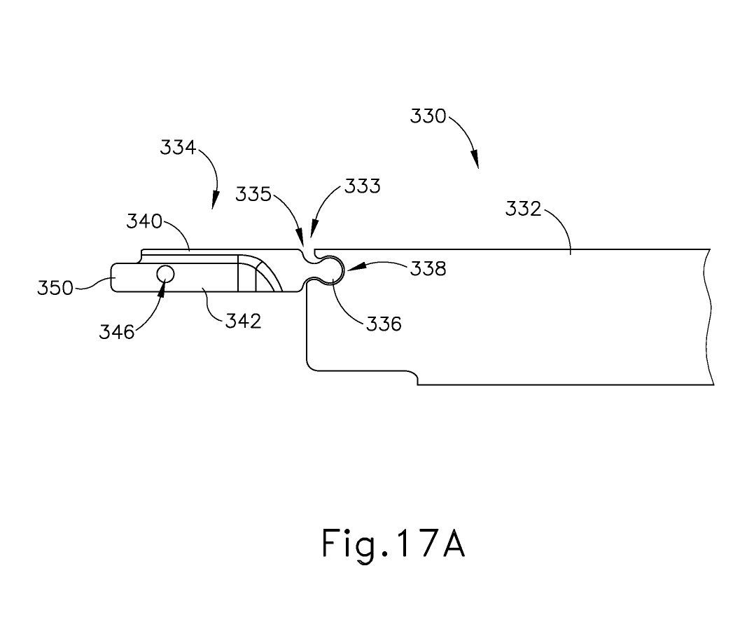

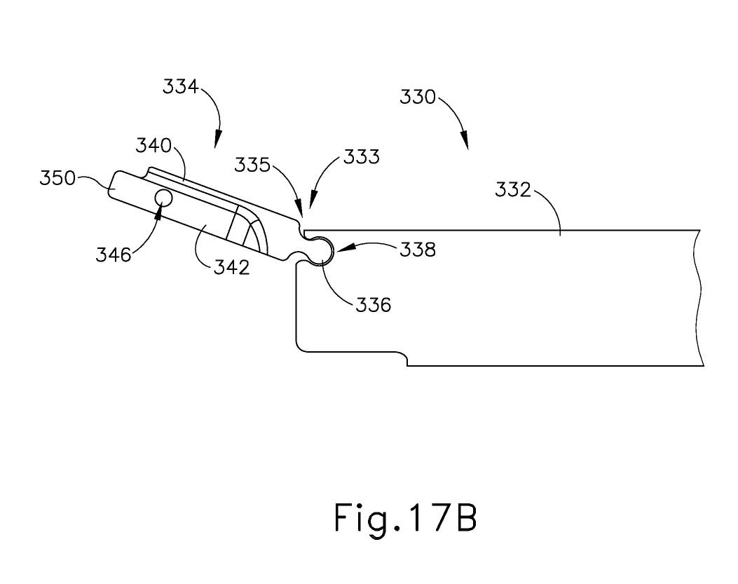

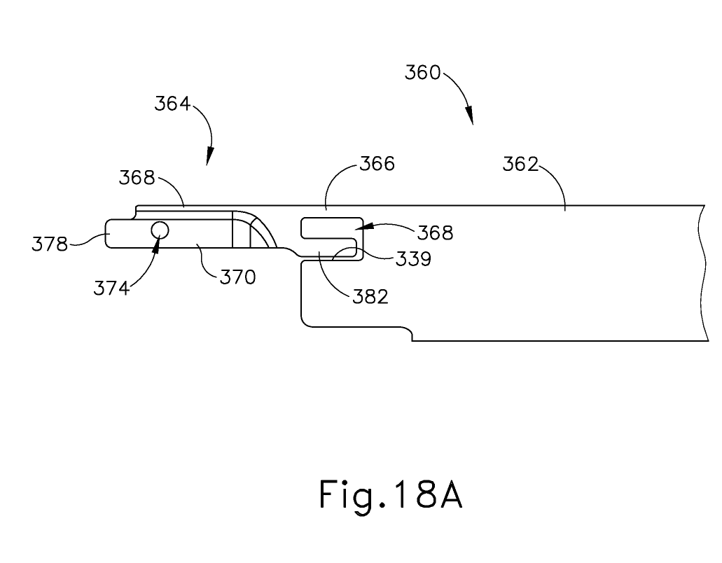

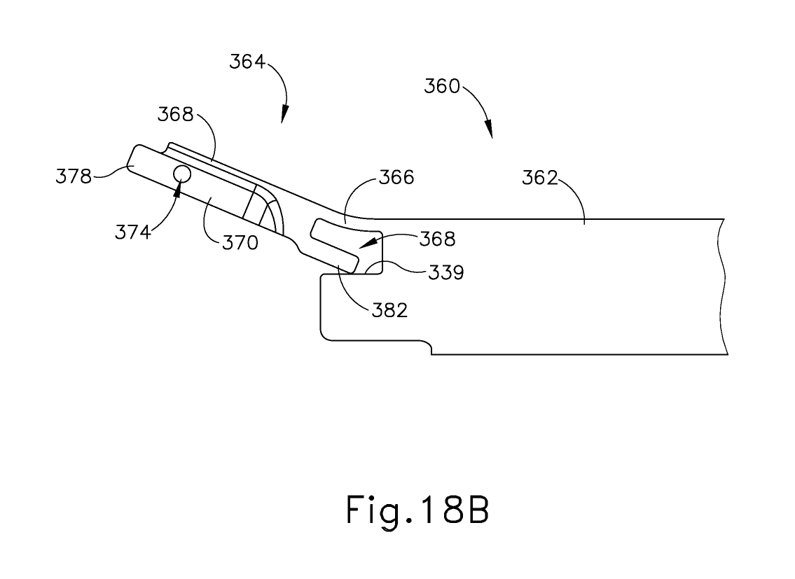

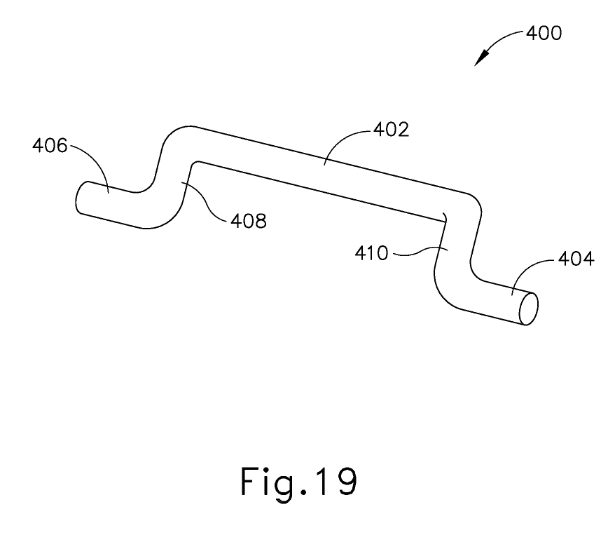

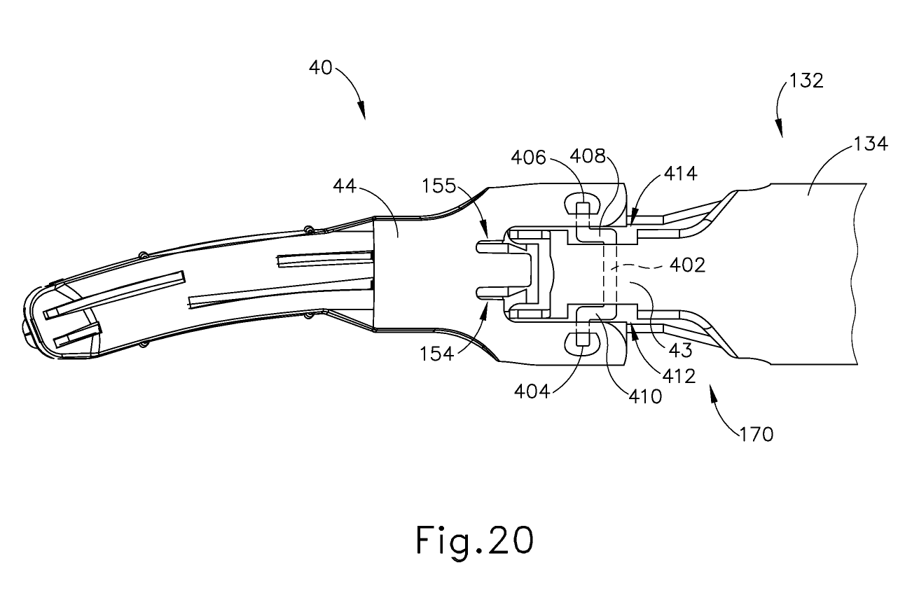

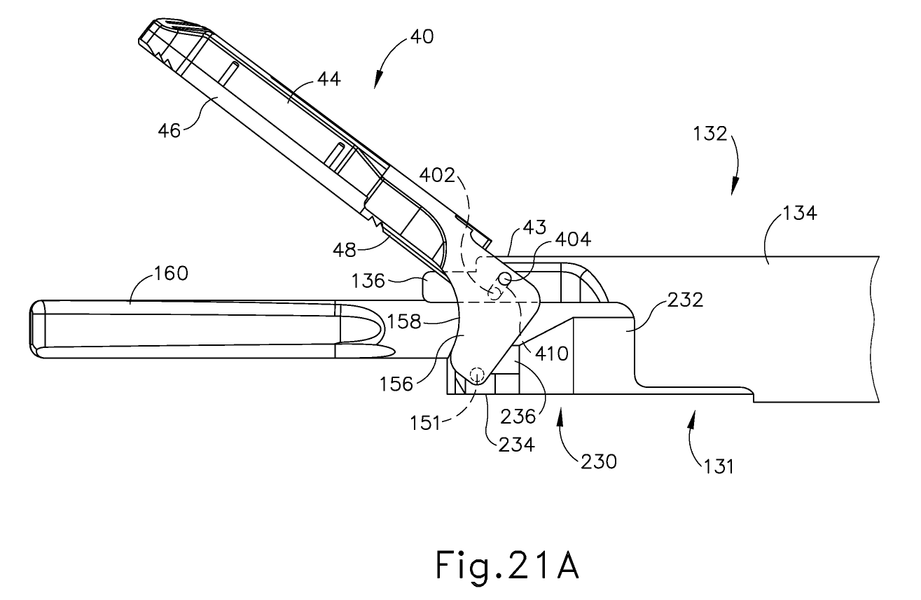

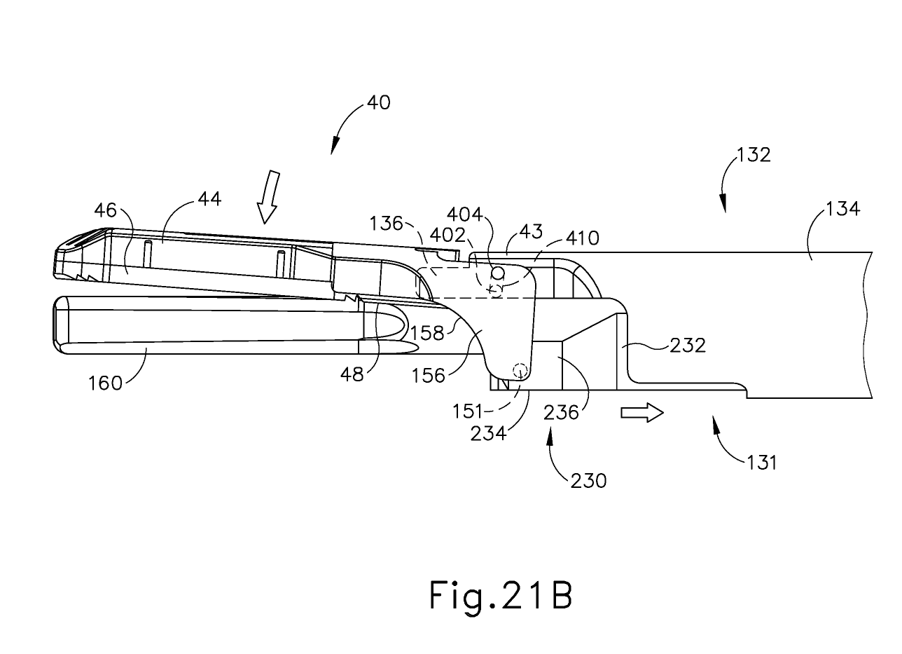

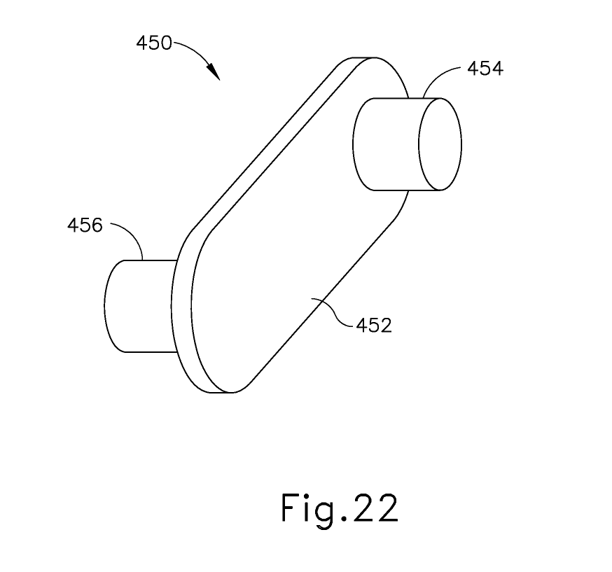

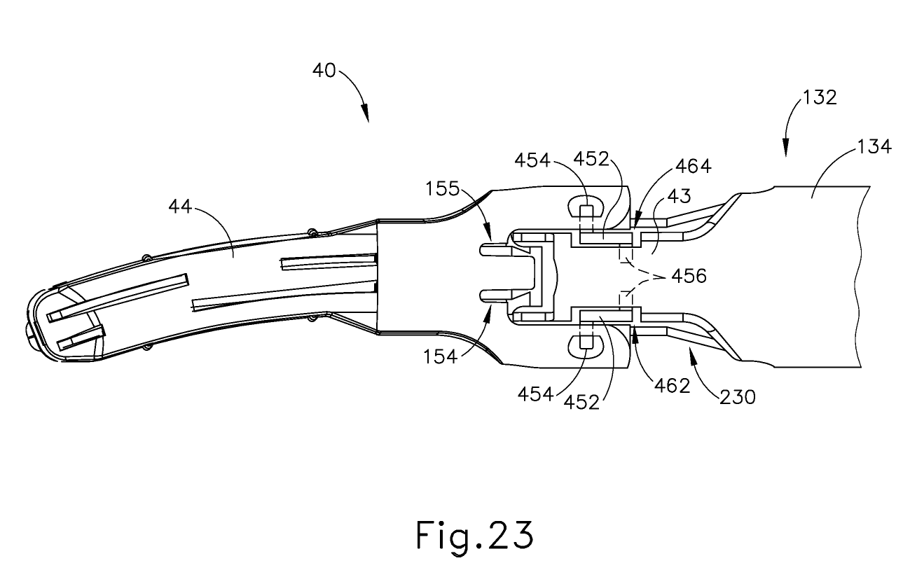

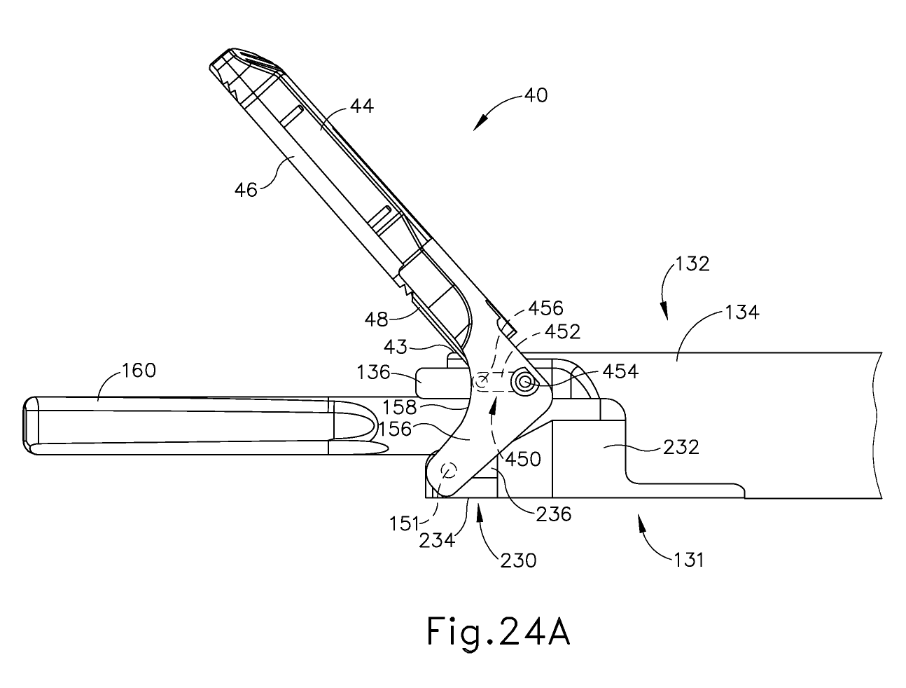

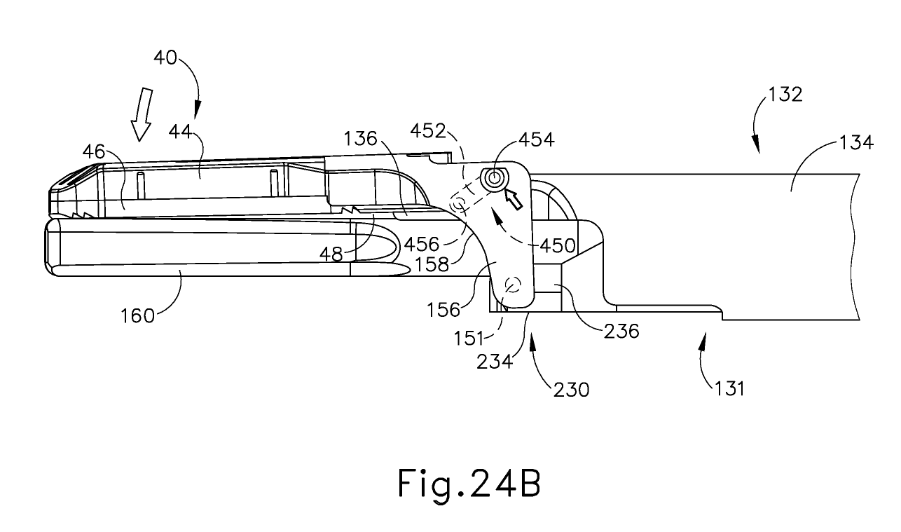

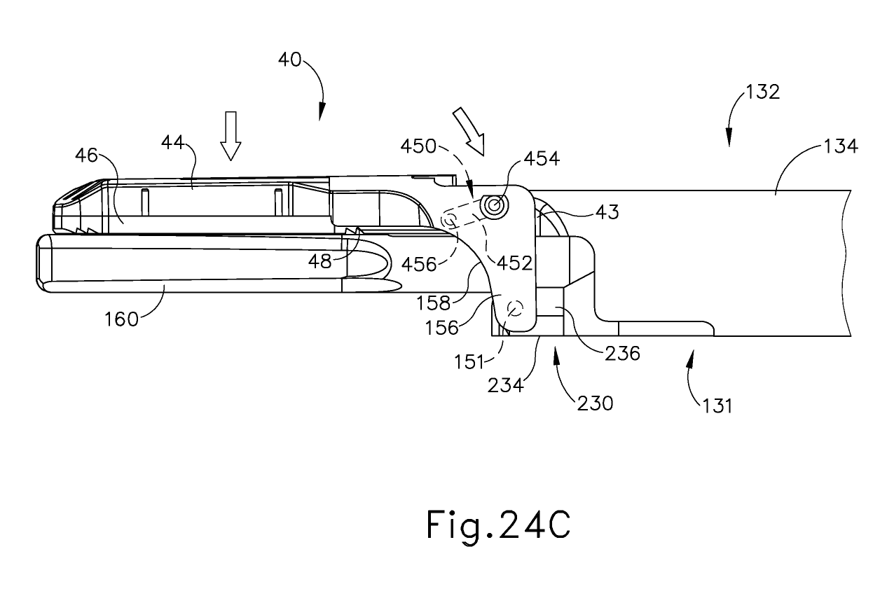

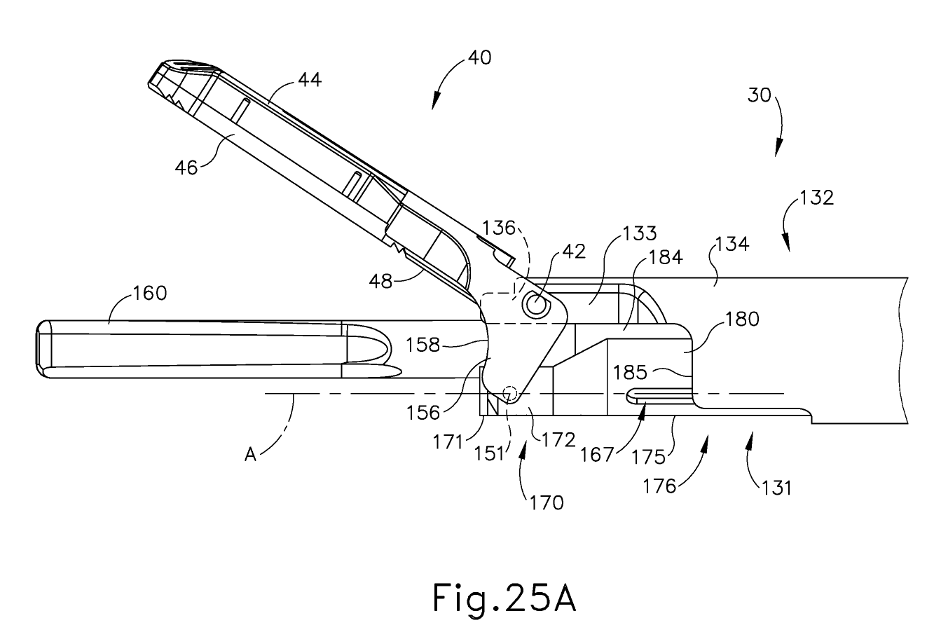

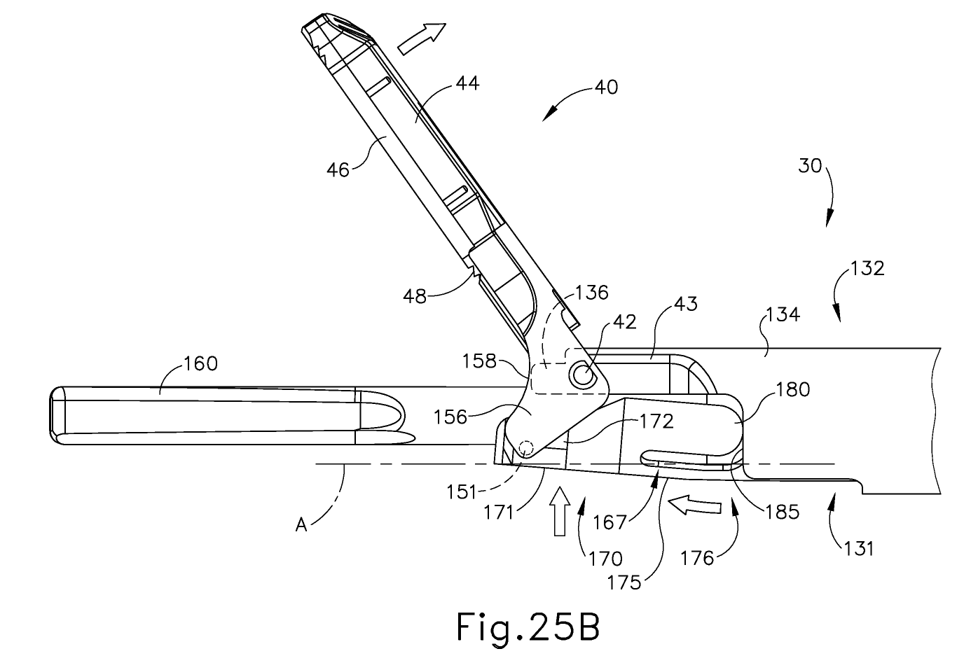

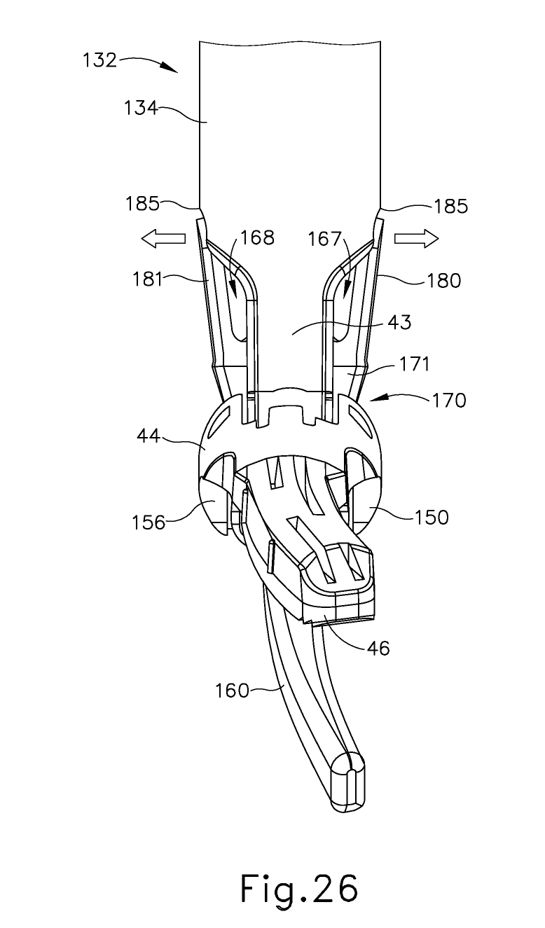

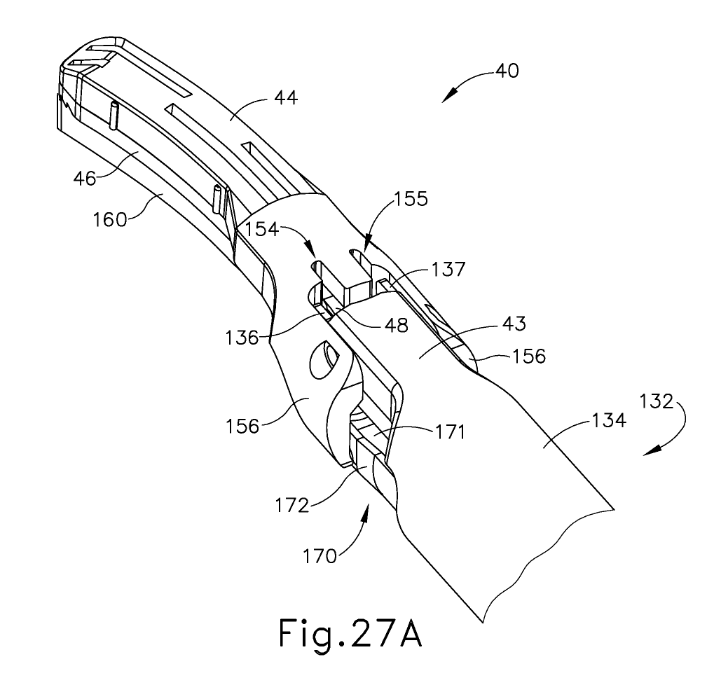

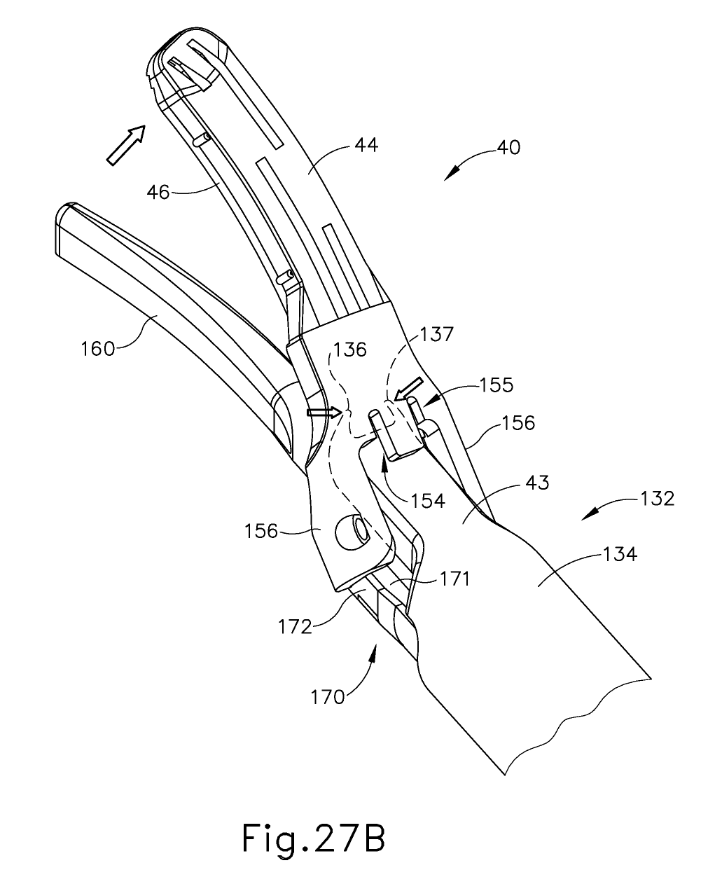

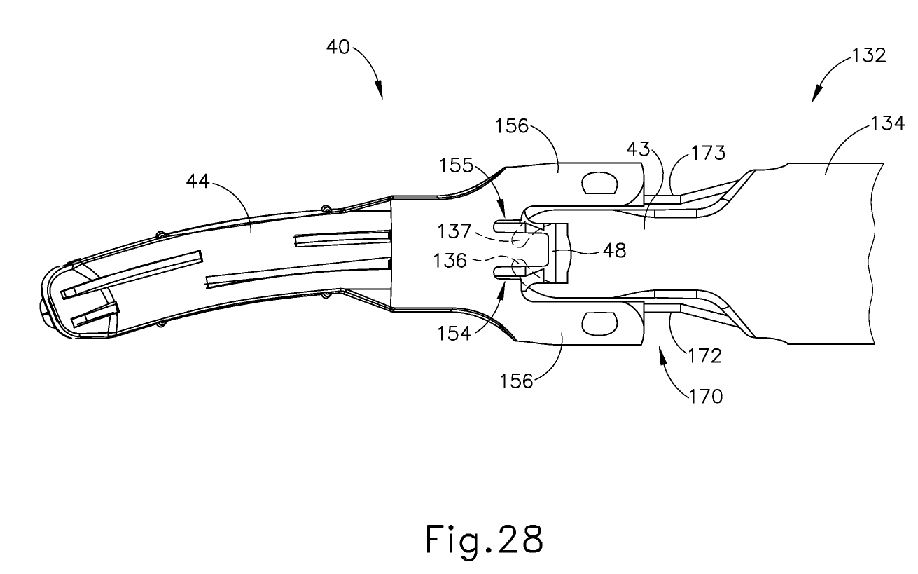

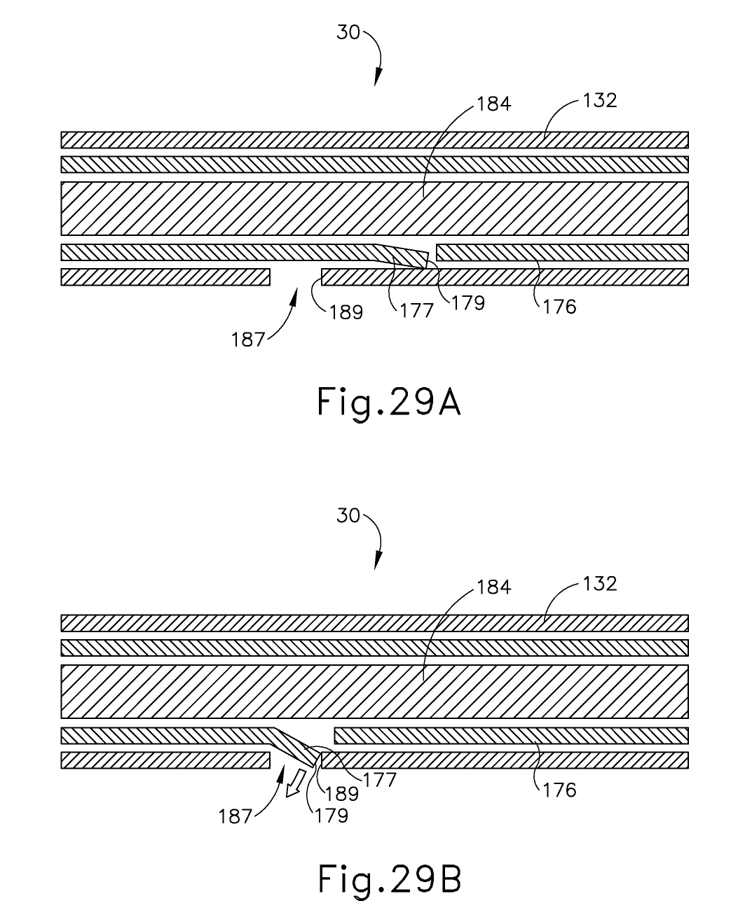

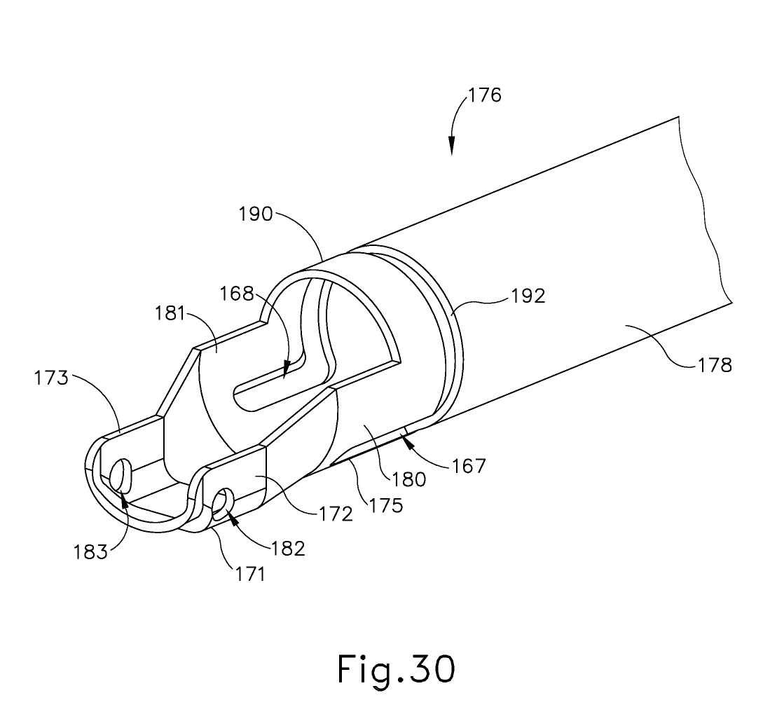

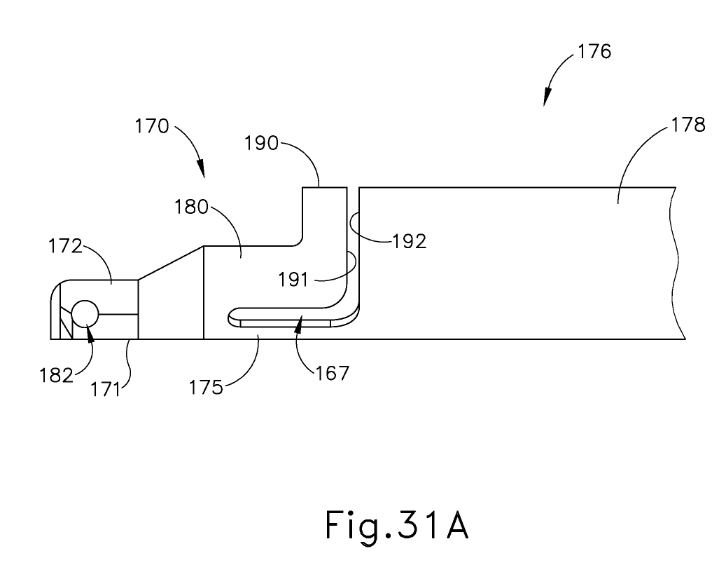

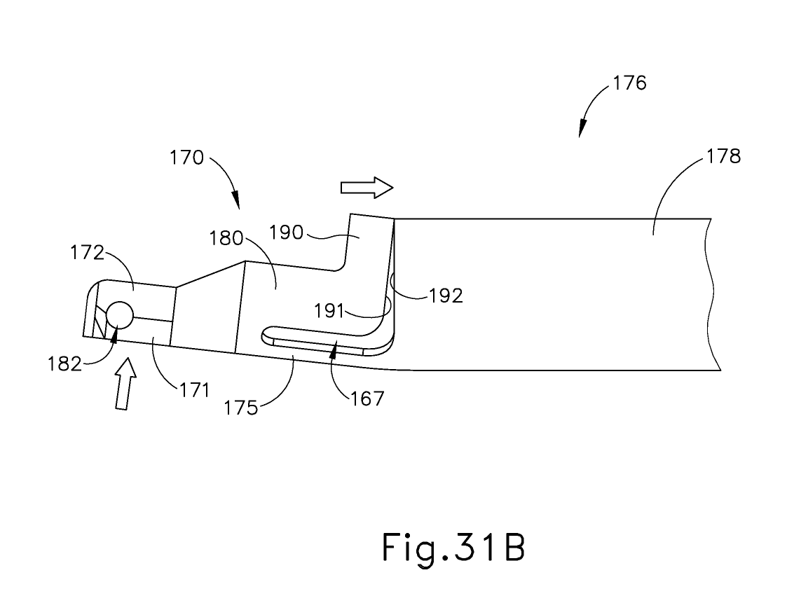

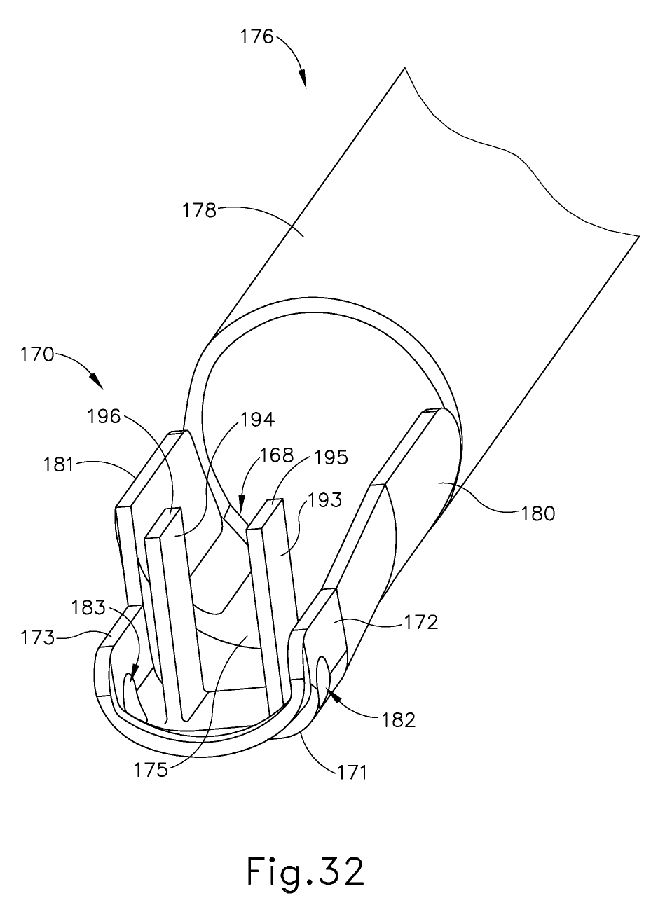

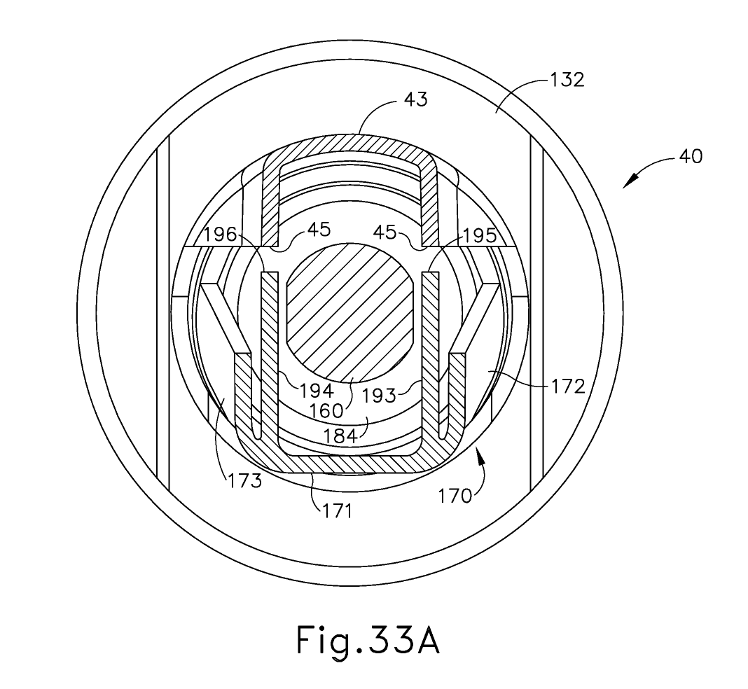

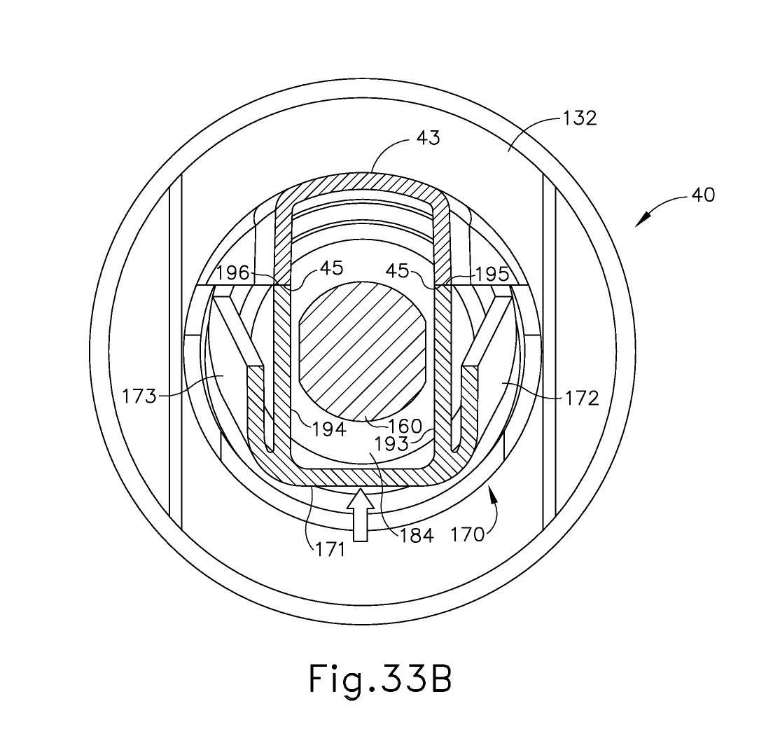

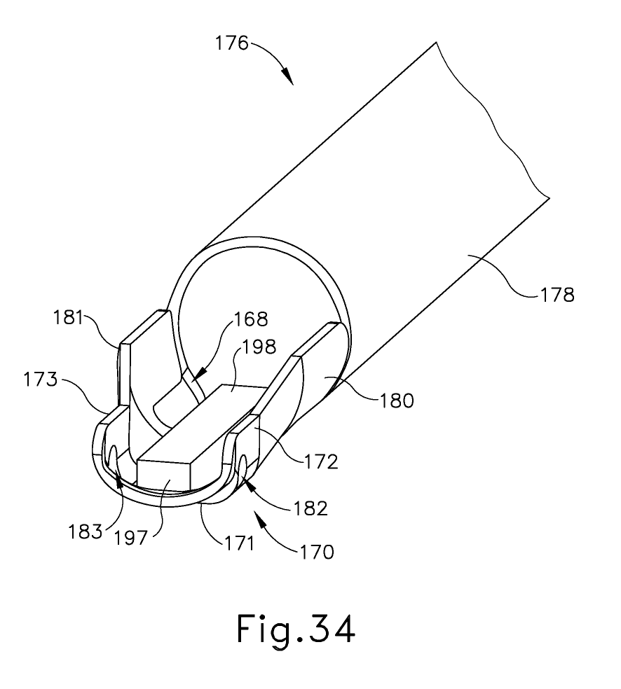

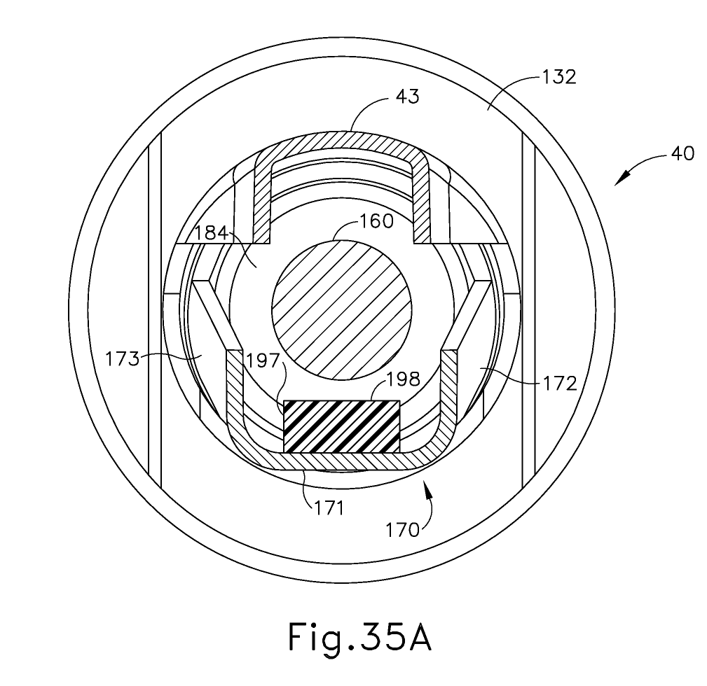

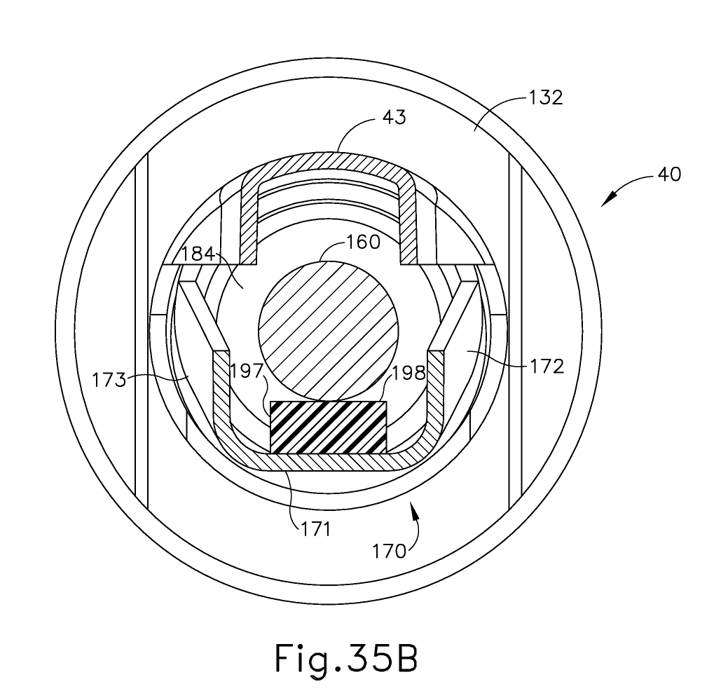

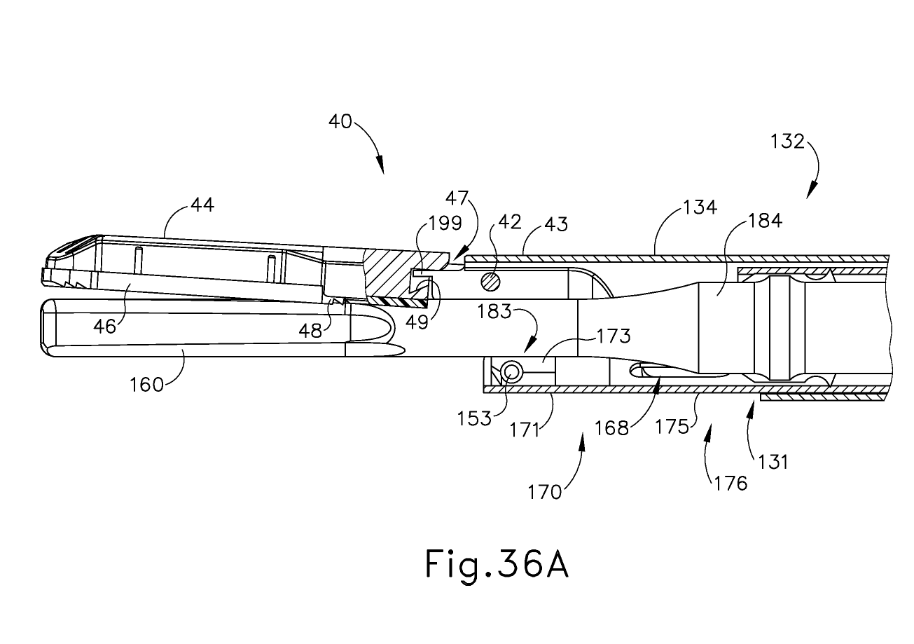

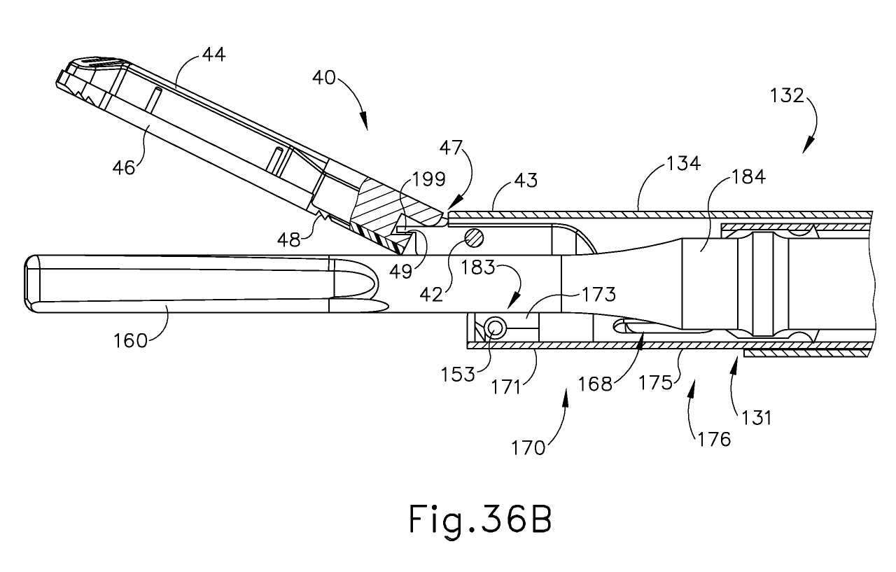

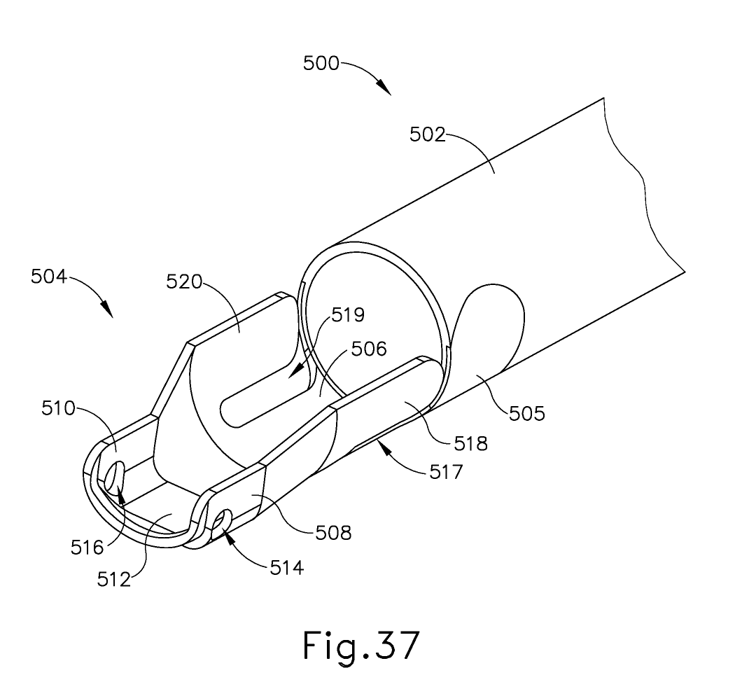

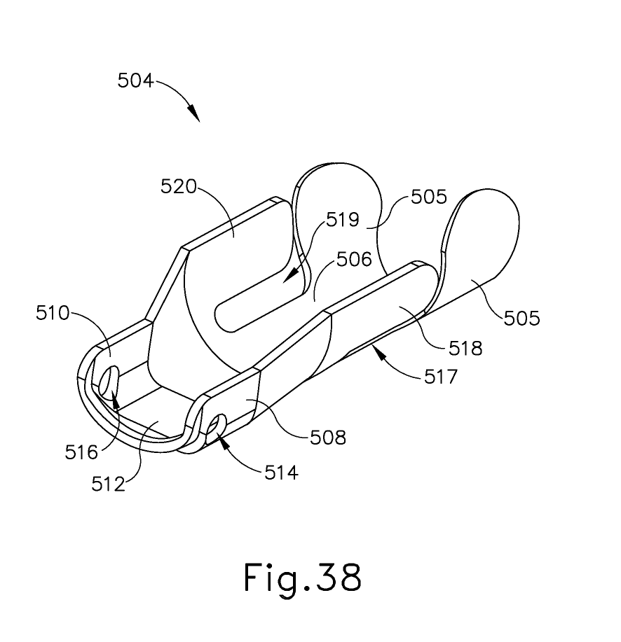

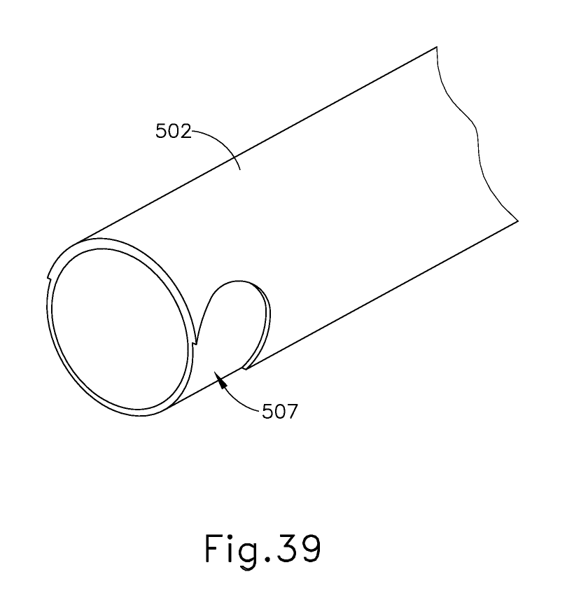

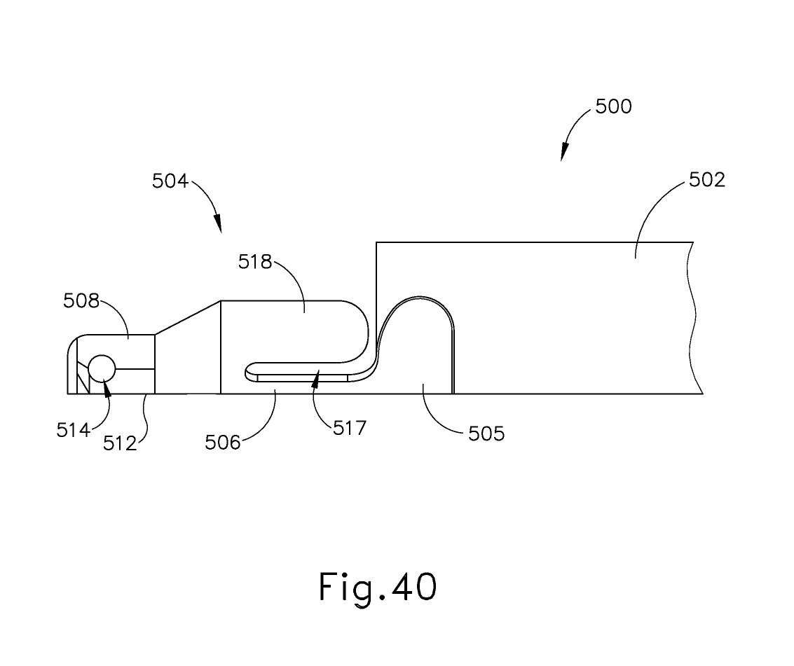

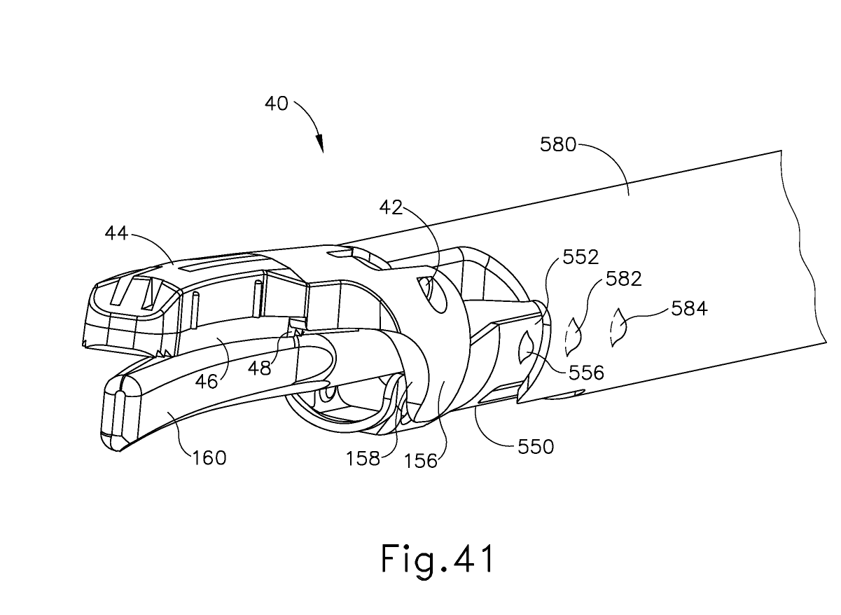

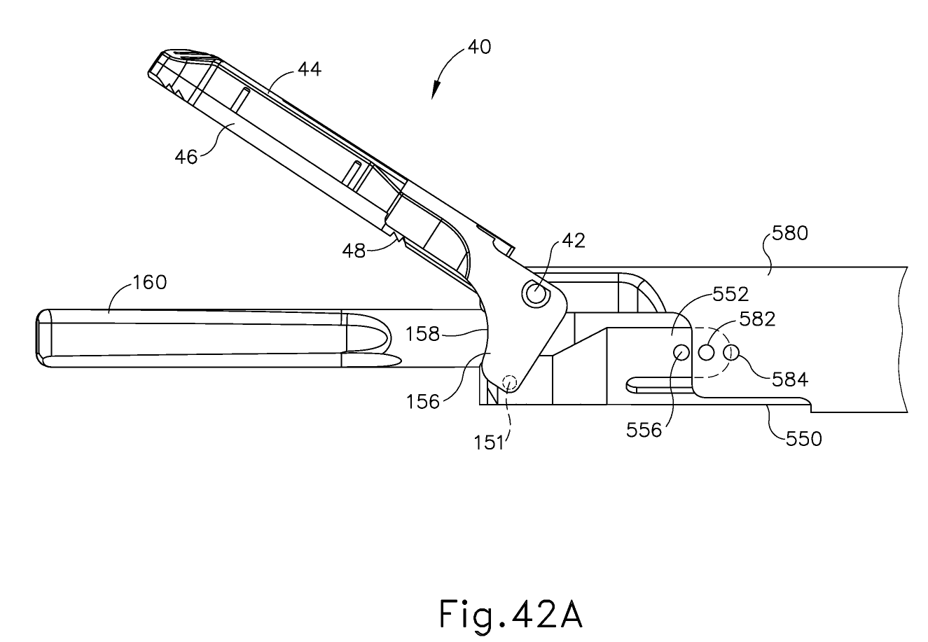

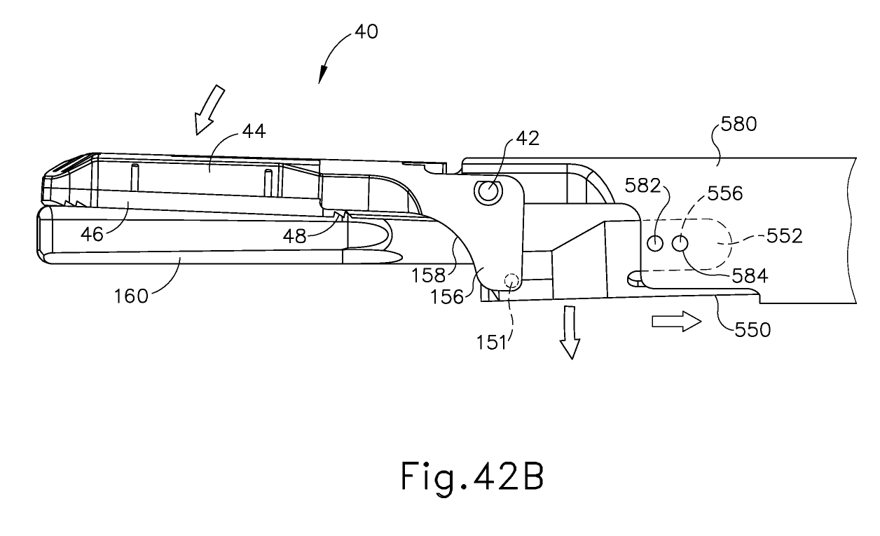

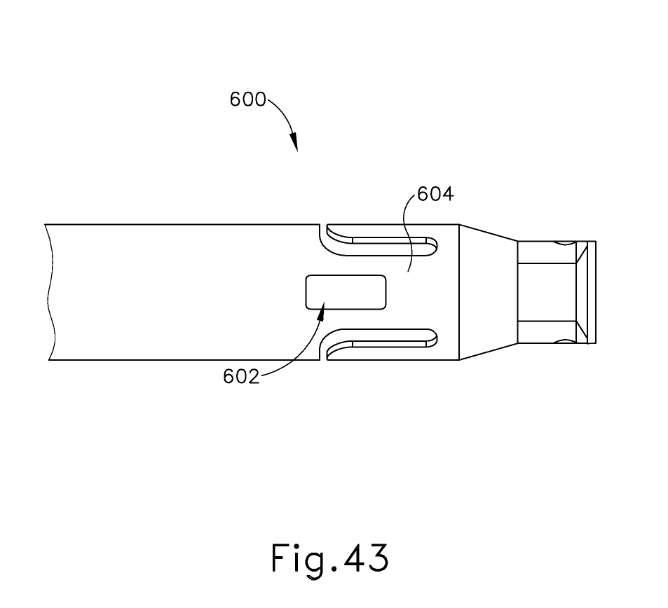

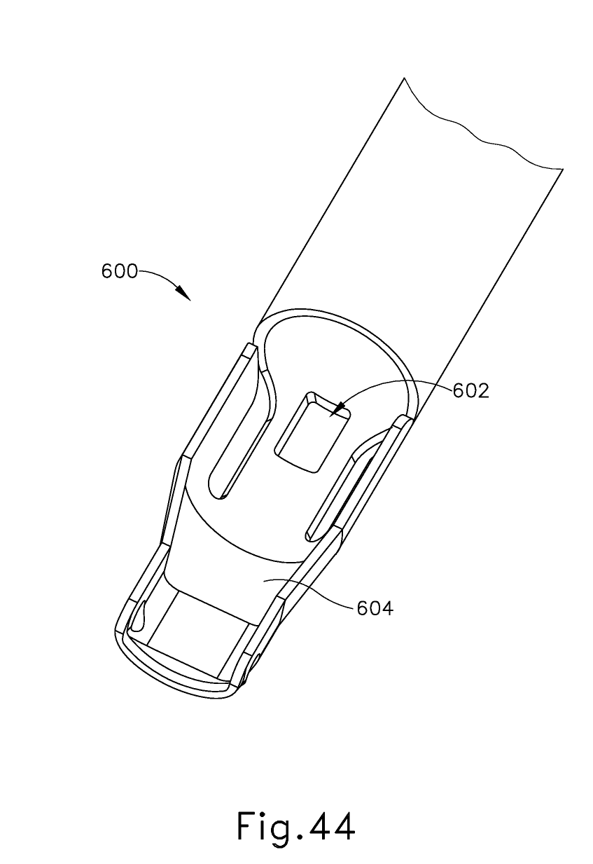

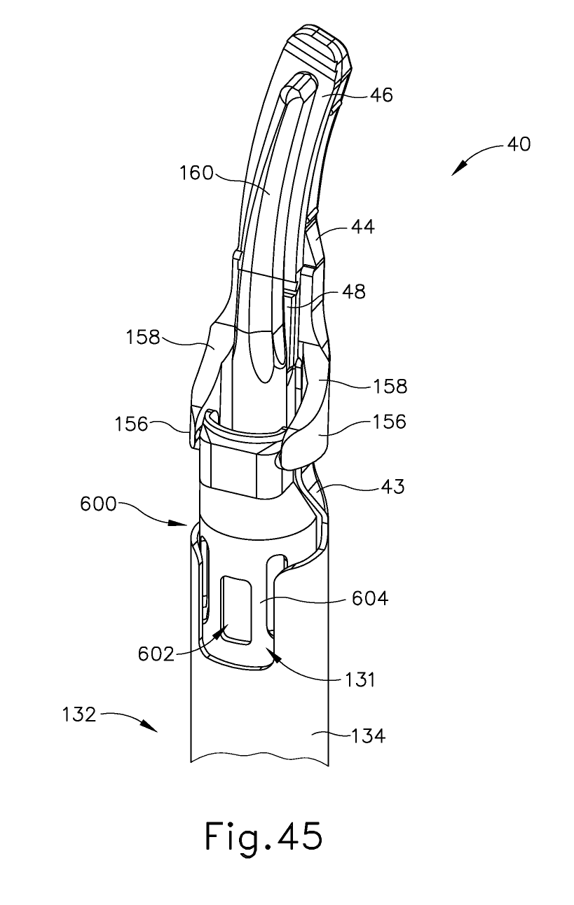

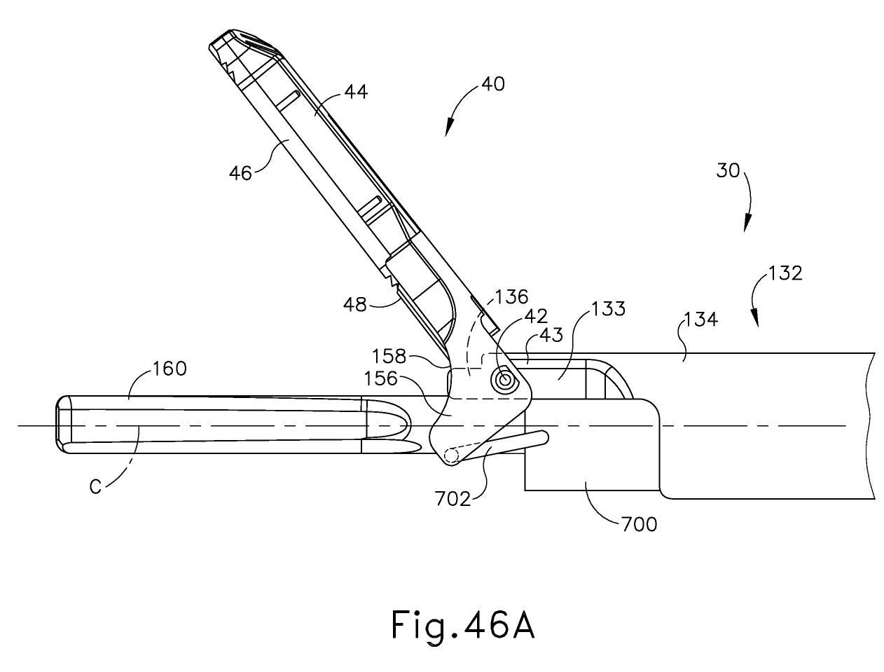

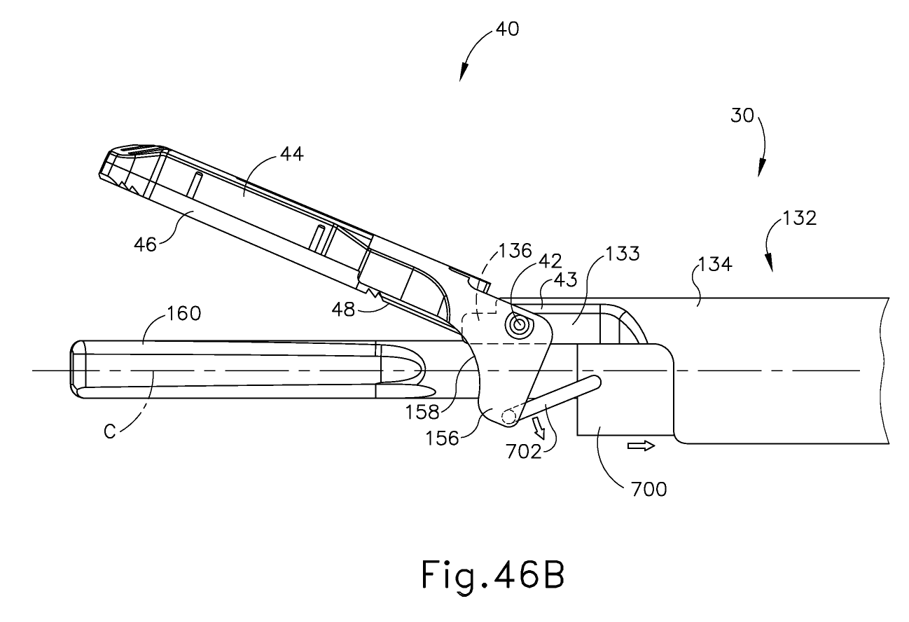

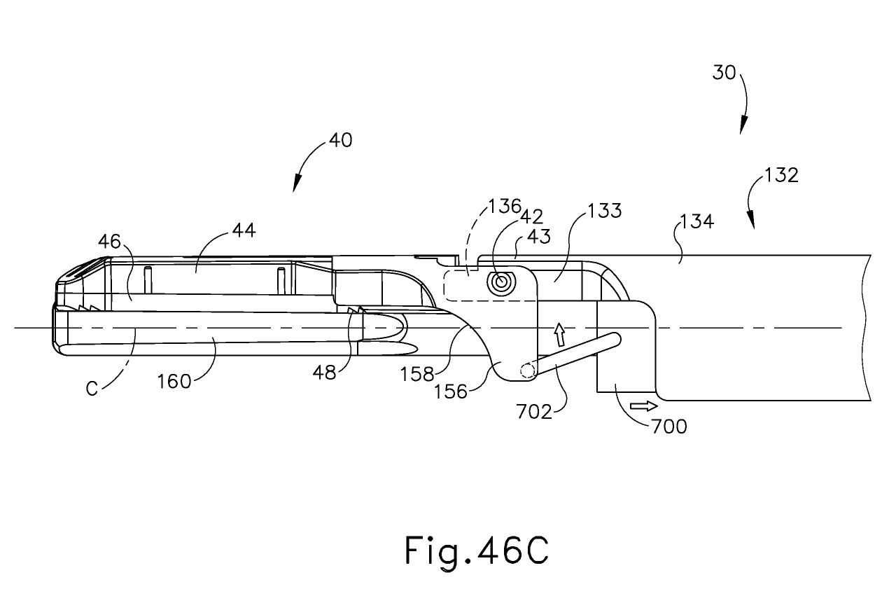

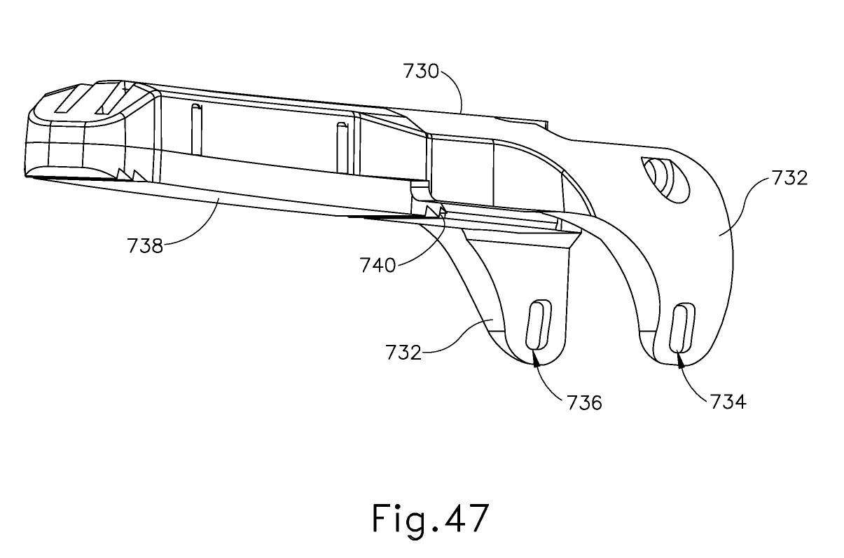

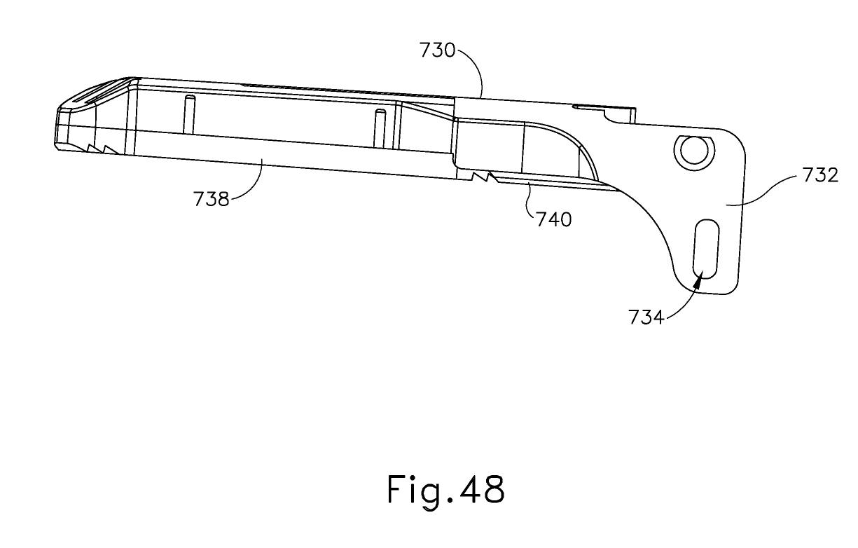

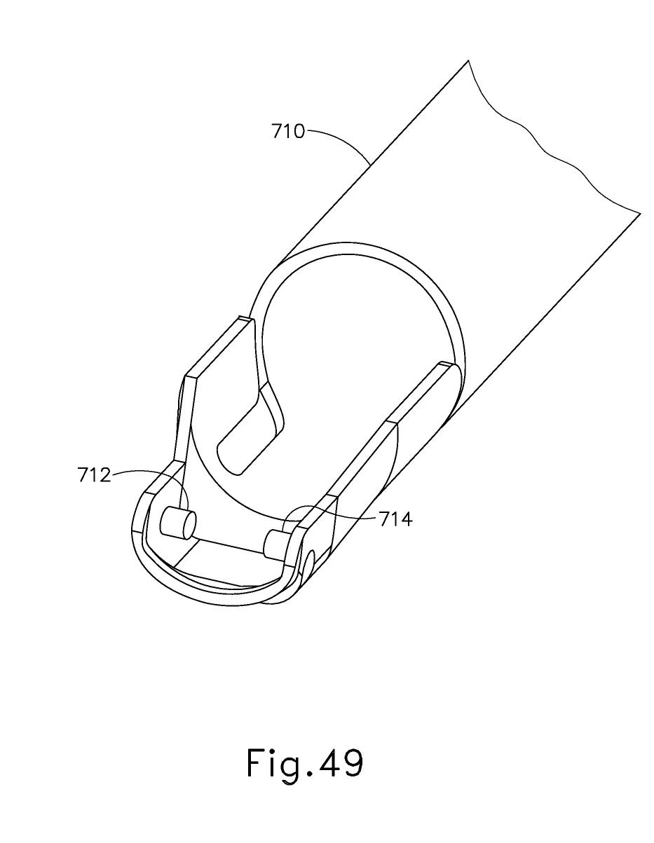

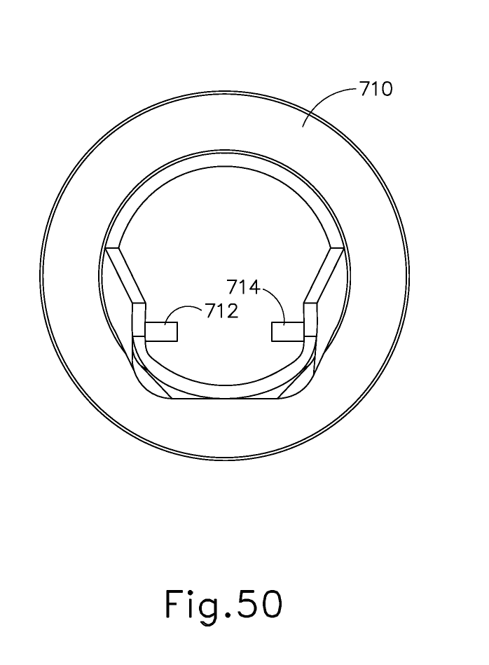

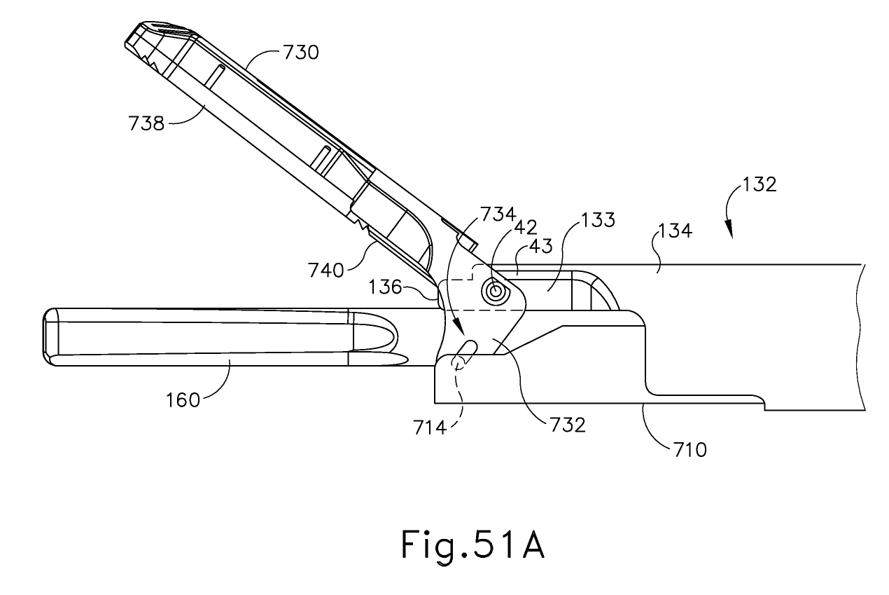

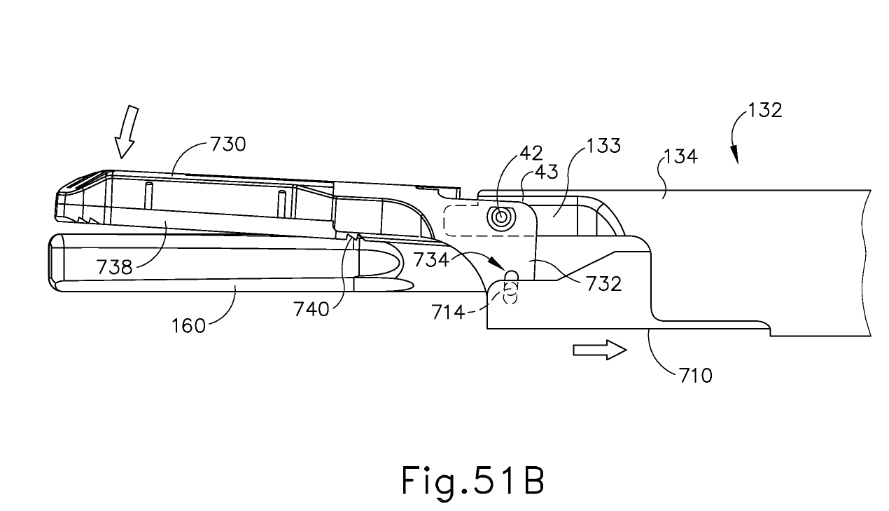

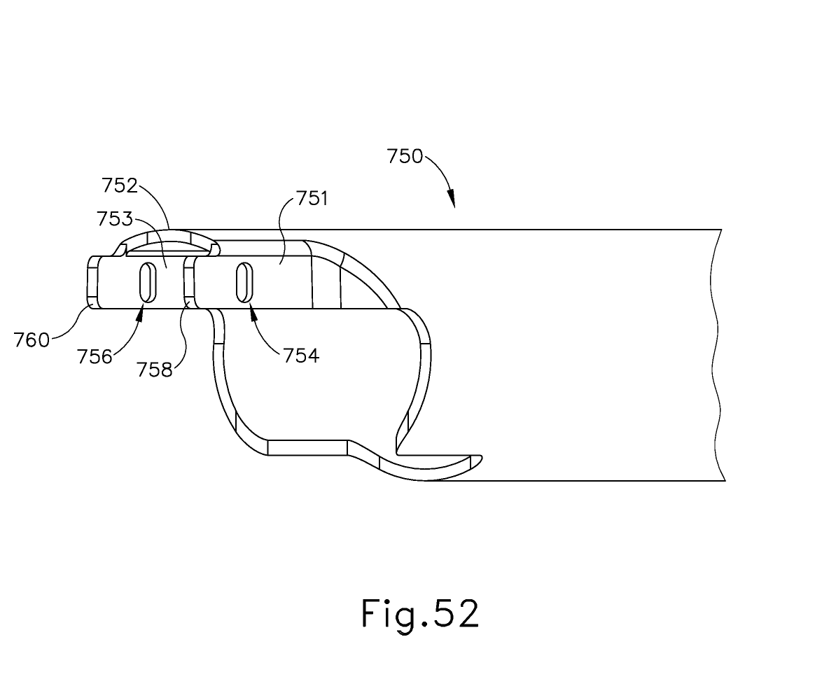

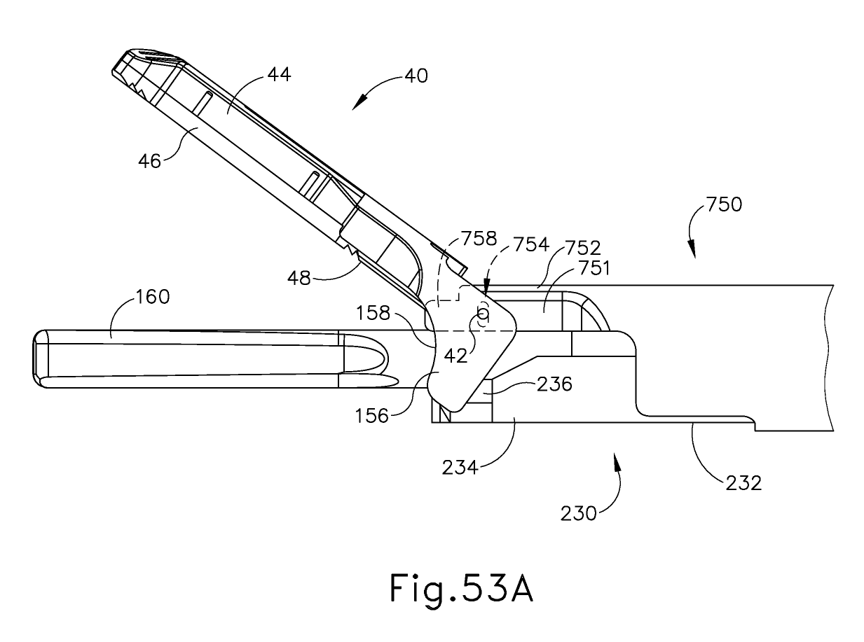

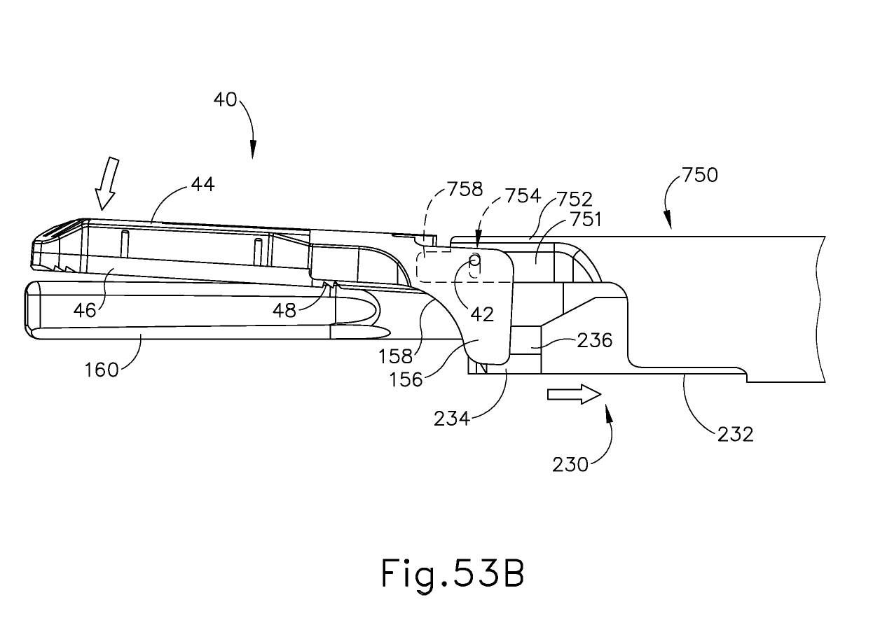

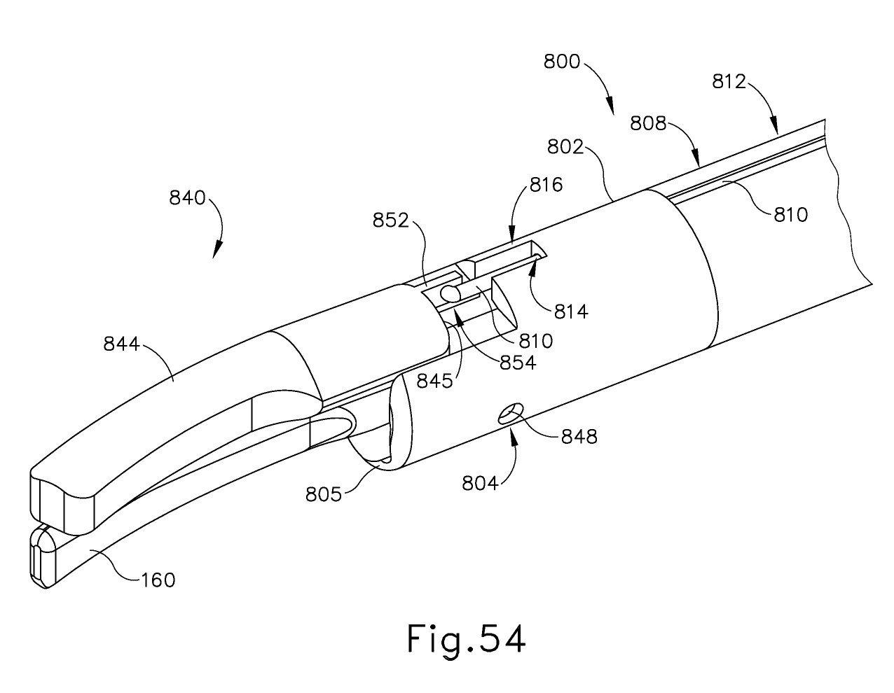

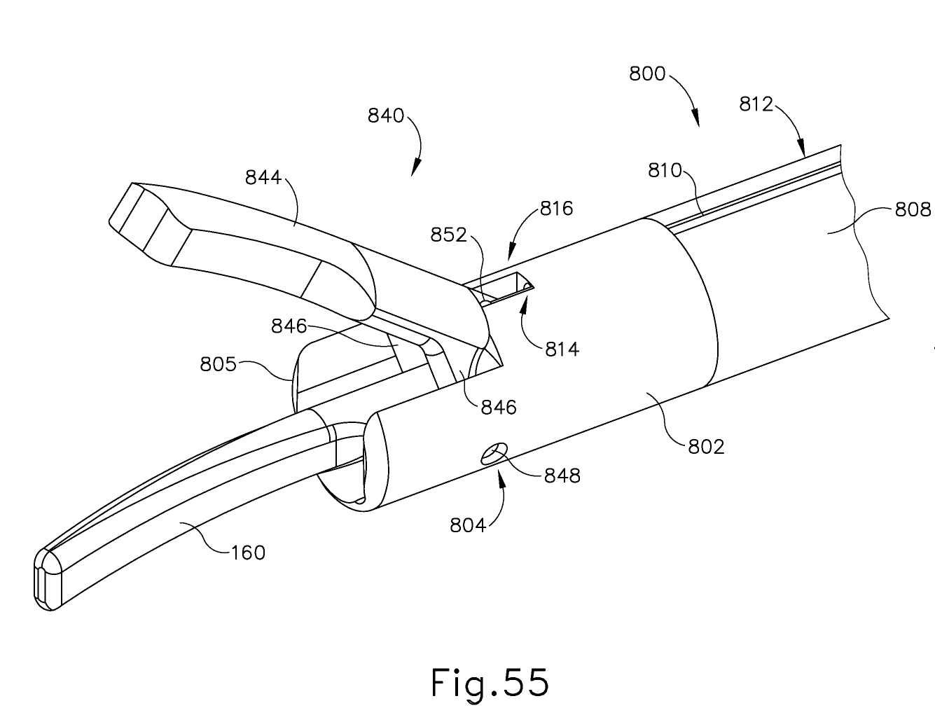

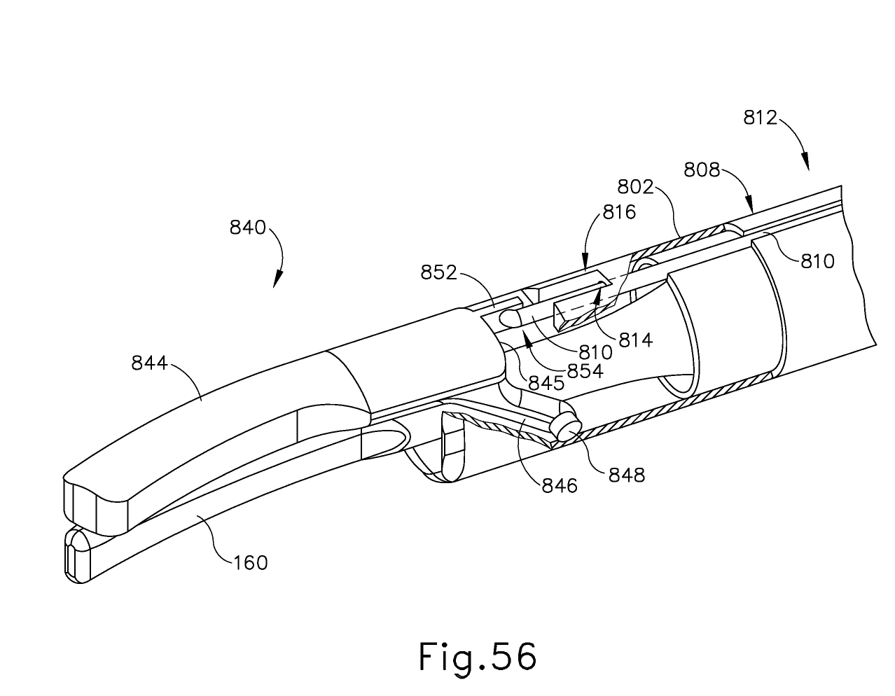

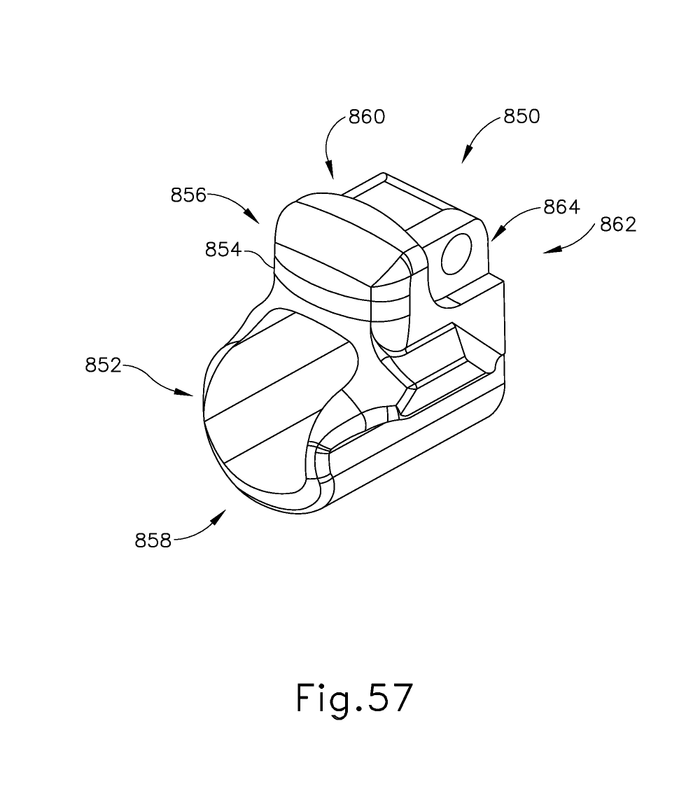

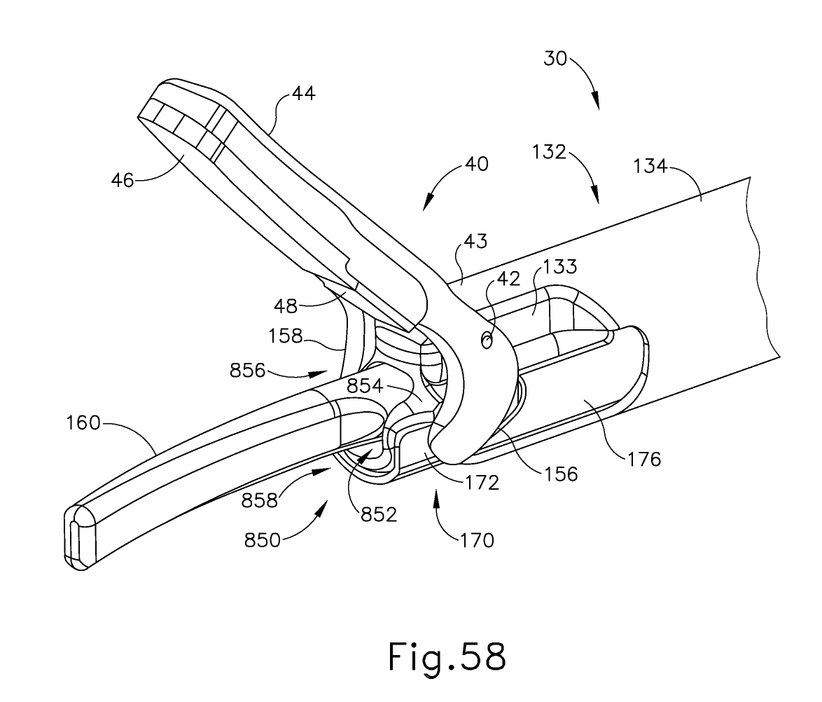

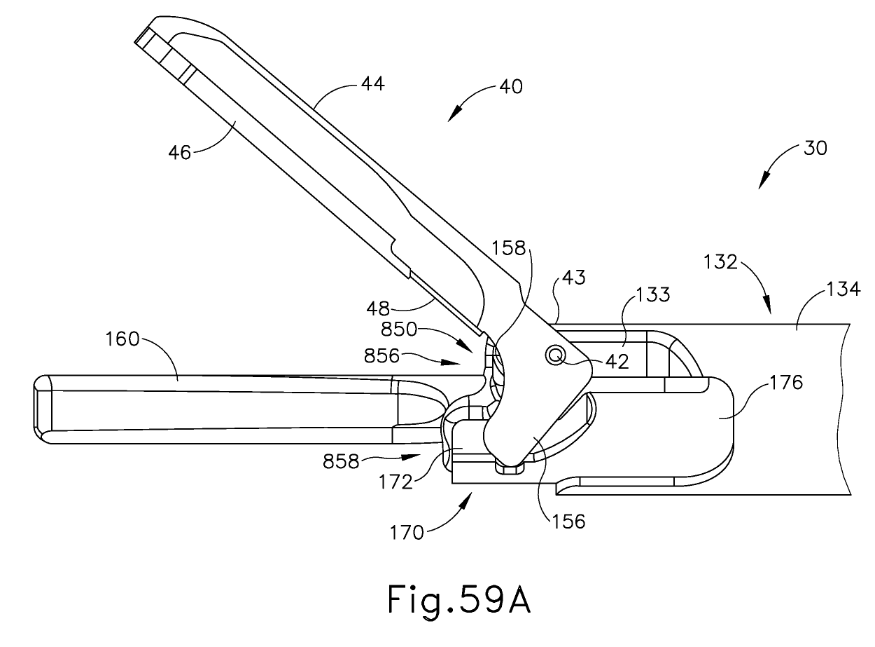

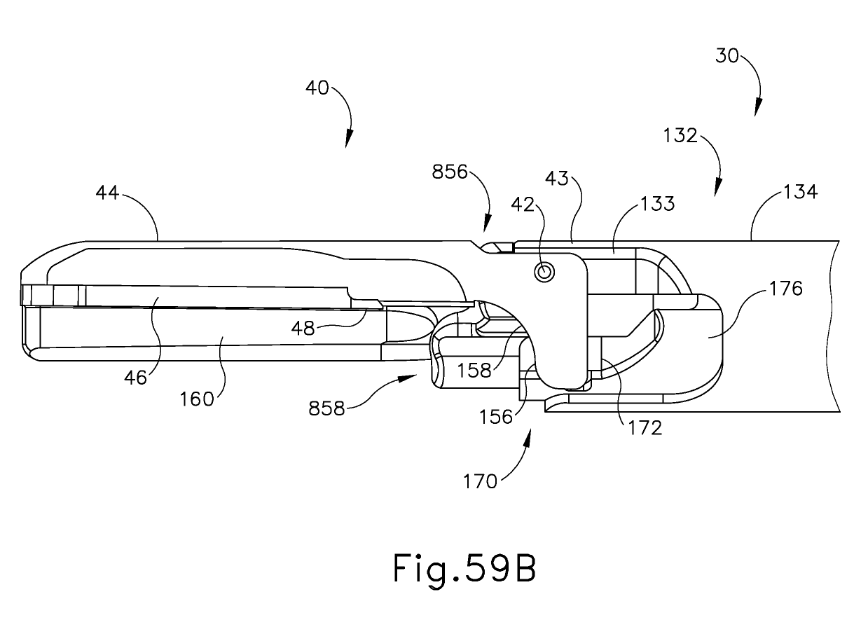

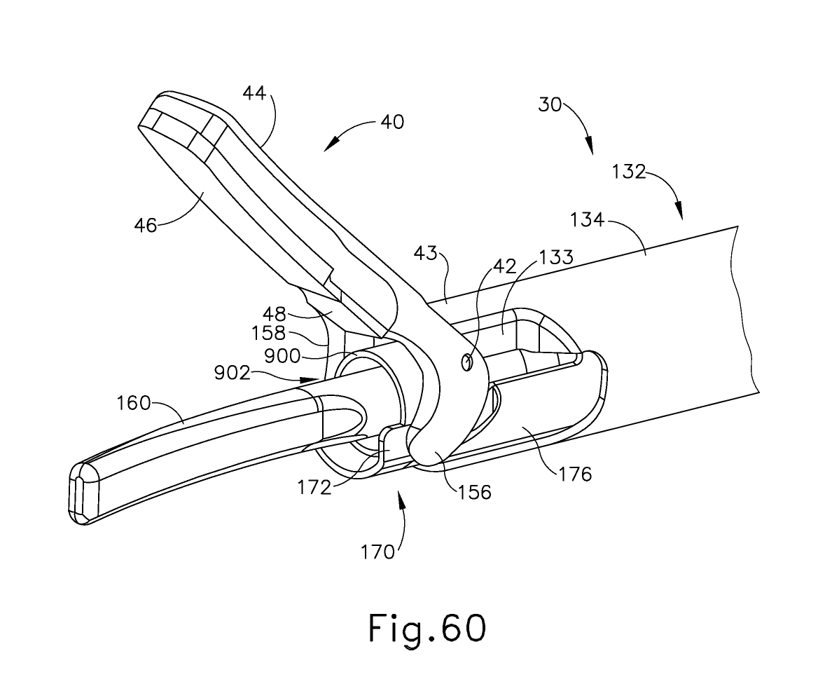

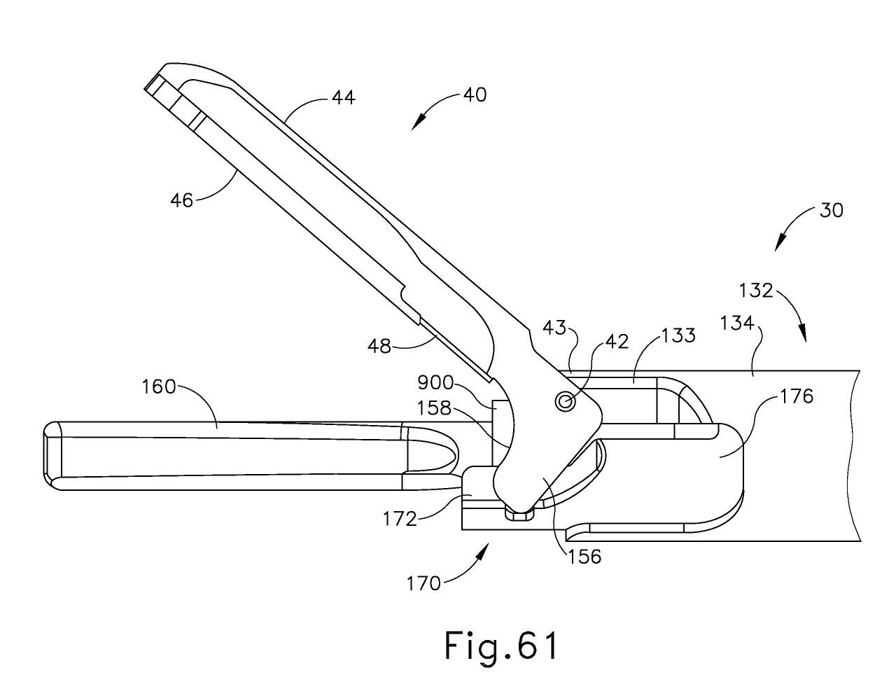

FIG. 2 with the clamp arm of FIG. 3 moved into a second rotational position by movement of the inner tube of FIG. 15A into a second longitudinal position, with the outer sheath of FIG. 14 driven into a flexed position;

FIG. 15C depicts a side elevational view of the end effector and shaft assembly of FIG. 2 with the clamp arm of FIG. 3 moved into a third rotational position by movement of the inner tube of FIG. 15A into a third longitudinal position;

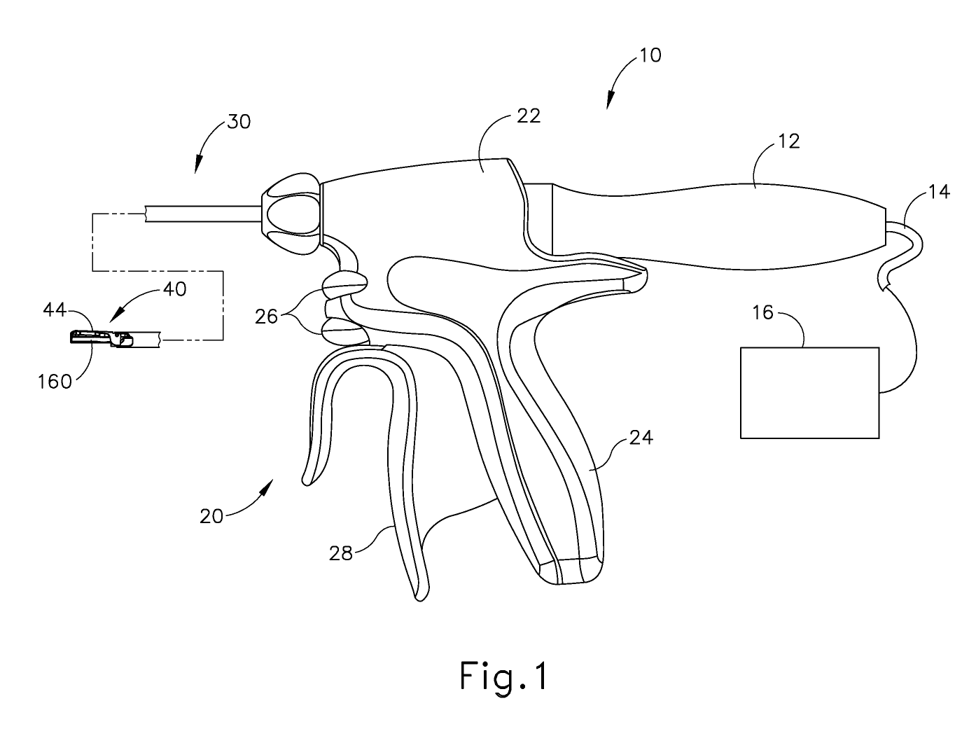

FIG. 16A depicts a side elevational view of the distal end of another exemplary alternative outer sheath configured for use with the instrument of FIG. 1, with a pivot member in a first rotational position;

FIG. 16B depicts a side elevational view of the distal end of the outer sheath of FIG. 16A, with the pivot member moved into a second rotational position;

FIG. 17A depicts a side elevational view of the distal end of another exemplary alternative outer sheath configured for use with the instrument of FIG. 1, with a pivot member in a first rotational position;

FIG. 17B depicts a side elevational view of the distal end of the outer sheath of FIG. 17A, with the pivot member moved into a second rotational position;

FIG. 18A depicts a side elevational view of the distal end of another exemplary alternative outer sheath configured for use with the instrument of FIG. 1, with a pivot member in a first rotational position;

FIG. 18B depicts a side elevational view of the distal end of the outer sheath of FIG. 18A, with the pivot member moved into a second rotational position;

FIG. 19 depicts a perspective view of an exemplary rotating pin;

FIG. 20 depicts a top view of the end effector of FIG. 2 having the pin of FIG. 19;

FIG. 21A depicts a side elevational view of the end effector and shaft assembly of FIG. 2 having the pin of FIG. 19, with the clamp arm of FIG. 3 in a first rotational position, and with the inner tube of FIG. 15A in a first longitudinal position;

FIG. 21B depicts a side elevational view of the end effector and shaft assembly of FIG. 2 with the clamp arm of FIG. 21A moved into a second rotational position by movement of the inner tube of FIG. 15A into a second longitudinal position;

FIG. 22 depicts a perspective view of an exemplary alternative rotating pin;

FIG. 23 depicts a top view of the end effector of FIG. 2 having the pin of FIG. 22;

FIG. 24A depicts a side elevational view of the end effector and shaft assembly of

FIG. 2 having the pin of FIG. 22 with the clamp arm of FIG. 3 in a first rotational position, and with the inner tube of FIG. 15A in a first longitudinal position;

FIG. 24B depicts a side elevational view of the end effector and shaft assembly of FIG. 2 with the clamp arm of FIG. 3 moved into a second rotational position by movement of the inner tube of FIG. 15A into a second longitudinal position;

FIG. 24C depicts a side elevational view of the end effector and shaft assembly of FIG. 2 with the clamp arm of FIG. 3 moved into a third rotational position by movement of the inner tube of FIG. 15A into a third longitudinal position;

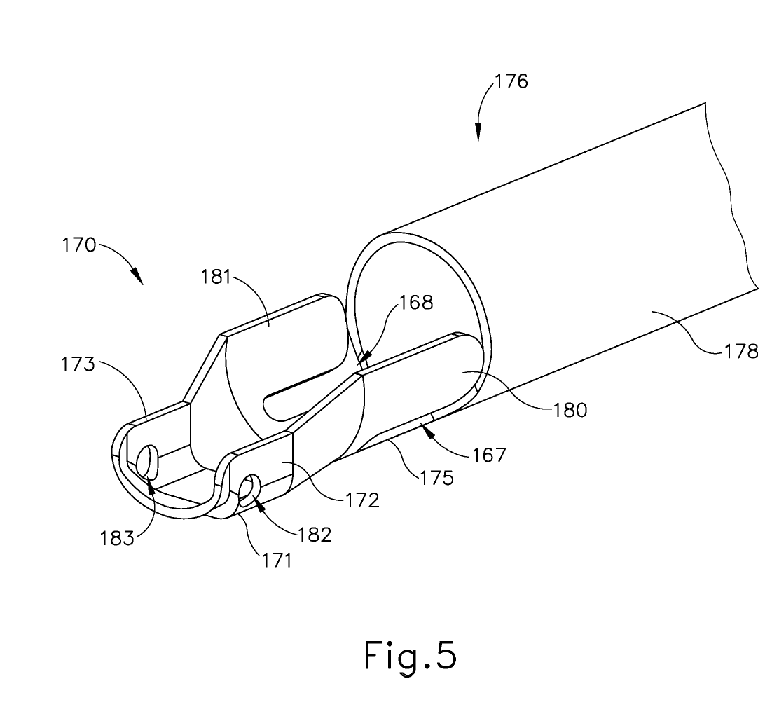

FIG. 25A depicts a side elevational view of the end effector and shaft assembly of FIG. 2 with the clamp arm of FIG. 3 in a first rotational position and with the inner tube of FIG. 5 in a first longitudinal position;

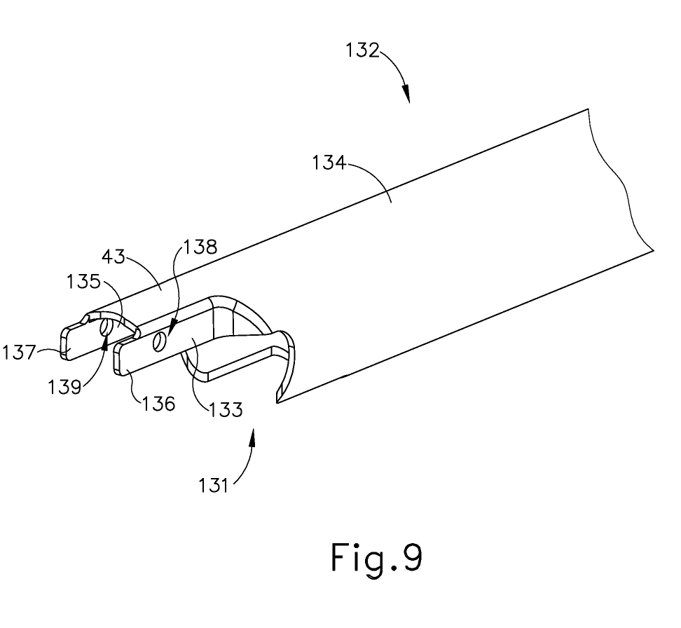

FIG. 25B depicts a side elevational view of the end effector and shaft assembly of FIG. 2 with the clamp arm of FIG. 3 moved into a second rotational position, with the inner tube of FIG. 5 moved into a second longitudinal position and driven into a flexed position such that the inner tube is engaged with the outer sheath of FIG. 9 such that engagement between the inner tube and the outer sheath restricts hyperextension of the clamp arm;

FIG. 26 depicts a top view of the end effector and shaft assembly of FIG. 2 with the clamp arm of FIG. 3 in the second rotational position, with the inner tube of FIG. 5 in the second longitudinal position and in the flexed position such that engagement between the inner tube and the outer sheath of FIG. 9 restricts hyperextension of the clamp arm;

FIG. 27A depicts a perspective view of the end effector and shaft assembly of FIG. 2 with an exemplary alternative outer sheath, with the clamp arm of FIG. 3 in a first rotational position, and with the inner tube of FIG. 5 in a first longitudinal position;

FIG. 27B depicts perspective view of the end effector and shaft assembly of FIG. 2 with the clamp arm of FIG. 3 moved into a second rotational position, with the inner tube of FIG. 5 moved into a second longitudinal position, and with tissue stops of the outer sheath of FIG. 27A driven into a bent position such that the tissue stops restrict hyperextension of the clamp arm;

FIG. 28 depicts a top view of the end effector and shaft assembly of FIG. 2 with the clamp arm of FIG. 3 in the second rotational position, with the inner tube of FIG. 5 in the second longitudinal position, and with the tissue stops of FIG. 27B in the bent position such that the tissue stops prevent rotation of the clamp arm;

FIG. 29A depicts a cross-sectional side view of the shaft assembly of FIG. 2 having an exemplary alternative outer sheath and inner tube in a first longitudinal position relative to the outer sheath, with a locking tab of the inner tube in a first rotational position;

FIG. 29B depicts a cross-sectional side view of the shaft assembly of FIG. 2 with the inner tube of FIG. 29A moved into second longitudinal position relative to the outer sheath of FIG. 29A, with the locking tab of the inner tube in a second rotational position;

FIG. 30 depicts a perspective view of the distal end of another exemplary alternative inner tube configured for use with the instrument of FIG. 1;

FIG. 31A depicts a side elevational view of the distal end of the inner tube of FIG. 30 with a distal portion of the inner tube in a first rotational position;

FIG. 31B depicts a side elevational view of the distal end of the inner tube of FIG. 30 with the distal portion of the inner tube moved into a second rotational position;

FIG. 32 depicts a perspective view of the distal end of yet another exemplary alternative inner tube;

FIG. 33A depicts a cross-sectional view of the shaft assembly of FIG. 2 with the inner tube of FIG. 32;

FIG. 33B depicts a cross-sectional view of the shaft assembly of FIG. 2 with the inner tube of FIG. 32 moved into a flexed position;

FIG. 34 depicts a perspective view of the distal end of yet another exemplary alternative inner tube;

FIG. 35A depicts a cross-sectional view of the shaft assembly of FIG. 2 with the inner tube of FIG. 34;

FIG. 35B depicts a cross-sectional view of the shaft assembly of FIG. 2 with the inner tube of FIG. 34 moved into a flexed position;

FIG. 36A depicts a cross-sectional view of the end effector and shaft assembly of

FIG. 2 with yet another exemplary alternative clamp arm configured for use with the instrument of FIG. 1 in a first rotational position;

FIG. 36B depicts a cross-sectional view of the end effector and shaft assembly of FIG. 2 with the clamp arm of FIG. 36A moved into a second rotational position such that the clamp arm engages a stop tab of yet another exemplary alternative outer sheath configured for use with the instrument of FIG. 1;

FIG. 37 depicts a perspective view of the distal end of an inner tube assembly configured for use with the instrument of FIG. 1;

FIG. 38 depicts a perspective view of a flex portion of the inner tube assembly of FIG. 37;

FIG. 39 depicts a perspective view of the distal end of a tube of the inner tube assembly of FIG. 37;

FIG. 40 depicts a side elevational view of the distal end of the inner tube assembly of FIG. 37;

FIG. 41 depicts a perspective view of the end effector and shaft assembly of FIG. 2 with yet another exemplary alternative inner tube and outer sheath;

FIG. 42A depicts a side elevational view of the inner tube of FIG. 41 in a first longitudinal position relative to the outer sheath to FIG. 41;

FIG. 42B depicts a side elevational view of the inner tube of FIG. 41 moved into a second longitudinal position relative to the outer sheath of FIG. 41 such that features of the inner tube engage features of the outer sheath to provide audible and/or tactile feedback;

FIG. 43 depicts a bottom view of the distal end of yet another exemplary alternative inner tube configured for use with the instrument of FIG. 1;

FIG. 44 depicts a perspective view of the distal end of the inner tube of FIG. 43;

FIG. 45 depicts a perspective view of the distal end of the shaft assembly of FIG. 2 with the inner tube of FIG. 43;

FIG. 46A depicts a side elevational view of an exemplary alternative end effector and shaft assembly with a clamp arm in a first rotational position and with an inner tube in a first longitudinal position;

FIG. 46B depicts a side elevational view of the end effector and shaft assembly of

FIG. 46A with the clamp arm moved to a second rotational position by movement of the inner tube to a second longitudinal position;

FIG. 46C depicts a side elevational view of the end effector and shaft assembly of

FIG. 46A with the clamp arm moved to a third rotational position by movement of the inner tube to a third longitudinal position;

FIG. 47 depicts a perspective view of an exemplary alternative clamp arm;

FIG. 48 depicts a side elevational view of the clamp arm of FIG. 47;

FIG. 49 depicts a perspective view of another exemplary alternative inner tube;

FIG. 50 depicts a front elevational view of the inner tube of FIG. 49;

FIG. 51A depicts a side elevational view of the end effector and shaft assembly of FIG. 2 with the clamp arm of FIG. 47 and the inner tube of FIG. 49, with the clamp arm in a first rotational position and with the inner tube in a first longitudinal position;

FIG. 51B depicts a side elevational view of the end effector and shaft assembly of FIG. 2 with the clamp arm of FIG. 47 and the inner tube of FIG. 49, with the clamp arm moved to a second rotational position by movement of the inner tube to a second longitudinal position;

FIG. 52 depicts a detailed perspective view of another exemplary alternative outer sheath;

FIG. 53A depicts a side elevational view of the end effector and shaft assembly of FIG. 2 with the outer sheath of FIG. 52 and the inner tube of FIG. 15A, with the clamp arm in a first rotational position and with the inner tube in a first longitudinal position;

FIG. 53B depicts a side elevational view of the end effector and shaft assembly of FIG. 2 with the outer sheath of FIG. 52 and the inner tube of FIG. 15A, with the clamp arm moved to a second rotational position by movement of the inner tube to a second longitudinal position;

FIG. 54 depicts a detailed perspective view of yet another exemplary alternative end effector and shaft assembly with a clamp arm in a closed position;

FIG. 55 depicts a detailed perspective view of the end effector and shaft assembly of FIG. 54 with the clamp arm in an open position;

FIG. 56 depicts a detailed perspective view of the end effector and shaft assembly of FIG. 54 with the clamp arm in the closed position, and with a collar of the shaft shown transparently to reveal internal details;

FIG. 57 depicts a perspective view of an exemplary tissue stop insert;

FIG. 58 depicts a detailed perspective view of the end effector and shaft assembly of FIG. 2 with the clamp arm in an open position, and with the tissue stop insert of FIG. 57 positioned within the shaft assembly;

FIG. 59A depicts a side elevational view of the end effector and shaft assembly of FIG. 2 with the clamp arm in the open position, and with the tissue stop insert of FIG. 57 positioned within the shaft assembly;

FIG. 59B depicts a side elevational view of the end effector and shaft assembly of FIG. 2 with the clamp arm moved into a closed position, and with the tissue stop insert of FIG. 57 positioned within the shaft assembly;

FIG. 60 depicts a detailed perspective view of the end effector and shaft assembly of FIG. 2 with the clamp arm in an open position, and with an exemplary tissue stop tube positioned within the shaft assembly; and

FIG. 61 depicts a side elevational view of the end effector and shaft assembly of FIG. 2 with the clamp arm in an open position, and with the tissue stop tube of FIG. 60 positioned within the shaft assembly.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument.

### I. EXEMPLARY ULTRASONIC SURGICAL INSTRUMENT

FIG. 1 illustrates an exemplary ultrasonic surgical instrument (10). At least part of instrument (10) may be constructed and operable in accordance with at least some of the teachings of U.S. Pat. No. 5,322,055; U.S. Pat. No. 5,873,873; U.S. Pat. No. 5,980,510; U.S. Pat. No. 6,325,811; U.S. Pat. No. 6,773,444; U.S. Pat. No. 6,783,524; U.S. Pub. No. 2006/0079874; U.S. Pub. No. 2007/0191713; U.S. Pub. No. 2007/0282333; U.S. Pub. No. 2008/0200940; U.S. Pub. No. 2009/0105750; U.S. Pub. No. 2010/0069940; U.S. Pub. No. 2011/0015660; U.S. Pub. No. 2012/0112687; U.S. Pub. No. 2012/0116265; U.S. patent application Ser. No. 13/538,588; U.S. patent application Ser. No. 13/657,553; U.S. Pat. App. No. 61/410,603; and/or U.S. patent application Ser. No. 14/028,717. The disclosures of each of the foregoing patents, publications, and applications are incorporated by reference herein. As described therein and as will be described in greater detail below, instrument (10) is operable to cut tissue and seal or weld tissue (e.g., a blood vessel, etc.) substantially simultaneously. It should also be understood that instrument (10) may have various structural and functional similarities with the HARMONIC ACE® Ultrasonic Shears, the HARMONIC WAVE® Ultrasonic Shears, the HARMONIC FOCUS® Ultrasonic Shears, and/or the HARMONIC SYNERGY® Ultrasonic Blades. Furthermore, instrument (10) may have various structural and functional similarities with the devices taught in any of the other references that are cited and incorporated by reference herein.

To the extent that there is some degree of overlap between the teachings of the references cited herein, the HARMONIC ACE® Ultrasonic Shears, the HARMONIC WAVE® Ultrasonic Shears, the HARMONIC FOCUS® Ultrasonic Shears, and/or the HARMONIC SYNERGY® Ultrasonic Blades, and the following teachings relating to instrument (10), there is no intent for any of the description herein to be presumed as admitted prior art. Several teachings herein will in fact go beyond the scope of the teachings of the references cited herein and the HARMONIC ACE® Ultrasonic Shears, the HARMONIC WAVE® Ultrasonic Shears, the HARMONIC FOCUS® Ultrasonic Shears, and the HARMONIC SYNERGY® Ultrasonic Blades.

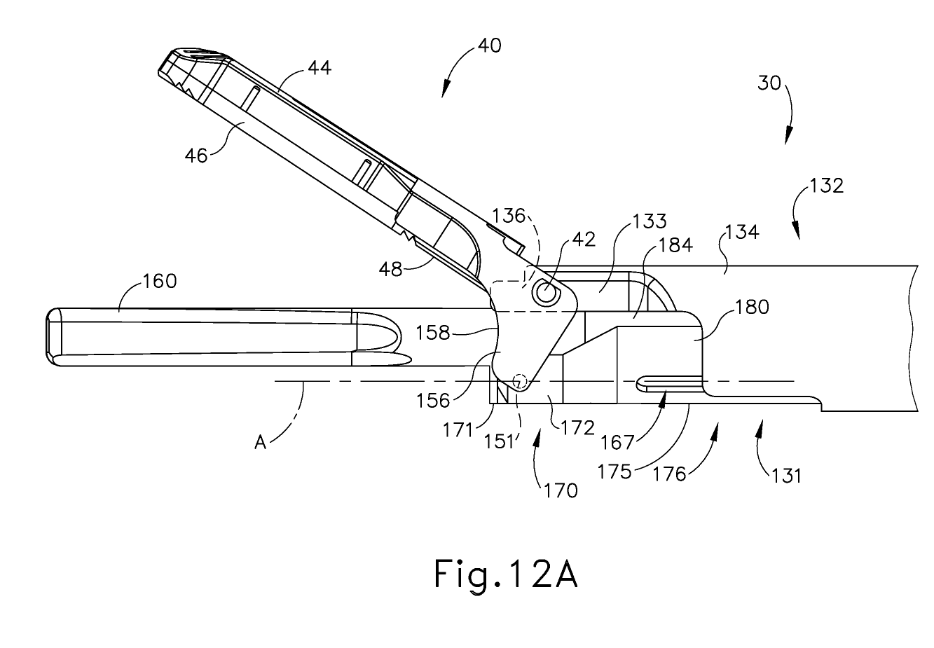

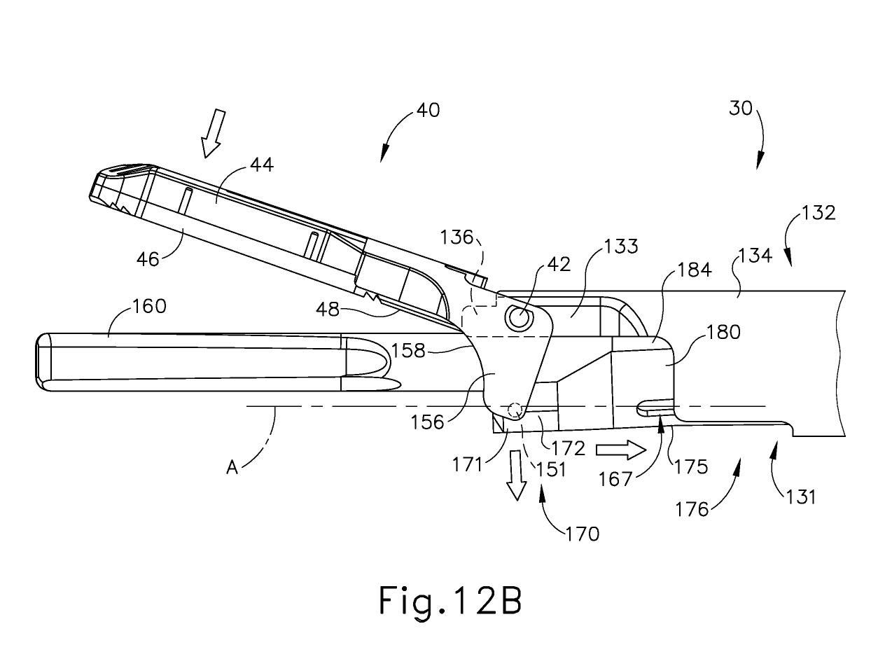

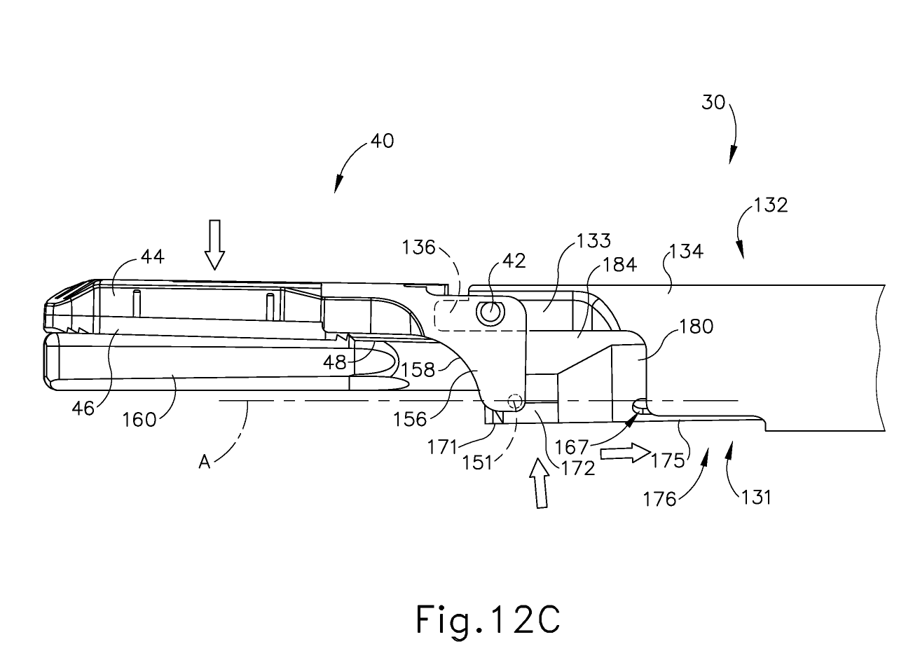

Instrument (10) of the present example comprises a handle assembly (20), a shaft assembly (30), and an end effector (40). Handle assembly (20) comprises a body (22) including a pistol grip (24) and a pair of buttons (26). Handle assembly (20) also includes a trigger (28) that is pivotable toward and away from pistol grip (24). It should be understood, however, that various other suitable configurations may be used, including but not limited to a scissor grip configuration. End effector (40) includes an ultrasonic blade (160) and a pivoting clamp arm (44). Clamp arm (44) is coupled with trigger (28) such that clamp arm (44) is pivotable toward ultrasonic blade (160) in response to pivoting of trigger (28) toward pistol grip (24); and such that clamp arm (44) is pivotable away from ultrasonic blade (160) in response to pivoting of trigger (28) away from pistol grip (24). Various suitable ways in which clamp arm (44) may be coupled with trigger (28) will be apparent to those of ordinary skill in the art in view of the teachings herein. In some versions, one or more resilient members are used to bias clamp arm (44) and/or trigger (28) to the open position shown in FIG. 12A.

An ultrasonic transducer assembly (12) extends proximally from body (22) of handle assembly (20). Transducer assembly (12) is coupled with a generator (16) via a cable (14). Transducer assembly (12) receives electrical power from generator (16) and converts that power into ultrasonic vibrations through piezoelectric principles. Generator (16) may include a power source and control module that is configured to provide a power profile to transducer assembly (12) that is particularly suited for the generation of ultrasonic vibrations through transducer assembly (12). By way of example only, generator (16) may comprise a GEN 300 sold by Ethicon Endo-Surgery, Inc. of Cincinnati, Ohio. In addition or in the alternative, generator (16) may be constructed in accordance with at least some of the teachings of U.S. Pub. No. 2011/0087212, entitled “Surgical Generator for Ultrasonic and Electrosurgical Devices,” published Apr. 14, 2011, the disclosure of which is incorporated by reference herein. It should also be understood that at least some of the functionality of generator (16) may be integrated into handle assembly (20), and that handle assembly (20) may even include a battery or other on-board power source such that cable (14) is omitted. Still other suitable forms that generator (16) may take, as well as various features and operabilities that generator (16) may provide, will be apparent to those of ordinary skill in the art in view of the teachings herein.

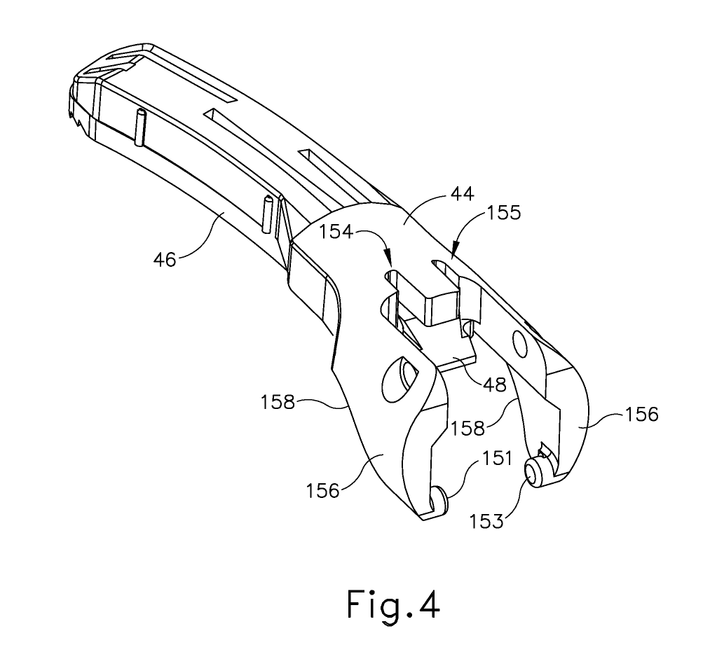

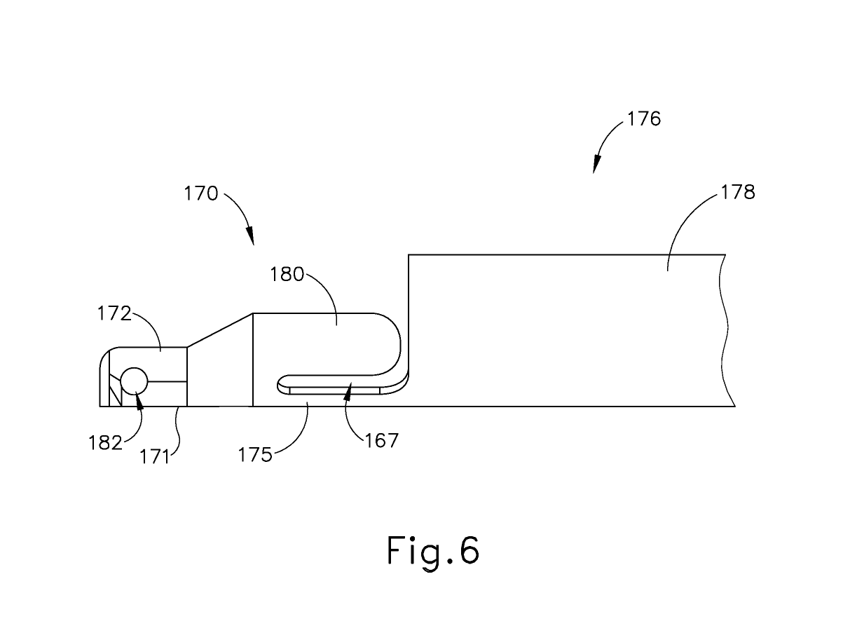

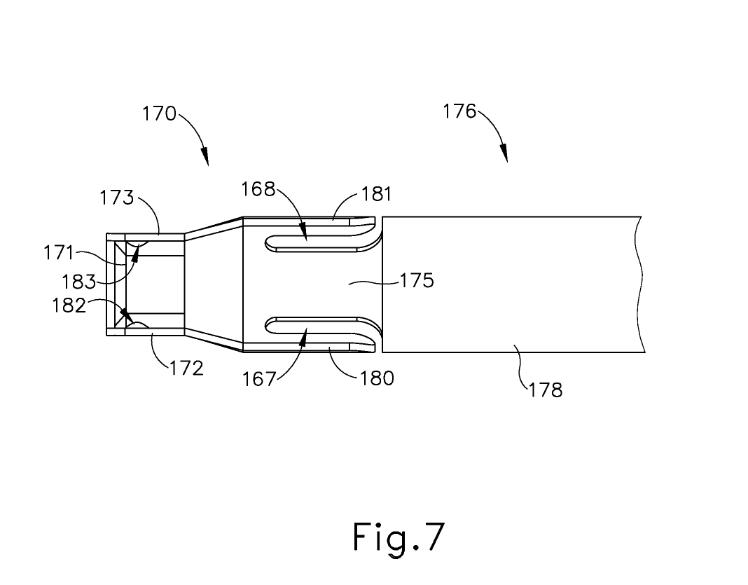

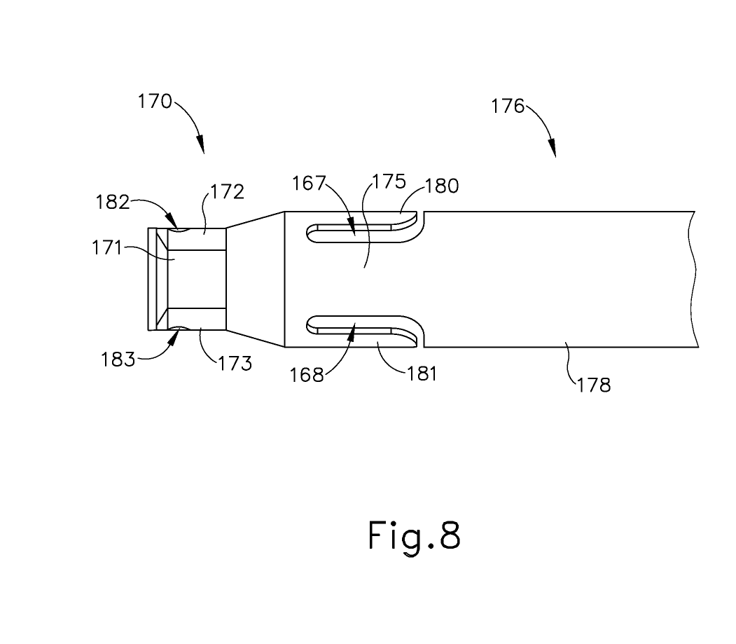

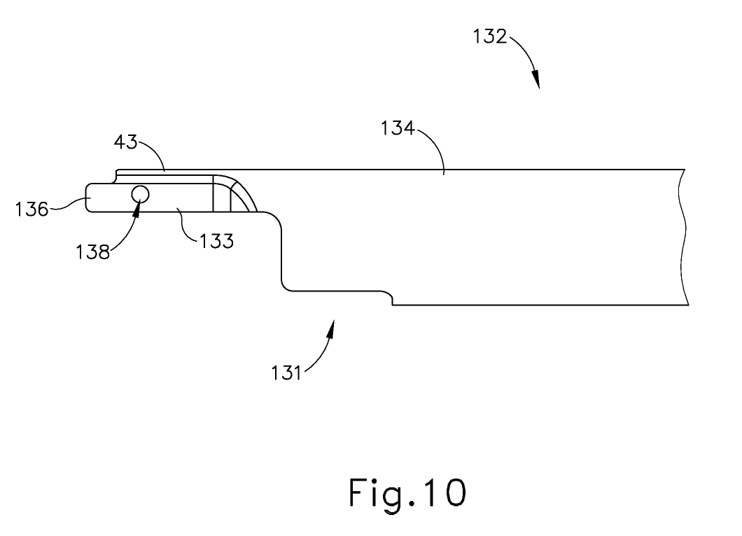

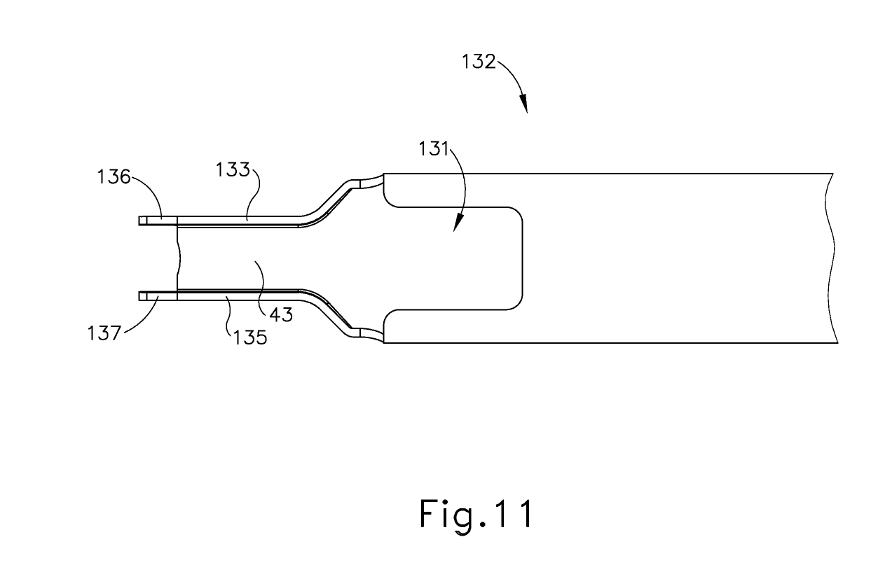

As best seen in FIGS. 2-13, end effector (40) of the present example comprises clamp arm (44) and ultrasonic blade (160). Clamp arm (44) includes a primary clamp pad (46) and a secondary clamp pad (48) that are secured to the underside of clamp arm (44), facing blade (160). Clamp arm (44) is pivotably secured to a distally projecting tongue (43) of an outer sheath (132) via a pin (42). Clamp arm (44) is operable to selectively pivot toward and away from blade (160) to selectively clamp tissue between clamp arm (44) and blade (160). A pair of arms (156) extend transversely from clamp arm (44) and are secured to a distal portion (170) of an inner tube (176) that extends laterally between arms (156). Arms (156) are secured to distal portion (170) via a pair of integral, inwardly extending pins (151, 153), which are rotatably disposed within a pair of circular through holes (182, 183) of distal portion (170). As best seen in FIG. 3, arms (156) comprise a concave surface (158). As will be discussed in more detail below, concave surface (158) allows for proximal movement of tissue between clamp arm (44) and ultrasonic blade (160). As best seen in FIG. 4, clamp arm (44) further comprises a pair of slots (154, 155) formed in a top surface of clamp arm (44). As will be discussed in more detail below, slots (154, 155) are configured to receive a pair of tissue stops (136, 137) to thereby permit complete closure of clamp arm (44) into a closed position. As will also be discussed in more detail below, tissue stops (136, 137) are configured to inhibit proximal movement of tissue beyond blade (160) and/or into the interior of outer sheath (132) and/or inner tube (176).

In the present example, each pin (151, 153) has a substantially circular cross-sectional profile. By way of example only, pins (151, 153) may be coined to have a round shape. By way of further example only, each pin may have a diameter in the range of approximately 0.027 inches to approximately 0.0305 inches. In some versions, holes (182, 183) are also circular, and each hole (182, 183) has a diameter in the range of approximately 0.032 inches to approximately 0.035 inches. Holes (182, 183) may provide a clearance for pins (151, 153) in the range of approximately 0.0015 inches to approximately 0.008 inches. Alternatively, any other suitable sizes or clearances may be provided. It should also be appreciated that pins (151, 153) may be replaced with a single pin extending between opposing interior surfaces of arms (156) of clamp arm (44). Such a pin may be welded in place, or secured to arms (156) in any other appropriate manner.

As shown in FIGS. 5-8, inner tube (176) comprises a rigid tubular portion (178) and a distal portion (170). Distal portion (170) is secured to rigid tubular portion (178) by a flexible portion (175). Flexible portion (175) is defined by a pair of slots (167, 168) formed within inner tube (176). Slots (167, 168) permit flexible movement of flexible portion (175) and further define a pair of “nacelle” flanges (180, 181) as will be discussed in more detail below. Flexible portion (175) is operable to provide selective positioning of distal portion (170) at various lateral deflection angles relative to a reference plane (A), which is parallel to a longitudinal axis defined by rigid tubular portion (178). As will be discussed in more detail below, distal portion (170) is operable to flex to provide for rotation of clamp arm (44). Distal portion (170) comprises a pair of flanges (172, 173) extending upwardly from a base (171). Each flange (171, 172) comprises a circular through hole (182, 183), as noted above, and a flange (180, 181) extending proximally from each flange (171, 172) respectively. As discussed above, clamp arm (44) is pivotably secured to flanges (171, 172) of distal portion (170) via a pair of inwardly extending pins (151, 153) of arms (156). Pins (151, 153) are rotatably disposed within through holes (182, 183). Inner tube (176) is operable to translate longitudinally within outer sheath (132) relative to outer sheath (132) to selectively pivot clamp arm (44) toward and away from blade (160). In particular, inner tube (176) is coupled with trigger (28) such that clamp arm (44) pivots toward blade (160) in response to pivoting of trigger (28) toward pistol grip (24); and such that clamp arm (44) pivots away from blade (160) in response to pivoting of trigger (28) away from pistol grip (24). Clamp arm (44) may be biased toward the open position, such that (at least in some instances) the operator may effectively open clamp arm (44) by releasing a grip on trigger (28).

As shown in FIGS. 9-11, outer sheath (132) comprises a rigid tubular portion (134) having a distally projecting rigid tongue (43) extending from a distal end of rigid tubular portion (134). Tongue (43) comprises a pair of flanges (133, 135). Each flange (133, 135) comprises a circular through hole (138, 139) and a tissue stop (136, 137) extending distally from each flange (133, 135) respectively. Clamp arm (44) is pivotably secured to tongue (43) of outer sheath (132) via a pin (42) rotatably disposed within through holes (138, 139). As mentioned above, and as will be discussed in more detail below, slots (154, 155) of clamp arm (44) are configured to slidably receive tissue stops (136, 137) to thereby permit complete closure of clamp arm (44) into the closed position as shown in FIG. 12C. Outer sheath (132) comprises a slot (131) formed in a bottom surface of rigid tubular portion (134). As will be discussed in more detail below, slot (131) is configured to accommodate downward deflection of distal portion (170) of inner tube (176) along a path that is transverse to the longitudinal axis of outer sheath (132).

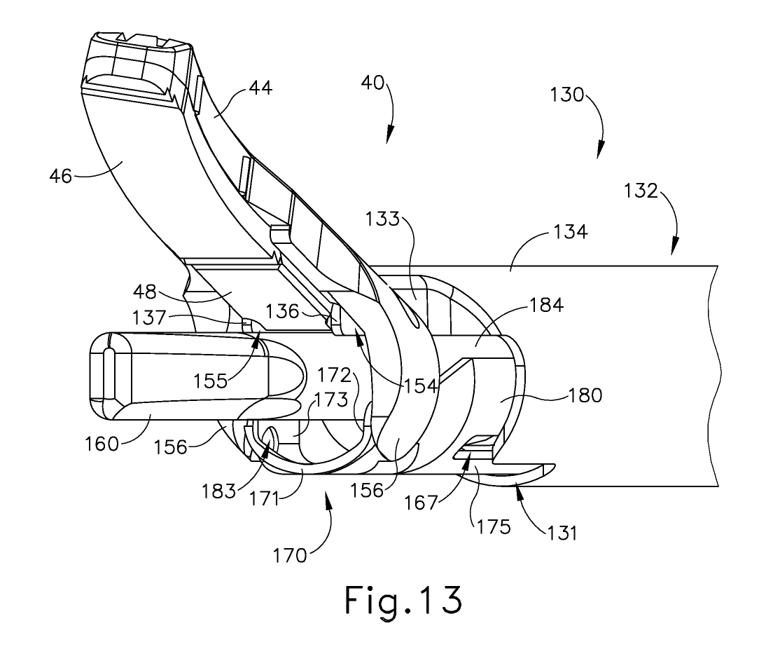

FIGS. 12A-13 show the operation of clamp arm (44) between an open position (FIG. 12A) and a closed position (FIG. 12C). As shown in FIG. 12A, when inner tube (176) is in a distal position relative to outer sheath (132), clamp arm (44) is in the open position. With clamp arm (44) in the open position, pin (42) (which pivotably couples clamp arm (44) with outer sheath (132)) is vertically offset relative to pins (151, 153) (which pivotably couple clamp arm (44) with inner tube (176)). At this stage, pins (151, 153) are positioned on reference plane (A) and distal portion (170) of inner tube (176) extends parallel to reference plane (A). Furthermore, as best seen in FIG. 12A and 13, with clamp arm (44) in the open position, the distal ends of tissue stops (136, 137) extend distally relative to concave surface (158) of clamp arm (44), to thereby inhibit proximal movement of tissue beyond blade (160). In other words, tissue stops (136, 137) extend distally relative to arms (156) of clamp arm (44), thereby serving as positive stops to restrict proximal migration of tissue beyond an operative surface of blade (160) at the proximal end of blade (160). At this stage, tissue stops (136, 137) also prevent tissue from reaching arms (156) of clamp arm (44) at regions where the tissue might otherwise be clamped between arms (156) and blade (160).

As shown in FIG. 12B, as inner tube (176) is moved proximally into an intermediate position, clamp arm (44) is pivoted toward blade (160) into an intermediate position. With clamp arm (44) in the intermediate position, pin (42) is substantially vertically aligned with pins (151, 153). Pins (151, 153) and distal portion (170) of inner tube (176) are deflected downwardly away from reference plane (A). Furthermore, with clamp arm (44) in the intermediate position, slots (154, 155) in clamp arm (44) begin to receive the distal ends of tissue stops (136, 137). The distal ends of tissue stops (136, 137) are still positioned substantially adjacent to concave surface (158) of clamp arm (44) to thereby inhibit proximal movement of tissue beyond an operative surface of blade (160) and to further prevent clamped tissue from reaching arms (156) of clamp arm (44) at regions where the tissue might otherwise be clamped between arms (156) and blade (160). Shortly after continuing past the stage shown in FIG. 12B, secondary clamp pad (48) begins to engage blade (160) and thereby inhibit proximal movement of tissue beyond an operative surface of blade (160). In other words, at an intermediate stage during the process of closing clamp arm (44), between the stage shown in FIG. 12B and the stage shown in FIG. 12C, the role of preventing proximal tissue migration is shifted from tissue stops (136, 137) to secondary clamp pad (48).

As shown in FIG. 12C, as inner tube (176) is moved further proximally into a proximal position, clamp arm (44) is pivoted toward blade (160) into the closed position. With clamp arm (44) in the closed position, pin (42) is no longer substantially vertically aligned with pins (151, 153). Pin (42) is instead vertically offset relative to pins (151, 153) such that, although pins (151, 153) and distal portion (170) of inner tube (176) remain in a deflected position, pins (151, 153) and distal portion (170) have moved back toward reference plane (A). (In some versions of instrument (10), pins (151, 153) and distal portion (170) may be returned into substantial alignment with reference plane (A) with clamp arm (44) in the closed position, as shown in FIG. 12A.) It should therefore be understood that the flexibility of distal portion (170) permits pins (151, 153) to travel along respective arcuate paths as clamp arm (44) pivots between the open position (FIG. 12A) and the closed position (FIG. 12C). With clamp arm (44) in the closed position, secondary clamp pad (48) continues to inhibit proximal movement of tissue beyond an operative surface of blade (160).

Blade (160) of the present example is operable to vibrate at ultrasonic frequencies in order to effectively cut through and seal tissue, particularly when the tissue is being clamped between clamp pad (46) and blade (160). Blade (160) is positioned at the distal end of an acoustic drivetrain. This acoustic drivetrain includes transducer assembly (12) and an acoustic waveguide (184). Transducer assembly (12) includes a set of piezoelectric discs (not shown) located proximal to a horn (not shown) of rigid acoustic waveguide (184). The piezoelectric discs are operable to convert electrical power into ultrasonic vibrations, which are then transmitted along acoustic waveguide (184) to blade (160) in accordance with known configurations and techniques. By way of example only, this portion of the acoustic drivetrain may be configured in accordance with various teachings of various references that are cited herein.

In the present example, the distal end of blade (160) is located at a position corresponding to an anti-node associated with resonant ultrasonic vibrations communicated through acoustic waveguide (184), in order to tune the acoustic assembly to a preferred resonant frequency fₒ when the acoustic assembly is not loaded by tissue. When transducer assembly (12) is energized, the distal end of blade (160) is configured to move longitudinally in the range of, for example, approximately 10 to 500 microns peak-to-peak, and in some instances in the range of about 20 to about 200 microns at a predetermined vibratory frequency fₒ of, for example, 55.5 kHz. When transducer assembly (12) of the present example is activated, these mechanical oscillations are transmitted through acoustic waveguide (184) to reach blade (160), thereby providing oscillation of blade (160) at the resonant ultrasonic frequency. Thus, when tissue is secured between blade (160) and clamp pads (46, 48), the ultrasonic oscillation of blade (160) may simultaneously sever the tissue and denature the proteins in adjacent tissue cells, thereby providing a coagulative effect with relatively little thermal spread. In some versions, an electrical current may also be provided through blade (160) and clamp arm (44) to also cauterize the tissue. While some configurations for an acoustic transmission assembly and transducer assembly (12) have been described, still other suitable configurations for an acoustic transmission assembly and transducer assembly (12) will be apparent to one or ordinary skill in the art in view of the teachings herein. Similarly, other suitable configurations for end effector (40) will be apparent to those of ordinary skill in the art in view of the teachings herein.

In some instances, as clamp arm (44) pivots toward blade (160) while tissue is interposed between clamp arm (44) and blade (160), the closing motion of clamp arm (44) may tend to drive the tissue proximally. As noted above, tissue stops (220, 222) are configured to restrict such proximal movement of tissue. In particular, tissue stops (220, 222) are configured to prevent tissue from migrating proximally to a point where the tissue would not be compressed between clamp pads (46, 48) and blade (160). Tissue stops (220, 222) thus ensure that the proximal-most regions of tissue between clamp arm (44) and blade (160) will be compressed between clamp pads (46, 48) and blade (160) during closure of clamp arm (44). Concave surfaces (158) of clamp arm (44) allow tissue stops (220, 222) to provide such tissue stopping capability. Concave surfaces (158) and tissue stops (220, 222) thus cooperate to prevent the occurrence of tissue “tags” (e.g., flattened but uncut regions of tissue) at the proximal end of end effector (40).

As also noted above, through holes (138, 139) of outer sheath (132) and through holes (182, 183) of inner tube (176) are circular in the present example. In addition, pins (42, 151, 153) all have circular profiles that complement the circular configuration of corresponding through holes (138, 139, 182, 183). It should be understood that the complementary circular configurations of pins (42, 151, 153) and through holes (138, 139, 182, 183) may make it relatively difficult to remove clamp arm (44) from end effector (40) (e.g., more difficult than would be the case where through holes (138, 139) and/or through holes (182, 183) are elongate in shape, etc.). Increasing the difficulty of removing clamp arm (44) from end effector (40) may decrease the occurrence of unauthorized reprocessing of clamp arm (44). Such unauthorized reprocessing may include unauthorized replacement of one or both of clamp pads (46, 48); or unauthorized replacement of the entire clamp arm (44).

It should also be understood that, during closure of clamp arm (44) toward blade (160), the complementary circular configurations of pins (42, 151, 153) and through holes (138, 139, 182, 183) may prevent the occurrence of “slop” or lost motion between inner tube (176) and clamp arm (44) that might otherwise occur in a conventional instrument where through holes (138, 139) and/or through holes (182, 183) are formed as elongate slots. The complementary circular configurations of pins (42, 151, 153) and through holes (138, 139, 182, 183) may also remove tolerance stackups that might otherwise occur in a conventional instrument where through holes (138, 139) and/or through holes (182, 183) are formed as elongate slots. Such removal of lost motion and/or tolerance stackups may provide a more consistent closure of clamp arm (44) toward blade (160) than might otherwise occur in a conventional instrument where through holes (138, 139) and/or through holes (182, 183) are formed as elongate slots. In other words, as clamp arm (44) is pivoted toward blade (160) repeatedly to transect and seal several regions of tissue, end effector (40) may provide a more consistent seal at each transection. End effector (40) may thus provide a more consistent and reliable performance than a conventional instrument where through holes (138, 139) and/or through holes (182, 183) are formed as elongate slots.

### II. EXEMPLARY ALTERNATIVE SHAFT ASSEMBLY FEATURES

It may be desirable to provide for flexibility within outer sheath (132) of instrument (10). It may additionally or alternatively be desirable to provide for rigidity within inner tube (176) of instrument (10). As will be discussed in more detail below, FIGS. 14-18B show various configurations through which flexibility may be provided to outer sheath (132) and/or rigidity may be provided to inner tube (176). While various examples by which flexibility may be provided to outer sheath (132) and/or rigidity may be provided to inner tube (176) will be described in greater detail below, other examples will be apparent to those of ordinary skill in the art in view of the teachings herein. It should be understood that the outer sheath and inner tube examples described below may function substantially similar to outer sheath (132) and inner tube (176) described above. In particular, clamp arm (44) may be rotatably coupled with both the outer sheath and the inner tube such that longitudinal translation of the inner tube relative to the outer sheath will selectively pivot clamp arm (44) toward and away from blade (160).

### A. EXEMPLARY OUTER SHEATH WITH FLEX SECTION

As shown in FIG. 14, an exemplary alternative outer sheath (200) comprises a rigid tubular portion (202) and a distal portion (204). Outer sheath (200) may be readily incorporated into instrument (10) discussed above. Distal portion (204) is secured to rigid tubular portion (202) via a flexible portion (206). Flexible portion (206) is defined by a slot (208) formed within outer sheath (200). Flexible portion (206) is operable to provide for selective positioning of distal portion (204) at various lateral deflection angles relative to a longitudinal axis defined by rigid tubular portion (202). As will be discussed on more detail below, distal portion (204) is operable to flex to provide for rotation of clamp arm (44). Distal portion (204) comprises a distally projecting rigid tongue (210). Tongue (210) comprises a pair of flanges (212, 214). Each flange (212, 214) comprises a circular through hole (216, 218) and a tissue stop (220, 222) extending distally from each flange (212, 214) respectively. Clamp arm (44) is pivotably secured to tongue (210) of outer sheath (200) via pin (42) rotatably disposed within through holes (216, 218). As will be discussed in more detail below, slots (154, 155) of clamp arm (44) are configured to slidably receive tissue stops (220, 222) to thereby permit complete closure of clamp arm (44) into the closed position as shown in FIG. 15C.

As shown in FIGS. 15A-15B, inner tube (230) of the present example comprises a rigid tubular portion (232) and a rigid distal portion (234). Inner tube (230) may be readily incorporated into instrument (10) discussed above. It should be understood that distal portion (234) is configured to be substantially inflexible relative to rigid tubular portion (232). Distal portion (234) comprises a pair of flanges (236, 238) extending from a base (240). Each flange (236, 238) comprises a circular through hole (not shown). As discussed above, clamp arm (44) is pivotably secured to flanges (236, 238) of distal portion (234) via inwardly extending pins (151, 153) of arms (156) rotatably disposed within the through holes. Inner tube (230) is operable to translate longitudinally within outer sheath (200) relative to outer sheath (200) to selectively pivot clamp arm (44) toward and away from blade (160). In particular, inner tube (230) is coupled with trigger (28) such that clamp arm (44) pivots toward blade (160) in response to pivoting of trigger (28) toward pistol grip (24); and such that clamp arm (44) pivots away from blade (160) in response to pivoting of trigger (28) away from pistol grip (24). Clamp arm (44) may be biased toward the open position, such that (at least in some instances) the operator may effectively open clamp arm (44) by releasing a grip on trigger (28).

FIGS. 15A-15C show the operation of clamp arm (44) between an open position (FIG. 15A) and a closed position (FIG. 15C). As shown in FIG. 15A, inner tube (230) is in a distal position relative to outer sheath (200), clamp arm (44) is in the open position. With clamp arm (44) in the open position, pin (42) (pivotably coupling clamp arm (44) with outer sheath (200)) is vertically offset relative to pins (151, 153) (pivotably coupling clamp arm (44) with inner tube (230)). This angular alignment provides for substantial alignment of distal portion (204) of outer sheath (200) with a reference plane (B), which is parallel with a longitudinal axis defined by rigid tubular portion (202) of outer sheath (200). Furthermore, with clamp arm (44) in the open position, the distal ends of tissue stops (220, 222) are positioned substantially adjacent to concave surface (158) of clamp arm (44) to thereby inhibit proximal movement of tissue beyond blade (160). It should be understood that tissue stops (220, 222) may have the same configuration and functionality as tissue stops (136, 137) described above.

As shown in FIG. 15B, as inner tube (230) is moved proximally into an intermediate position, clamp arm (44) is pivoted toward blade (160) into an intermediate position. With clamp arm (44) in the intermediate position, pin (42) is substantially vertically aligned with pins (151, 153). Pin (42) and distal portion (204) of inner tube (200) are deflected upwardly away from reference plane (B). Furthermore, with clamp arm (44) in the intermediate position and with distal portion (204) moved into the deflected position, slots (154, 155) in clamp arm (44) receive the distal ends of tissue stops (220, 222). The distal ends of tissue stops (220, 222) are still positioned substantially adjacent to concave surface (158) of clamp arm (44) to thereby inhibit proximal movement of tissue beyond blade (160).

As shown in FIG. 15C, as inner tube (230) is moved further proximally into a proximal position, clamp arm (44) is pivoted toward blade (160) into the closed position. With clamp arm (44) in the closed position, pin (42) is no longer substantially vertically aligned with pins (151, 153). Pin (42) is instead vertically offset relative to pins (151, 153) such that, although pin (42) and distal portion (204) of outer sheath (200) remain in a deflected position, pin (42) and distal portion (204) have moved toward reference plane (B). (In some versions, pin (42) distal portion (204) may be returned into substantial alignment with reference plane (B) with clamp arm (44) in the closed position, as shown in FIG. 15A.) Furthermore, at the stage shown in FIG. 15C, the distal ends of tissue stops (220, 222) remain positioned substantially adjacent to concave surface (158) of clamp arm (44) to thereby inhibit proximal movement of tissue beyond blade (160).

Although outer sheath (200) of the present example is described as being used with inner tube (230), it should be appreciated that outer sheath (200) may be used with inner tube (176) discussed above to thereby provide flexibility to both the outer sheath and the inner tube of shaft assembly (30).

### B. EXEMPLARY OUTER SHEATH WITH ROTATABLE DISTAL PORTION AND PIVOT PIN

FIGS. 16A and 16B show another exemplary alternative outer sheath (300) having a flexible distal portion (304). Outer sheath (300) may be readily incorporated into instrument (10) along with inner tube (230) discussed above. Outer sheath (300) comprises a rigid tubular portion (302) and a distal portion (304). Distal portion (304) is rotatably secured to rigid tubular portion (302) via a pin (306). In particular, pin (306) is rotatably disposed within a pair of flanges (308, 310) of distal portion (304) and a distal end of rigid tubular portion (302) such that distal portion (304) is operable to rotate about pin (306) relative to a longitudinal axis defined by rigid tubular portion (302). It should therefore be understood that distal portion (304) is operable to be selectively positioned at various lateral deflection angles relative to the longitudinal axis defined by rigid tubular portion (302). As should be understood from the discussion above, distal portion (304) is operable to rotate to provide for rotation of clamp arm (44). Distal portion (304) comprises a distally projecting rigid tongue (312). Rigid tongue (312) comprises a pair of flanges (314, 316). Each flange (314, 316) comprises a circular through hole (318, 320) and a tissue stop (322, 324) extending distally from each flange (314, 316) respectively. Clamp arm (44) is pivotably secured to rigid tongue (312) of outer sheath (300) via pin (42) rotatably disposed within through holes (318, 320). Slots (154, 155) of clamp arm (44) are configured to slidably receive tissue stops (322, 324) to thereby permit complete closure of clamp arm (44) into the closed position as discussed above.

As shown in FIG. 16B, rotation of distal portion (304) is limited by a projection (303) extending from a top surface of rigid tubular portion (302) which engages a proximal portion (305) of distal portion (304) as distal portion (304) is rotated away from the longitudinal axis defined by rigid tubular portion (302). This may prevent distal portion (304) from being intentionally or incidentally hyperextended (i.e., opened further past the proper open position, such as the position shown in FIG. 12A).

### C. EXEMPLARY OUTER SHEATH WITH ROTATABLE DISTAL PORTION AND PIVOT TAB

FIGS. 17A and 17B show yet another exemplary alternative outer sheath (330) having a flexible distal portion (334). Outer sheath (330) may be readily incorporated into instrument (10) along with inner tube (230) discussed above. Outer sheath (330) comprises a rigid tubular portion (332) and a distal portion (334). Distal portion (334) is rotatably secured to rigid tubular portion (332) via an oval-shaped tab (336) rotatably disposed within an oval-shaped opening (338). In particular, oval-shaped tab (336) is rotatably disposed within oval-shaped opening (338) such that distal portion (334) is operable to rotate about oval-shaped tab (336) relative to a longitudinal axis defined by rigid tubular portion (332). It should therefore be understood that distal portion (334) is operable to be selectively positioned at various lateral deflection angles relative to the longitudinal axis defined by rigid tubular portion (332). As should be understood from the discussion above, distal portion (334) is operable to rotate to provide for rotation of clamp arm (44). Distal portion (334) comprises a distally projecting rigid tongue (340). Rigid tongue (340) comprises a pair of flanges (342, 344). Each flange (342, 344) comprises a circular through hole (346, 348) and a tissue stop (350, 352) extending distally from each flange (342, 344) respectively. Clamp arm (44) is pivotably secured to rigid tongue (312) of outer sheath (330) via pin (42) rotatably disposed within through holes (346, 348). Slots (154, 155) of clamp arm (44) are configured to slidably receive tissue stops (350, 352) to thereby permit complete closure of clamp arm (44) into the closed position as discussed above.

As shown in FIG. 17B, rotation of distal portion (334) is limited by a distal portion (333) of rigid tubular portion (332) which engages a proximal portion (335) of distal portion (334) as distal portion (334) is rotated away from the longitudinal axis defined by rigid tubular portion (332). This may prevent distal portion (334) from being intentionally or incidentally hyperextended.

### D. EXEMPLARY OUTER SHEATH WITH FLEXIBLE DISTAL PORTION AND RIGID TABS

FIGS. 18A and 18B show yet another exemplary alternative outer sheath (360) having a flexible distal portion (364). Outer sheath (360) may be readily incorporated into instrument (10) along with inner tube (230) discussed above. Outer sheath (360) comprises a rigid tubular portion (362) and a distal portion (364). Distal portion (364) is secured to rigid tubular portion (362) via a flexible portion (366). Flexible portion (366) is defined by a rectangular slot (368) formed within outer sheath (360). Flexible portion (366) is operable to selectively position distal portion (364) at various lateral deflection angles relative to a longitudinal axis defined by rigid tubular portion (362). As should be understood from the discussion above, distal portion (364) is operable to deflect to provide for rotation of clamp arm (44). Distal portion (364) comprises a distally projecting rigid tongue (368). Rigid tongue (368) comprises a pair of flanges (370, 372). Each flange (370, 372) comprises a circular through hole (374, 376) and a tissue stop (378, 380) extending distally from each flange (370, 372) respectively. Clamp arm (44) is pivotably secured to rigid tongue (368) of outer sheath (360) via pin (42) rotatably disposed within through holes (374, 376). Slots (154, 155) of clamp arm (44) are configured to slidably receive tissue stops (378, 380) to thereby permit complete closure of clamp arm (44) into the closed position as discussed above.

Each flange (370, 372) further comprises a proximally extending rigid tab (382, 384). As shown in FIG. 18B, rotation of distal portion (364) is limited by rigid tabs (382, 384) engaging a bottom surface (339) of rectangular slot (368) as distal portion (364) is rotated away from the longitudinal axis defined by rigid tubular portion (362). This may prevent distal portion (364) from being intentionally or incidentally hyperextended.

### III. EXEMPLARY ALTERNATIVE CLAMP ARM OPERATION

It may be desirable to provide an alternative path of rotation to clamp arm (44).

As will be discussed in more detail below, FIGS. 19-24C show various configurations through which a path of rotation of clamp arm (44) may be changed. While various examples by which a path of rotation of clamp arm (44) may be changed will be described in greater detail below, other examples will be apparent to those of ordinary skill in the art in view of the teachings herein. It should be understood that clamp arm (44) of the present example is configured to operate substantially similar to clamp arm (44) discussed above. In particular, clamp arm (44) is operable to compress tissue against blade (160) to thereby sever the tissue and denature the proteins in adjacent tissue cells, thereby providing a coagulative effect with relatively little thermal spread. It should also be understood that, in the examples described below, the inner tubes and outer sheaths may be rigid along their full length, such that neither the inner tube nor the outer sheath needs a flexible or pivoting portion to accommodate closure of clamp arm (44).

### A. EXEMPLARY CLAMP ARM COUPLING WITH DOUBLE DOGLEG PIN

FIGS. 19-21B show an exemplary configuration through which a path of rotation of clamp arm (44) may be changed. In particular, a pin (400) is used to change the path of rotation of clamp arm (44). Pin (400) is configured to operate substantially similar to pin (42) discussed above except for the differences discussed below. In particular, pin (400) pivotably couples clamp arm (44) with outer sheath (132). As best seen in FIG. 19, pin (400) comprises a middle portion (402) and a pair of end portions (404, 406). End portions (404, 406) are connected to and offset from middle portion (402) by a pair of intermediate portions (408, 410) extending substantially perpendicularly between middle portion (402) and end portions (404, 406). As best seen in FIG. 20, middle portion (402) of pin (400) is rotatably disposed within tongue (43) of outer sheath (132), and end portions (404, 406) are rotatably disposed within clamp arm (44) such that end portions (404, 406) orbit about a longitudinal axis defined by middle portion (402) and such that clamp arm (44) is operable to rotate about pin (400) along a path of rotation defined by intermediate portions (408, 410) relative to outer sheath (132). As will be understood from the discussion below, it may be desirable to provide recesses (412, 414) within both sides of tongue (43) to accommodate rotation of intermediate portions (408, 410) between tongue (43) and clamp arm (44).

The present example is discussed as using outer sheath (132) and inner tube (230), both of which are completely rigid and provide no flexing to accommodate for movement of clamp arm (44) toward or away from blade (160). As will be appreciated from the discussion below, pin (400) is configured to accommodate for this lack of flexing within outer sheath (132) and inner tube (230). It should be understood, however, that outer sheath (132) and/or inner tube (230) of the present example may be replaced with any of the examples of outer sheaths and/or inner tubes discussed herein.

FIGS. 21A and 21B show the operation of clamp arm (44) between an open position (FIG. 21A) and a closed position (FIG. 21B). As shown in FIG. 21A, when inner tube (230) is in a distal position relative to outer sheath (132), clamp arm (44) is in the open position. With clamp arm (44) in the open position, pin (400) is oriented obliquely relative to a vertical plane, such that end portions (404, 406) are vertically offset relative to middle portion (402). This angular alignment of middle portion (402) and end portions (404, 406) correlates with middle portion (402) of pin (400) being vertically offset from pins (151, 153) of clamp arm (44). Furthermore, with clamp arm (44) in the open position, the distal ends of tissue stops (136, 137) extend distally of concave surface (158) of clamp arm (44) to thereby inhibit proximal movement of tissue beyond blade (160). As shown in FIG. 15B, as inner tube (230) is moved proximally into a proximal position, clamp arm (44) is pivoted toward blade (160) into the closed position. As clamp arm (44) is moved into the closed position, end portions (404, 406) of pin (400) orbit about the longitudinal axis defined by middle portion (402). Thus, as clamp arm (44) is moved into the closed position, clamp arm (44) rotates about middle portion (402) and end portions (404, 406) of pin (400) along the path of rotation defined by intermediate portions (408, 410) into a position in which pin (400) is oriented substantially vertically such that end portions (404, 406) are aligned vertically relative to middle portion (402). This vertical alignment of middle portion (402) and end portions (404, 406) correlates with middle portion (402) of pin (400) being substantially vertically aligned with pins (151, 153) of clamp arm (44). This vertical alignment would cause deflection of distal portion (234) of inner tube (230) away from the longitudinal axis defined by outer sheath (132) if pin (400) were straight. However intermediate portions (408, 410) provide for added distance between middle portion (402) of pin (400) and pins (151, 153) of clamp arm (44) as clamp arm (44) pivots between the open position and the closed position. Thus it should be appreciated that the path of rotation provided by pin (400) alleviates the need to have outer sheath (132) and/or inner tube (230) be flexible. Furthermore, with clamp arm (44) in the closed position, the distal ends of tissue stops (136, 137) remain aligned substantially adjacent with concave surface (158) of clamp arm (44) to thereby inhibit proximal movement of tissue beyond blade (160).

### B. EXEMPLARY CLAMP ARM COUPLING WITH OFFSET ROTATING LINK

FIGS. 22-24C show another exemplary configuration through which a path of rotation of clamp arm (44) may be manipulated. In particular, a rotatable link (450) is used to manipulate the path of rotation of clamp arm (44). Rotatable link (450) is configured to operate substantially similar to pin (42, 400) discussed above except for the differences discussed below. In particular, rotatable link (450) pivotably couples clamp arm (44) with outer sheath (132). As best seen in FIG. 22, rotatable link (450) comprises an intermediate portion (452) and a pair of cylindrical projections (454, 456) extending laterally from opposite sides of intermediate portion (452). Cylindrical projections (454, 456) are separated by a distance along the length of intermediate portion (452). As best seen in FIG. 23, a pair of rotatable links (450) are positioned between tongue (43) of outer sheath (132) and clamp arm (44). Cylindrical projections (456) of rotatable links (450) are rotatably disposed within tongue (43) of outer sheath (132), and cylindrical projections (454) of rotatable links (450) are rotatably disposed within clamp arm (44) such that clamp arm (44) is operable to rotate along a path of rotation defined by intermediate portion (452) about rotatable link (450) relative to outer sheath (132). As will be understood from the discussion below, it may be desirable to provide recesses (462, 464) within both sides of tongue (43) to accommodate rotation of intermediate portions (452) of rotatable links (450) between tongue (43) and clamp arm (44).

The present example is discussed as using outer sheath (132) and inner tube (230), both of which are completely rigid and provide no flexing to accommodate for movement of clamp arm (44) toward or away from blade (160). As will be appreciated from the discussion below, rotatable link (450) is configured to accommodate for this lack of flexing within outer sheath (132) and inner tube (230). It should be understood, however, that outer sheath (132) and/or inner tube (230) of the present example may be replaced with any of the examples of outer sheaths and/or inner tubes discussed herein.

FIGS. 24A-24C show the operation of clamp arm (44) between an open position (FIG. 24A) and a closed position (FIG. 24C). As shown in FIG. 24A, when inner tube (230) is in a distal position relative to outer sheath (132), clamp arm (44) is in the open position. With clamp arm (44) in the open position, rotatable link (450) is oriented substantially horizontally such that cylindrical projections (454, 456) are aligned substantially horizontally relative to one another.

As shown in FIG. 24B, as inner tube (230) is moved proximally into an intermediate position, clamp arm (44) is pivoted into an intermediate position such that a distal tip of clamp pad (46) of clamp arm (44) contacts blade (160). With clamp arm (44) in the intermediate position, rotatable link (450) is rotated counter-clockwise about a longitudinal axis defined by cylindrical projection (456) and becomes oriented angularly such that cylindrical projections (454, 456) are vertically offset relative to one another.

As shown in FIG. 24C, as inner tube (230) is moved further proximally into a distal position, clamp arm (44) is pivoted into the closed position. Thus, it should be appreciated that rotation of clamp arm (44) from the intermediate position to the closed position has a “squeezing” effect upon tissue compressed between clamp arm (44) and blade (160). With clamp arm (44) in the closed position, rotatable link (450) is rotated clockwise about the longitudinal axis defined by cylindrical projection (456) and becomes oriented angularly such that cylindrical projections (454, 456) are vertically offset relative to one another. Thus it should be appreciated that the path of rotation provided by rotatable link (450) alleviates the need to have outer sheath (132) and/or inner tube (230) be flexible. It should also be understood that rotatable link (450) angularly oscillates about the longitudinal axis defined by cylindrical projection (456) during the closure stroke of clamp arm (44). In particular, projection (454) orbits distally about projection (456) during the transition from the state shown in FIG. 24A to the state shown in FIG. 24B; then projection (454) orbits proximally about projection (456) during the transition from the state shown in FIG. 24B to the state shown in FIG. 24C.

### IV. EXEMPLARY FEATURES TO ADDRESS CLAMP ARM HYPEREXTENSION

During operation, an operator may erroneously attempt to insert end effector (40) into a trocar port while clamp arm (44) is in an open position (as shown in FIG. 12A) when end effector (40) should instead be inserted into the trocar while clamp arm (44) is in a closed position (as shown in FIG. 12C). Such misuse of end effector (40) may result in clamp arm (44) reaching a hyperextended state, where clamp arm (44) is opened further past the proper open position. Thus, it may be desirable to physically prevent clamp arm (44) from reaching a hyperextended state. In the addition or in the alternative, it may be desirable to prevent clamp arm (44) from being closed after clamp arm (44) reaches a hyperextended state, requiring the operator to replace instrument (10) (or at least shaft assembly (30) or end effector (40)) in order to continue with the surgical procedure. As will be discussed in more detail below, FIGS. 25A-36B show various configurations through which hyperextension of clamp arm (44) may be prevented or otherwise dealt with. While several illustrative examples are described in greater detail below, other examples will be apparent to those of ordinary skill in the art in view of the teachings herein. It should be understood that the following examples may be readily incorporated into instrument (10), and may be configured to operate with clamp arm (44) discussed above.

### A. EXEMPLARY ROTATION LIMITING “NACELLE” FLANGES

FIGS. 25A-26 show an exemplary configuration of shaft assembly (30) by which rotation of clamp arm (44) may be limited. In the present example, “nacelle” flanges (180, 181) of distal portion (170) of inner tube (176) are resiliently biased to extend laterally outwardly. During normal operation, as exemplified in FIG. 25A, between the open position of FIG. 12A and 25A and the closed position of FIG. 12B, flanges (180, 181) will remain contained within outer sheath (132). However, if clamp arm (44) is opened too far (i.e., to a hyperextended position), as shown in FIG. 25B, flanges (180, 181) become exposed. This is because rotational movement of clamp arm (44) toward the hyperextended position shown in FIG. 25B causes distal longitudinal movement of inner tube (176) thus exposing flanges (180, 181). When flanges (180, 181) are exposed, and are no longer contained by outer sheath (132), flanges (180, 181) flare outwardly as best shown in FIG. 26. Once flared out, proximal ends of flanges (180, 181) become aligned with a distal edge or face (185) of outer sheath (132) such that inner tube (176) may no longer be moved longitudinally proximally, and such that clamp arm (44) may no longer be closed. This may require the operator to dispose of instrument (10) and retrieve a new instrument (10) in order to perform a surgical procedure. Additionally or alternatively, in those versions of instrument (10) where shaft assembly (30) and end effector (40) are selectively removable from instrument (10), the operator may be required to remove shaft assembly (30) and end effector (40) from instrument (10) and dispose of shaft assembly (30) and end effector (40) and retrieve and attach a new shaft assembly (30) and end effector (40) in order to perform a surgical procedure.

Although flanges (180, 181) of the present example are discussed as being outwardly biased so as to align with distal face (185) of outer sheath (132), it should be appreciated that flanges (180, 181) may be laterally outwardly biased so as to extend beyond an exterior surface of outer sheath (132) such that clamp arm (44) may no longer be completely closed.

### B. EXEMPLARY ROTATION LIMITING TISSUE STOPS

FIGS. 27A-28 show another exemplary configuration of shaft assembly (30) by which rotation of clamp arm (44) may be limited. In the present example, tissue stops (136, 137) of distal portion (170) of outer sheath (132) are resiliently biased laterally inwardly. During normal operation, as exemplified in FIG. 27A, between the open position of FIG. 12A and the closed position of FIG. 12B, tissue stops (136, 137) will remain substantially straight because of contact with interior surfaces of slots (154, 155) and/or secondary clamp pad (48). However, if clamp arm (44) is opened too far (i.e., to a hyperextended position), as shown in FIG. 27B, tissue stops (136, 137) are no longer in contact with either the interior surfaces of slots (154, 155) or secondary clamp pad (48). When tissue stops (136, 137) are no longer in contact with either the interior surfaces of slots (154, 155) or secondary clamp pad (48), tissue stops (136, 137) bend inwardly as best shown in FIG. 28. Once bent inwardly, top surfaces of tissue stops (136, 137) are positioned below secondary clamp pad (48), out of alignment with slots (154, 155), such that clamp arm (44) may no longer be closed. This may require the operator to dispose of instrument (10) and retrieve a new instrument (10) in order to perform a surgical procedure. Additionally or alternatively, in those versions of instrument (10) where shaft assembly (30) and end effector (40) are selectively removable from instrument (10), the operator may be required to remove shaft assembly (30) and end effector (40) from instrument (10) and dispose of shaft assembly (30) and end effector (40) and retrieve and attach a new shaft assembly (30) and end effector (40) in order to perform a surgical procedure.

### C. Exemplary Rotation Limiting Outer Sheath and Inner Tube

FIGS. 29A and 29B show yet another exemplary configuration of shaft assembly (30) by which rotation of clamp arm (44) may be limited. In the present example, inner tube (176) comprises an outwardly biased tab (177) and outer sheath (132) comprises a lateral opening (187). Lateral opening (187) of outer sheath (132) is sized to receive tab (177) of inner tube (176). As discussed above, inner tube (176) is operable to translate longitudinally within outer sheath (132) relative to outer sheath (132) to selectively pivot clamp arm (44) toward and away from blade (160). During normal operation, as exemplified in FIG. 29A, between the open position of FIG. 12A and the closed position of FIG. 12B, tab (177) will remain contained within outer sheath (132), bearing against an interior surface of outer sheath (132). However, if clamp arm (44) is opened too far (i.e., to a hyperextended position), thus driving inner tube (176) too far distally as shown in FIG. 25B, tab (177) will become aligned with lateral opening (187) and flare outwardly within lateral opening (187). Once flared out, a proximal surface (179) of tab (177) becomes aligned with a distal face (189) of outer sheath (132) such that inner tube (176) may no longer be moved longitudinally proximally and such that clamp arm (44) may no longer be closed. This may require the operator to dispose of instrument (10) and retrieve a new instrument (10) in order to perform a surgical procedure. Additionally or alternatively, in those versions of instrument (10) where shaft assembly (30) and end effector (40) are selectively removable from instrument (10), the operator may be required to remove shaft assembly (30) and end effector (40) from instrument (10) and dispose of shaft assembly (30) and end effector (40) and retrieve and attach a new shaft assembly (30) and end effector (40) in order to perform a surgical procedure.

### D. EXEMPLARY ROTATION LIMITING INNER TUBE

FIGS. 30-31B show an exemplary configuration of inner tube (176) by which rotation of clamp arm (44) may be limited. In the present example, inner tube (176) comprises an arcuate member (190) connecting proximal portions of “nacelle” flanges (180, 181) to one another. During normal operation of clamp arm (44) between the open position of FIG. 12A and the closed position of FIG. 12B, a proximal surface (191) of arcuate member (190) will not contact a distal edge or surface (192) of rigid tubular portion (178) of inner tube (176). However, if clamp arm (44) is opened slightly past the normal open position of FIG. 12A (but not necessarily yet reaching a hyperextended position), distal portion (170) will bend upwardly such that proximal surface (191) of arcuate member (190) engages distal surface (192) of rigid tubular portion (178), as shown in FIG. 31B, thereby limiting the ability of clamp arm (44) to open any further. In some such versions, clamp arm (44) may subsequently return to the normal open position (as shown in FIG. 12A) and be driven to the closed position (as shown in FIG. 12B). Thus, the operator may continue using the instrument (10) incorporating a version of inner tube (176) with arcuate member (190).

### E. EXEMPLARY ROTATION LIMITING INNER TUBE WITH ENGAGEMENT POSTS

FIGS. 32-33B show another exemplary configuration of inner tube (176) by which rotation of clamp arm (44) may be limited. In the present example, inner tube (176) comprises a pair of posts (193, 194) extending upwardly and integrally from an interior surface of base (171) of distal portion (170). Posts (193, 194) are configured and positioned such that they are located on opposite lateral sides of waveguide (184); and such that posts (193, 194) do not contact waveguide (184). During normal operation of clamp arm (44) between the open position of FIG. 12A and the closed position of FIG. 12B, a top surface (195, 196) of each post (193, 194) will not contact an interior surface of outer sheath (132). However, if clamp arm (44) is opened slightly past the normal open position of FIG. 12A (but not necessarily yet reaching a hyperextended position), distal portion (170) will bend upwardly such that top surfaces (195, 196) of posts (193, 194) will engage the interior surface of outer sheath (132), as shown in FIG. 33B, thereby limiting the ability of clamp arm (44) to open any further. In some such versions, clamp arm (44) may subsequently return to the normal open position (as shown in FIG. 12A) and be driven to the closed position (as shown in FIG. 12B). Thus, the operator may continue using the instrument (10) incorporating a version of inner tube (176) with posts (193, 194).

### F. EXEMPLARY ROTATION LIMITING INNER TUBE WITH ENGAGEMENT PAD

FIGS. 34-35B show yet another exemplary configuration of inner tube (176) by which rotation of clamp arm (44) may be limited. In the present example, inner tube (176) comprises a rectangular pad (197) extending upwardly from the interior surface of base (171) of distal portion (170). Rectangular pad (197) may comprise polytetrafluoroethylene (“PTFE”) or any other appropriate material. During normal operation of clamp arm (44) between the open position of FIG. 12A and the closed position of FIG. 12B, a top surface (198) of rectangular pad (197) will not contact a bottom surface of blade (160) or waveguide (184). However, if clamp arm (44) is opened slightly past the normal open position of FIG. 12A (but not necessarily yet reaching a hyperextended position), distal portion (170) will bend upwardly such that top surface (198) of rectangular pad (197) will engage the bottom surface of blade (160) or waveguide (184), as shown in FIG. 35B, thereby limiting the ability of clamp arm (44) to open any further. In some such versions, clamp arm (44) may subsequently return to the normal open position (as shown in FIG. 12A) and be driven to the closed position (as shown in FIG. 12B). Thus, the operator may continue using the instrument (10) incorporating a version of inner tube (176) with rectangular pad (197). It should be understood that pad (197) may be of any suitable shape, and need not necessarily be rectangular.

### G. EXEMPLARY ROTATION LIMITING CLAMP ARM AND OUTER SHEATH

FIGS. 36A and 36B show yet another exemplary configuration of clamp arm (44) and outer sheath (132) by which rotation of clamp arm (44) may be limited. In the present example, tongue (43) comprises a distally extending tab (199). Clamp arm (44) of the present example comprises a recess (47) formed in a proximal end of clamp arm (44). Tab (199) is rotatably disposed within recess (47) of clamp arm (44). During normal operation of clamp arm (44) between the open position of FIG. 12A and the closed position of FIG. 12B, tab (199) will not contact a bottom surface (49) of recess (47). However, if clamp arm (44) is opened slightly past the normal open position of FIG. 12A (but not necessarily yet reaching a hyperextended position), as shown in FIG. 36B, tab (199) will engage bottom surface (49) of recess (47) thereby limiting the ability of clamp arm (44) to open any further.

### V. EXEMPLARY SHAFT ASSEMBLY AND/OR END EFFECTOR FEATURES

It may be desirable to provide shaft assembly (30) and/or end effector (40) with features to improve the ease of use and/or the effectiveness of instrument (10). As will be discussed in more detail below, FIGS. 37-45 show various examples of shaft assemblies and end effectors operable to improve the ease of use and/or the effectiveness of instrument (10). While various examples of how to improve the ease of use and/or the effectiveness of instrument (10) will be described in greater detail below, other examples will be apparent to those of ordinary skill in the art in view of the teachings herein. It should be understood that the following examples may be readily incorporated into instrument (10) discussed above.

### A. EXEMPLARY TWO-PIECE INNER TUBE

FIGS. 37-40 show an exemplary alternative inner tube (500). Inner tube (500) may be readily incorporated into instrument (10) discussed above. Inner tube (500) is configured to operate substantially similar to inner tube (176) discussed above except for the differences discussed below. In particular, inner tube (500) is operable to translate longitudinally within outer sheath (132) relative to outer sheath (132) to selectively pivot clamp arm (44) toward and away from blade (160). Inner tube (500) comprises a rigid tubular portion (502) and a resilient distal portion (504). Distal portion (504) is coupled to a distal end of rigid tubular portion (502) via a semi-circular connector (505). For instance, connector (505) of distal portion (504) may be coupled to the distal end of rigid tubular portion (502) in a snap-fit configuration with a semi-circular recess (507) of rigid tubular portion (502). As best seen in FIG. 40, distal portion (504) comprises a flexible portion (506). Flexible portion (506) is operable to selectively position distal portion (504) at various lateral deflection angles relative to a longitudinal axis defined by rigid tubular portion (502). Flexible portion (506) is defined by a pair of slots (517, 519) formed within distal portion (504). Slots (517, 519) provide flexibility to flexible portion (506) and further define a pair of “nacelle” flanges (518, 520) as will be discussed in more detail below. As will be discussed on more detail below, distal portion (504) is operable to flex to provide for rotation of clamp arm (44). Distal portion (504) may comprise a plastic, or any other resilient and/or flexible material.

Distal portion (504) comprises a pair of flanges (508, 510) extending from a base (512). Each flange (508, 510) comprises a circular through hole (514, 516) and a “nacelle” flange (518, 520) extending proximally from each flange (508, 510) respectively. Clamp arm (44) is pivotably secured to flanges (508, 510) of distal portion (504) via inwardly extending pins (151, 153) of arms (156) rotatably disposed within through holes (514, 516). Inner tube (500) is operable to translate longitudinally within outer sheath (132) relative to outer sheath (132) to selectively pivot clamp arm (44) toward and away from blade (160). In particular, inner tube (500) is coupled with trigger (28) such that clamp arm (44) pivots toward blade (160) in response to pivoting of trigger (28) toward pistol grip (24); and such that clamp arm (44) pivots away from blade (160) in response to pivoting of trigger (28) away from pistol grip (24). Clamp arm (44) may be biased toward the open position, such that (at least in some instances) the operator may effectively open clamp arm (44) by releasing a grip on trigger (28).

### B. EXEMPLARY SHAFT ASSEMBLY WITH FEEDBACK FEATURES

FIGS. 41-42B show an exemplary alternative inner tube (550) and outer sheath (580) that may be readily incorporated into instrument (10) discussed above. Inner tube (550) is configured to operate substantially similar to inner tube (176) discussed above except for the differences discussed below. In particular, inner tube (550) is operable to translate longitudinally within outer sheath (580) relative to outer sheath (580) to selectively pivot clamp arm (44) toward and away from blade (160). As with inner tube (176) discussed above, inner tube (550) comprises a pair of “nacelle” flanges (552, 554). Flange (552) comprises a spherical projection (556). Outer sheath (580) comprises a pair of detents (582, 584) formed in outer sheath (580), extending outwardly from an interior surface in outer sheath (580). Spherical projection (556) of inner tube (550) is configured to successively engage detents (582, 584) of outer sheath (580) as inner tube (550) translates longitudinally within outer sheath (580). For instance, as shown in FIGS. 42A and 42B, as clamp arm (44) moves from an open position (FIG. 42A) toward a closed position (FIG. 42B), spherical projection (556) of inner tube (550) engages detents (582, 584) of outer sheath (580) as inner tube (550) translates longitudinally within outer sheath (580). It should be appreciated that engagement of spherical projection (556) of inner tube (550) and detents (582, 584) of outer sheath (580) may provide audible and/or tactile feedback to an operator of instrument (10). In particular, teach time projection (556) pops into a detent (582, 584), an audible and/or tactile click/pop may be emitted through shaft assembly (30).

Although spherical projection (556) of inner tube (550) is described as only being disposed on flange (552), it should be understood that spherical projections may be formed on flange (554) and corresponding detents may be formed in outer sheath (580). It should also be understood that detents (582, 584) may be positioned along outer sheath (580) at locations corresponding with particular rotational positions of clamp arm (44). For instance, detent (582) may correspond with an intermediate rotational position (i.e., a partially closed position) and/or detent (584) may correspond with the fully closed position such that the user may be made aware through audible and/or tactile feedback that clamp arm (44) is partially or completely closed. It should also be appreciated that detents (582, 584) may be positioned along an arcuate path to accommodate deflection of inner tube (500) during closure of clamp arm (44), as described above with respect to inner tube (176) with reference to FIGS. 12A-12C.

### C. EXEMPLARY SHAFT ASSEMBLY WITH CLEANING FEATURES

FIGS. 43-45 show an exemplary alternative inner tube (600). Inner tube (600) may be readily incorporated into instrument (10) discussed above. Inner tube (600) is configured to operate substantially similar to inner tube (176) discussed above except for the differences discussed below. In particular, inner tube (600) is operable to translate longitudinally within outer sheath (132) relative to outer sheath (132) to selectively pivot clamp arm (44) toward and away from blade (160). Inner tube (600) of the present example comprises a rectangular opening (602) formed in a flexible portion (604) of inner tube (600). Opening (602) is configured to provide access to an interior of shaft assembly (30) and/or end effector (40) such that the interior of shaft assembly (30) and/or end effector (40) may be cleaned, e.g. flushed, vacuumed, brushed, scraped, etc. via opening (602). For instance, tissue, coagulated blood, and/or fluid, etc. may be cleaned from the interior of shaft assembly (30) and/or end effector (40) via opening (552).

Although opening (602) is described as being formed in flexible portion (604) of inner tube (600), it should be understood that opening (602) may be formed at any appropriate position along inner tube (600) and/or outer sheath (132). Also, although opening (602) is described as being rectangular, it should be understood that opening (602) may have any other suitable shape.

### VI. EXEMPLARY ALTERNATIVE CLAMP ARM AND SHAFT ASSEMBLY OPERATION

It may be desirable to provide shaft assembly (30) and/or end effector (40) with features operable to change the method of operation and/or actuation of end effector (40). As will be discussed in more detail below, FIGS. 46A-61 show various examples of features that may be incorporated into shaft assemblies and end effectors to change the method of operation and/or actuation of end effector (40). While various examples of how to change the method of operation and/or actuation of end effector (40) will be described in greater detail below, other examples will be apparent to those of ordinary skill in the art in view of the teachings herein. It should be understood that the following examples may be readily incorporated into instrument (10) discussed above.

### A. EXEMPLARY INNER TUBE WITH ROTATABLE LINK MEMBER

FIGS. 46A-46C show an exemplary alternative inner tube (700). Inner tube (700) may be readily incorporated into instrument (10) discussed above. Inner tube (700) is configured to operate substantially similar to inner tube (176) discussed above except for the differences discussed below. In particular, inner tube (700) is operable to translate longitudinally within outer sheath (132) relative to outer sheath (132) to selectively pivot clamp arm (44) toward and away from blade (160). Outer sheath (132) and inner tube (700) of the present example are both completely rigid along their respective lengths and provide no flexing to accommodate for movement of clamp arm (44) toward or away from blade (160). Instead, a link member (702) is provided to accommodate for this lack of flexing in outer sheath (132) and inner tube (700). A proximal end of link member (702) is rotatably coupled to a distal end of inner tube (700). A distal end of link member (702) is rotatably coupled with arm (156) of clamp arm (44). Thus, it should be appreciated that longitudinal translation of inner tube (700) will be communicated to clamp arm (44) via link member (702) to thereby cause rotation of clamp arm (44) about pin (42).

FIGS. 46A-46C show operation of clamp arm (44) between an open position (FIG. 46A) and a closed position (FIG. 46C). As shown in FIG. 46A, when inner tube (700) is in a distal position relative to outer sheath (132), clamp arm (44) is in the open position. With clamp arm (44) in the open position, link member (702) is in a first oblique orientation relative to a longitudinal axis (C) defined by inner tube (700). As shown in FIG. 46B, as inner tube (700) is moved proximally into an intermediate position, clamp arm (44) is pivoted toward blade (160) to an intermediate position. As clamp arm (44) is moved to the intermediate position, link member (702) is moved to a second oblique orientation relative to longitudinal axis (C). As shown in FIG. 46C, as inner tube (700) is moved further proximally into a proximal position, clamp arm (44) is pivoted toward blade (160) to the closed position. As clamp arm (44) is moved to the closed position, link member (702) is moved to a third oblique orientation relative to longitudinal axis (C). It should therefore be understood that the change in position of link member (702) between the first oblique orientation and the third oblique orientation will accommodate for the lack of flexing within outer sheath (132) and inner tube (700) to thereby to accommodate for movement of clamp arm (44) toward or away from blade (160). It should also be understood that link member (702) pivots in the same angular direction as clamp arm (44) while clamp arm (44) pivots from the open position (FIG. 46A) to the closed position (FIG. 46C).

Although the present example is discussed as having only a single link member (702), it should be appreciated that any appropriate number of link members (702) may be used. For instance, a pair of link members (702) may be positioned on opposite sides of blade (160) to thereby connect each arm (156) of clamp arm (44) with inner tube (700). As another merely illustrative alternative, link (702) may be broken into two or more links that are pivotally coupled together to join inner tube (700) with arm (156) of clamp arm (44).

### B. EXEMPLARY CLAMP ARM WITH ELONGATE SLOTS

FIGS. 47-51B show an exemplary alternative inner tube (710) and clamp arm (730). Inner tube (710) and clamp arm (730) may be readily incorporated into instrument (10) discussed above. Inner tube (710) is configured to operate substantially similar to inner tube (176) discussed above except for the differences discussed below. In particular, inner tube (710) is operable to translate longitudinally within outer sheath (132) relative to outer sheath (132) to selectively pivot clamp arm (730) toward and away from blade (160). Clamp arm (730) of the present example is configured to operate substantially similar to clamp arm (44) discussed above. In particular, clamp arm (730) is operable to compress tissue against blade (160) to thereby sever the tissue and denature the proteins in adjacent tissue cells, thereby providing a coagulative effect with relatively little thermal spread. Outer sheath (132) and inner tube (730) of the present example are completely rigid along their respective lengths and provide no flexing to accommodate for movement of clamp arm (730) toward or away from blade (160). As will be appreciated from the discussion below, however, clamp arm (730) is configured to accommodate for this lack of flexing within outer sheath (132) and inner tube (710).

Clamp arm (730) includes a primary clamp pad (738) and a secondary clamp pad (1740) that are secured to the underside of clamp arm (730), facing blade (160). Clamp arm (730) is pivotably secured to tongue (43) of outer sheath (132) via pin (42). Pin (42) has a circular cross-sectional profile. A pair of arms (732) extend transversely from clamp arm (730) and are secured to a distal end of inner tube (710) that extends laterally about arms (732). Inner tube (710) comprises a pair of integral, inwardly extending pins (712, 714). Pins (712, 714) each have a circular cross-sectional profile. Arms (732) are rotatably secured to the distal end of inner tube (710) via pins (712, 714), which are rotatably disposed within a pair of elongate slots (734, 736) formed in arms (732). As best seen in FIG. 48, slots (734, 736) are oblong, such that slots (734, 736) are non-circular. Pins (712, 714) are configured to translate within elongate slots (734, 736) between a first position and a second position. Thus, it should be appreciated that longitudinal translation of inner tube (710) will be communicated to clamp arm (730) via pins (712, 714) disposed within elongate slots (734, 736) to thereby cause rotation of clamp arm (730) about pin (42).

The elongate configuration of slots (734, 736) will provide clearance for pins (712, 714) to travel along slots (734, 736) to accommodate movement of arms (732) during closure of clamp arm (730). FIGS. 51A and 51B show operation of clamp arm (730) between an open position (FIG. 51A) and a closed position (FIG. 51B). As shown in FIG. 51A, when inner tube (710) is in a distal position relative to outer sheath (132), clamp arm (730) is in the open position. With clamp arm (730) in the open position, pins (712, 714) of inner tube (710) are disposed within elongate slots (734, 736) of arms (732) in a first position. As shown in FIG. 51B, as inner tube (700) is moved proximally into a proximal position, clamp arm (730) is pivoted toward blade (160) into the closed position. As clamp arm (730) is moved into the closed position, pins (712, 714) of inner tube (710) are translated within elongate slots (734, 736) into a second position. It should therefore be understood that the translation of pins (712, 714) within elongate slots (734, 736) between the first position and the second position will accommodate for the lack of flexing within outer sheath (132) and inner tube (710) to thereby to accommodate for movement of clamp arm (730) toward or away from blade (160).

### C. EXEMPLARY OUTER SHEATH WITH ELONGATE SLOTS

FIGS. 52-53B show an exemplary alternative outer sheath (750). Outer sheath (750) may be readily incorporated into instrument (10) discussed above. Outer sheath (750) is configured to operate substantially similar to outer sheath (132) discussed above except for the differences discussed below. Inner tube (230), discussed above, and outer sheath (750) of the present example are completely rigid along their respective lengths and provide no flexing to accommodate for movement of clamp arm (44) toward or away from blade (160). As will be appreciated from the discussion below, however, outer sheath (750) is configured to accommodate for this lack of flexing within outer sheath (750) and inner tube (230).

Outer sheath (750) comprises a distally projecting tongue (752). Tongue (752) comprises a pair of flanges (751, 753). Each flanged comprises an elongate slot (754, 756) formed therein. As best seen in FIG. 52, slots (754, 756) are oblong, such that slots (754, 756) are non-circular. Clamp arm (44) is pivotably secured to tongue (752) of outer sheath (750) via pin (42), which is rotatably disposed within elongate slots (754, 756). Thus, clamp arm (44) is operable to selectively pivot about pin (42) within elongate slots (754, 756) toward and away from blade (160) to selectively clamp tissue between clamp arm (44) and blade (160). Pin (42) has a circular cross-sectional profile. Pin (42) is configured to translate within elongate slots (754, 756) between a first position and a second position such that clamp arm (44) is also able to translate within elongate slots (754, 756). Tongue (752) further comprises a pair of tissue stops (758, 760) configured to operate substantially similar to tissue stops (136, 137) discussed above. In particular, tissue stops (758, 760) are configured to inhibit proximal movement of tissue beyond blade (160) and/or into the interior of outer sheath (750) and/or inner tube (230).

Clamp arm (44) is pivotably secured to inner tube (230) through a combination of pins and openings, with the pins having circular cross-sectional profiles and the openings having circular shapes. FIGS. 53A and 53B show operation of clamp arm (44) between an open position (FIG. 53A) and a closed position (FIG. 53B). As shown in FIG. 53A, when inner tube (710) is in a distal position relative to outer sheath (132), clamp arm (730) is in the open position. With clamp arm (730) in the open position, pins (712, 714) of inner tube (710) are disposed within elongate slots (734, 736) of arms (732) in a first position. As shown in FIG. 53B, as inner tube (700) is moved proximally into a proximal position, clamp arm (730) is pivoted toward blade (160) into the closed position. As clamp arm (730) is moved into the closed position, pins (712, 714) of inner tube (710) are translated within elongate slots (734, 736) into a second position. It should therefore be understood that the translation of pins (712, 714) within elongate slots (734, 736) between the first position and the second position will accommodate for the lack of flexing within outer sheath (132) and inner tube (710) to thereby to accommodate for movement of clamp arm (730) toward or away from blade (160).

### D. Exemplary Outer Sheath with Integral Drive Features and Tissue Stop

FIGS. 54-56 show an exemplary alternative shaft assembly (800) and end effector (840). Shaft assembly (800) and end effector (840) may be readily incorporated into instrument (10) discussed above. End effector (840) of the present example comprises clamp arm (844) and ultrasonic blade (160). Clamp arm (844) is pivotably secured to a distal end of a collar (802) of shaft assembly (800). Clamp arm (844) is operable to selectively pivot toward and away from blade (160) to selectively clamp tissue between clamp arm (844) and blade (160). A pair of arms (846) extend transversely from clamp arm (844) and are rotatably secured to collar (802). Each arm (846) comprises an integral, outwardly extending pin (848, 850). Arms (846) are rotatably secured to collar (802) via pins (848, 850), which are rotatably disposed within a pair of circular through holes (804, 806) of collar (802).

Clamp arm (844) further comprises a tab (852) extending proximally from a proximal surface (845) of clamp arm (844). Tab (852) comprises a through hole (854). Shaft assembly (800) comprises a rod (810) and an outer sheath (808). Collar (802) is fixedly secured to a distal end of outer sheath (808). Rod (810) is slidably disposed within a longitudinal channel (812) formed in a top surface of outer sheath (808). Rod (810) is further slidably disposed within a through hole (814) and a longitudinal channel (816) formed in a top surface of collar (802) such that a distal end of rod (810) may be rotatably secured within through hole (854) of tab (852). As discussed above, clamp arm (844) is pivotably secured to collar (802) via pins (848, 850) of arms (846). Rod (810) is operable to translate longitudinally within channel (812) of outer sheath (808) and within through hole (814) and channel (816) of collar (802) to selectively pivot clamp arm (844) toward and away from blade (160). In particular, rod (810) is operable to translate between a distal position (FIG. 51) and a proximal position (FIG. 52) to thereby pivot clamp arm (844) between a closed position (FIG. 51) and an open position (FIG. 52). Rod (810) may be coupled with trigger (28), discussed above, such that clamp arm (844) pivots toward blade (160) in response to pivoting of trigger (28) toward pistol grip (24); and such that clamp arm (844) pivots away from blade (160) in response to pivoting of trigger (28) away from pistol grip (24). Clamp arm (844) may be biased toward the open position, such that (at least in some instances) the operator may effectively open clamp arm (844) by releasing a grip on trigger (28). In some versions, rod (810) pivots or flexes as clamp arm (844) transitions between an open and closed configuration. Such pivoting or flexing of rod (810) may accommodate displacement of tab (852) toward and away from the longitudinal axis of shaft assembly (800) during opening/closing of clamp arm (844).

Collar (802) of the present example comprises a through bore (803) into which blade (160) and waveguide (184) are disposed, and from which blade (160) distally extends. Through bore (803) is sized such that an interior surface of through bore (803) is sufficiently adjacent to an exterior surface of blade (160) and/or waveguide (184) so as to inhibit proximal movement of tissue beyond blade (160) and/or into the interior of collar (802) and/or outer sheath (808). Thus, it should be understood that a distal surface (805) of collar (802) may be configured to act as a tissue stop such as to inhibit proximal movement of tissue beyond distal surface (805) into the interior of collar (802) and/or outer sheath (808).

### E. EXEMPLARY TISSUE STOP INSERT

FIGS. 57-59B show an exemplary tissue stop insert (850). As will be discussed in more detail below, tissue stop insert (850) is configured to inhibit proximal movement of tissue beyond blade (160) and/or into the interior of outer sheath (132) and/or inner tube (176). Tissue stop insert (850) comprises a through bore (852) through which blade (160) and/or waveguide (184) are disposed, and from which blade (160) distally extends. Through bore (852) is sized such that an interior surface of through bore (852) is sufficiently adjacent to the exterior surface of blade (160) and/or waveguide (184) so as to inhibit proximal movement of tissue. In some versions, a very slight gap is provided between the inner surface of through bore (852) and the outer surface of blade (160) and/or waveguide (184). Such a gap may be large enough to prevent contact between the inner surface of through bore (852) and the outer surface of blade (160) and/or waveguide (184); yet be small enough to prevent tissue from passing into the gap.

A proximal end of tissue stop insert (850) is configured for insertion into the distal end of inner tube (176) and/or outer sheath (132) such that a distal surface (854) of tissue stop insert (850) is substantially aligned with the distal ends of inner tube (176) and outer sheath (132) as best seen in FIG. 58. Distal surface (854) is shaped to substantially follow with the contours of the distal ends of inner tube (176) and outer sheath (132). In particular, distal surface (854) comprises a top portion (856), which substantially aligns with the distal end of tongue (43) of outer sheath (132); and a bottom portion (858), which substantially aligns with a distal end of distal portion (170) of inner tube (176). Top portion (856) further comprises a pair of recesses (860, 862) configured to receive flanges (133, 135). Top portion (856) of distal surface (854) is configured to be positioned distally of secondary clamp pad (48) when inserted into outer sheath (132) and/or inner tube (176). Tissue stop insert (850) comprises a through bore (864) configured to align with through holes (138, 139) of tongue (43) such that pin (42) may be inserted there through to thereby secure tissue stop insert (850) in place. Tissue stop insert (850) may be machined or molded, among other manufacturing methods, and may comprise silicone, rubber, fluoropolymer, or any other appropriate material.

### F. EXEMPLARY TISSUE STOP TUBE

FIGS. 60 and 61 show an exemplary tissue stop tube (900). As will be discussed in more detail below, tissue stop tube (900) is configured to inhibit proximal movement of tissue beyond blade (160) and/or into the interior of outer sheath (132) and/or inner tube (176). Tissue stop tube (900) comprises a through bore (902) through which blade (160) and/or waveguide (184) are disposed, and from which blade (160) distally extends. Through bore (902) is sized such that an interior surface of through bore (902) is sufficiently adjacent to the exterior surface of blade (160) and/or waveguide (184) so as to inhibit proximal movement of tissue. In some versions, a very slight gap is provided between the inner surface of through bore (902) and the outer surface of blade (160) and/or waveguide (184). Such a gap may be large enough to prevent contact between the inner surface of through bore (902) and the outer surface of blade (160) and/or waveguide (184); yet be small enough to prevent tissue from passing into the gap.

A proximal end of tissue stop tube (900) is configured for insertion into the distal end of inner tube (176) and/or outer sheath (132) such that a distal surface (904) of tissue stop tube (900) is substantially aligned with concave surface (158) of arms (156) of clamp arm (44). It should be understood, however, that distal surface (904) may be positioned at any appropriate position relative to concave surface (158). Tissue stop tube (900) may be held in place by engagement with an interior surface of inner tube (176) and an exterior surface of blade (160) and/or waveguide (184). In versions where tissue stop tube (900) engages blade (160) and/or waveguide (184), tissue stop tube (900) may engage blade (160) and/or waveguide (184) at a longitudinal position corresponding to a node associate with ultrasonic vibrations communicated along blade (160) and/or waveguide (184). Tissue stop tube (900) may be extruded, machined, or molded, among other manufacturing methods, and may comprise silicone, rubber, fluoropolymer, or any other appropriate material. It should be understood that the distal end of tissue stop tube (900) may be machined to comprise a semi-circular projection that may be oriented at any rotational position about blade (160) and/or waveguide (184).

### VII. Miscellaneous

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Moreover, those of ordinary skill in the art will recognize that various teachings herein may be readily applied to electrosurgical instruments, stapling instruments, and other kinds of surgical instruments. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometric s, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus for operating on tissue, the apparatus comprising:
(a) a body;
(b) a shaft assembly extending distally from the body, wherein the shaft assembly defines a longitudinal axis, wherein the shaft assembly comprises a translatable member, wherein the translatable member comprises a proximal portion and a distal portion;
(c) an acoustic waveguide, wherein the waveguide is configured to transmit ultrasonic vibrations; and
(d) an end effector comprising:
(i) an ultrasonic blade in acoustic communication with the waveguide, and
(ii) a clamp arm, wherein the clamp arm is operable to pivot toward and away from the blade, wherein the translatable member is configured to translate to thereby cause pivoting of the clamp arm toward and away from the blade;
wherein the distal portion of the translatable member is configured to flex to thereby accommodate pivoting of the clamp arm toward and away from the blade.

2. The apparatus of claim 1, wherein the clamp arm comprises a distally facing feature configured to provide for proximal positioning of tissue between the clamp arm and the blade.

3. The apparatus of claim 2, wherein the distally facing feature of the clamp arm comprises a concave surface.

4. The apparatus of claim 1, wherein the shaft assembly further comprises a stationary member, wherein the stationary member includes at least one tissue stop configured to restrict proximal movement of tissue.

5. The apparatus of claim 4, wherein the clamp arm comprises at least one slot configured to slidably receive the at least one tissue stop.

6. The apparatus of claim 4, wherein the at least one tissue stop is resiliently biased toward a locked position.

7. The apparatus of claim 6, the at least one tissue stop is configured to prevent pivoting of the clamp arm when the at least one tissue stop is in the locked position.

8. The apparatus of claim 1, wherein the shaft assembly further comprises at least one locking member resiliently biased toward a locked position.

9. The apparatus of claim 8, wherein the at least one locking member is configured to prevent pivoting of the clamp arm when the at least one locking member is in the locked position.

10. The apparatus of claim 1, wherein the shaft assembly further comprises a stationary member, wherein the translatable member is configured to longitudinally translate relative to the stationary member between a distal position and a proximal position.

11. The apparatus of claim 10, wherein the stationary member comprises at least one feature configured to provide for flexing of the distal portion of the translatable member.

12. The apparatus of claim 11, wherein the at least one feature comprises a slot formed in the translatable member.

13. The apparatus of claim 1, wherein the translatable member comprises at least one feature configured to provide audible and/or tactile feedback to a user based on a position of the clamp arm relative to the blade.

14. The apparatus of claim 1, wherein he shaft assembly comprises a rotation limiting feature configured to restrict opening movement of the clamp arm relative to the blade.

15. The apparatus of claim 1, wherein the translatable member further comprises a flexible portion, wherein the flexible portion is disposed between the distal portion of the translatable member and the proximal portion of the translatable member.

16. The apparatus of claim 1, wherein the shaft assembly comprises at least one transverse opening configured to provide for cleaning of an interior of the shaft assembly.

17. The apparatus of claim 1, wherein the clamp arm is rotatably secured to the translatable member via a first pin disposed in a first opening, wherein the first pin has a circular cross-sectional profile, wherein the first opening has a circular shape.

18. The apparatus of claim 17, wherein the clamp arm is further rotatably secured to a stationary member of the shaft assembly via a second pin disposed in a second opening, wherein the second pin has a circular cross-sectional profile, wherein the second opening has a circular shape.

19. An apparatus for operating on tissue, the apparatus comprising:
(a) a body;
(b) a shaft assembly extending distally from the body, wherein the shaft assembly defines a longitudinal axis, wherein the shaft assembly comprises:
(i) a translatable member, and
(ii) a stationary member;
(c) an acoustic waveguide, wherein the waveguide is configured to transmit ultrasonic vibrations; and
(d) an end effector comprising:
(i) an ultrasonic blade in acoustic communication with the waveguide,
(ii) a clamp arm, and
(iii) a rotatable member, wherein the clamp arm is rotatably coupled with the stationary member via the rotatable member,
wherein the coupling between the rotatable member and the stationary member is configured to provide for rotation of the rotatable member about a first axis,
wherein the coupling between the rotatable member and the clamp arm is configured to provide for rotation of the clamp arm about a second axis,
wherein the first axis and the second axis are separated by a distance such that the clamp arm is rotatable along a path of rotation defined by the distance and relative to the stationary member to thereby provide for rotation of the clamp arm toward and away from the blade.

20. An apparatus for operating on tissue, the apparatus comprising:
(a) a body;
(b) a shaft assembly extending distally from the body, wherein the shaft assembly defines a longitudinal axis, wherein the shaft assembly comprises a stationary member, wherein the stationary member comprises at least one tissue stop associated with a distal end of the stationary member, wherein the at least one tissue stop is configured to restrict proximal movement of tissue;
(c) an acoustic waveguide, wherein the waveguide is configured to transmit ultrasonic vibrations; and
(d) an end effector comprising:
(i) an ultrasonic blade in acoustic communication with the waveguide, and
(ii) a clamp arm operable to pivot toward and away from the blade, wherein the clamp arm is configured to receive the at least ons tissue stop as the clamp arm pivots toward the blade.
